(19) United States
(12) Patent Application Publication
(10) Pub. No.: US 2002/0028799 A1
(43) Pub. Date: Mar. 7, 2002

(54) Treatment of male sexual dysfunction

(76) Inventors: Alasdair Mark Naylor, Sandwich (GB); Pieter Hadewijn Van Der Graaf, Sandwich (GB); Christopher peter Wayman, Sandwich (GB)

Correspondence Address: Gregg C. Benson, Pfizer Inc., Patent Department, MS 4159, Eastern Point Road, Groton, CT, 06340, US

(21) Appl. No.: 09/895,367

(22) Filed: Jun. 29, 2001

Related U.S. Application Data
(60) Provisional application No. 60/219,100, filed on Jul. 18, 2000.
(60) Provisional application No. 60/265,358, filed on Jan. 31, 2001.

(30) Foreign Application Priority Data
Jul. 6, 2000 (GB) 0016684.3
Dec. 15, 2000 (GB) 0030647.2
Mar. 13, 2001 (GB) 0106167.0
Apr. 4, 2001 (GB) 0108483.9

(51) Int. Cl. A61K 31/5377; A61K 31/496; A61K 31/53; A61K 31/519

(52) U.S. Cl. 514/210.21; 514/234.2; 514/246; 514/252.16; 514/258; 514/362

(57) ABSTRACT

The present invention relates to the use of neutral endopeptidase inhibitors (NEPi) and a combination of NEPi and phosphodiesterase type 5 (PDE5) inhibitor for the treatment of male sexual dysfunction, in particular MED.

This application claims priority from provisional application U.S. serial No. 60/219,100 filed Jul. 19, 2000 and from provisional application U.S. serial No. 60/265,358 filed Jan. 31, 2001 the benefit of which is hereby claimed under 37 C.F.R.§1 .78(a)(3).

### FIELD OF INVENTION

The present invention relates to compounds and pharmaceutical compositions for use in the treatment of male sexual dysfunction, in particular male erectile dysfunction (MED). Male sexual function as referred to herein is meant to include ejaculatory disorders such as premature ejaculation, or anorgasmia (unable to achieve orgasm), desire disorders such as hypoactive sexual desire disorder (lack of interest in sex).

The present invention also relates to a method of treatment of MED.

The present invention also relates to assays to screen for the compounds of the present invention and which form part of the pharmaceutical compositions of the present invention and which are useful in the treatment of male sexual dysfunction, in particular MED.

For convenience, a list of abbreviations that are used in the following text is presented before the Claims section.

### SEXUAL DYSFUNCTION

Sexual dysfunction (SD) is a significant clinical problem which can affect both males and females. The causes of SD may be both organic as well as psychological. Organic aspects of SD are typically caused by underlying vascular diseases, such as those associated with hypertension or diabetes mellitus, by prescription medication and/or by psychiatric disease such as depression. Physiological factors include fear, performance anxiety and interpersonal conflict. SD impairs sexual performance, diminishes self-esteem and disrupts personal relationships thereby inducing personal distress. In the clinic, SD disorders have been divided into female sexual dysfunction (FSD) disorders and male sexual dysfunction (MSD) disorders (Melman et al 1999). FSD is best defined as the difficulty or inability of a woman to find satisfaction in sexual expression. Male sexual dysfunction (MSD) is generally associated with erectile dysfunction, also known as male erectile dysfunction (MED) (Benet et al 1994).

### MALE ERECTILE DYSFUNCTION (MED)

It is known that some individuals can suffer from male erectile dysfunction (MED). Male erectile dysfunction (MED) is defined as:

“the inability to achieve and/or maintain a penile erection for satisfactory sexual performance” (NIH Consensus Development Panel on Impotence, 1993)”

It has been estimated that the prevalence of erectile dysfunction (ED) of all degrees (minimal, moderate and complete impotence) is 52% in men 40 to 70 years old, with higher rates in those older than 70 (Melman et al 1999). The condition has a significant negative impact on the quality of life of the patient and their partner, often resulting in increased anxiety and tension which leads to depression and low self esteem. Whereas two decades ago, MED was primarily considered to be a psychological disorder (Benet et al 1994), it is now known that for the majority of patients there is an underlying organic cause. As a result, much progress has been made in identifying the mechanism of normal penile erection and the pathophysiology of MED.

Penile erection is a haemodynamic event which is dependent upon the balance of contraction and relaxation of the corpus cavernosal smooth muscle and vasculature of the penis (Lerner et al 1993). Corpus cavernosal smooth muscle is also referred to herein as corporal smooth muscle or in the plural sense corpus cavernosa. Relaxation of the corpus cavernosal smooth muscle leads to an increased blood flow into the trabecular spaces of the corpus cavernosa, causing them to expand against the surrounding tunica and compress the draining veins. This produces a vast elevation in blood pressure which results in an erection (Naylor, 1998).

The changes that occur during the erectile process are complex and require a high degree of coordinated control involving the peripheral and central nervous systems, and the endocrine system (Naylor, 1998). Corporal smooth muscle contraction is modulated by sympathetic noradrenergic innervation via activation of postsynaptic α₁ adrenoceptors. MED may be associated with an increase in the endogenous smooth muscle tone of the corpus cavernosum. However, the process of corporal smooth muscle relaxation is mediated partly by non-adrenergic, non-cholinergic (NANC) neurotransmission. There are a number of other NANC neurotransmitters found in the penis, other than NO, such as calcitonin gene related peptide (CGRP) and vasoactive intestinal peptide (VIP). The main relaxing factor responsible for mediating this relaxation is nitric oxide (NO), which is synthesised from L-arginine by nitric oxide synthase (NOS) (Taub et al 1993; Chuang et al 1998). It is thought that reducing corporal smooth muscle tone may aid NO to induce relaxation of the corpus cavernosum. During sexual arousal in the male, NO is released from neurones and the endothelium and binds to and activates soluble guanylate cyclase (sGC) located in the smooth muscle cells and endothelium, leading to an elevation in intracellular cyclic guanosine 3′,5′-monophosphate (cGMP) levels. This rise in cGMP leads to a relaxation of the corpus cavernosum due to a reduction in the intracellular calcium concentration ([Ca²⁺]ᵢ), via unknown mechanisms thought to involve protein kinase G activation (possibly due to activation of Ca²⁺ pumps and Ca²⁺-activated K⁺ channels; Chuang et al., 1998).

Sildenafil citrate (also known as Viagra™) has recently been developed by Pfizer as the first oral drug treatment for MED. Sildenafil acts by inhibiting cGMP breakdown in the corpus cavernosa by selectively inhibiting phosphodiesterase 5 (PDE5), thereby limiting the hydrolysis of cGMP to 5′GMP (Boolel et al., 1996; Jeremy et al., 1997) and thereby increasing the intracellular concentrations of cGMP and facilitating corpus cavernosal smooth muscle relaxation.

Currently, all other available MED therapies on the market, such as treatment with prostaglandin based compounds i.e. alprostadil which can be administered intra-urethrally (available from Vivus Inc., as Muse™) or via small needle injection (available from Pharamcia & Upjohn, as Caverject™), are either inconvenient and/or invasive. Other treatments include vacuum constriction devices, vasoactive drug injection or penile prostheses implantation (Montague et al., 1996). Although injectable vasoactive drugs show high efficacy, side effects such as penile pain, fibrosis and priapism are common, and injection therapy is not as convenient as oral therapy therefore sildenafil currently represents the most preferred therapy on the market.

### REFERENCES

Argiolas, A. et al (1995), Neuromodulation of penile erection. Prog Neurobiol. 47: 235-255.

Benet, A. E. et al (1994), Male erectile dysfunction assessment and treatment options. Comp. Ther. 20: 669-673.

Boolel, M. et al (1996). Sildenafil, a novel effective oral therapy for male erectile dysfunction. Br. J. of Urology 78: 257-261.

Carter A J. et al (1998). Effect of the selective phosphodiesterase type 5 inhibitor sildenafil on erectile dysfunction in the anesthetized dog. J. Urol. 160: 242-6.

Chiou, W. F. et al (1998). Relaxation of corpus cavernosum and raised intracavernous pressure by berberine in rabbit. Br. J. Pharmacol. 125: 1677-1684.

Jeremy, J. Y. et al (1997). Effects of sildenafil, a type-5 cGMP phosphodiesterase inhibitor, and papaverine on cyclic GMP and cyclic AMP levels in the rabbit corpus cavernosum in vitro. Br. J. Urology 79: 958-963.

Lerner, S. E. et al (1993). A review of erectile dysfunction: new insights and more questions. J. Urology 149: 1246-1255.

Melman, A. & Gingell, J. C. (1999). The epidemiology and pathophysiology of erectile dysfunction. J. Urology 161:5-11.

Montague, D. et al (1996). Clinical guidelines panel on erectile dysfunction: Summary report on the treatment of organic erectile dysfunction. J. Urology 156: 2007-2011.

Naylor, A. M. (1998). Endogenous neurotransmitters mediating penile erection. Br. J. Urology 81: 424-431.

NIH Consensus Development Panel on Impotence (1993). NIH Consensus Conference Impotence. J.A.M.A. 270: 83.

Omote M. (1999). Pharmacological profiles of sildenafil (VIAGRA) in the treatment of erectile dysfunction: efficacy and drug interaction with nitrate. Nippon Yakurigaku Zasshi. 114:213-8.

Taub, H. C. et al (1993). Relationship between contraction and relaxation in human and rabbit corpus cavernosum. Urology 42: 698-704.

Traish A M, et al (1999). Effects of castration and androgen replacement on erectile function in a rabbit model. Endocrinology. 140: 1861-8.

Turner, A J. et al (1997). Mammalian membrane metallopeptidases: NEP, ECE, KELL and PEX. FASEB J. 11: 355-364.

### SUMMARY ASPECTS OF THE PRESENT INVENTION

A seminal finding of the present invention is the ability to treat an male suffering from sexual dysfunction, in particular MED, with use of a neutral endopeptidase inhibitor (NEPi). Surprisingly the applicants have also found that inhibition of NEP EC3.4.24.11 with a neutral endopeptidase inhibitor, hereinafter referred to as an NEPi, significantly enhances the nerve-stimulated erectile process.

According to the present invention there is provided the use of an inhibitor of the neutral endopeptidase EC3.4.24.11, for the treatment of male sexual dysfunction, in particular MED.

Preferably, the NEP inhibitors for use in the treatment of male sexual dysfunction, in particular MED according to the present invention have an IC₅₀ at less than 100 nanomolar, more preferably, at less than 50 nanomolar.

Preferably, the NEP inhibitors according to the present invention have greater than 100-fold, more preferably greater than 300-fold selectivity for NEP over angiotensin converting enzyme (ACE). This reduces the prospect of cardiovascular events (e.g. drop in blood pressure) when the NEPi is administered systemically (e.g. by mouth). Preferably the NEPi also has a greater than 100 fold selectivity over endothelin converting enzyme (ECE).

There is further provided the use of a NEPi in the manufacture of a medicament for the treatment of MED.

There is no documented evidence for the expression or a functional role of NEP EC3.4.24.11 in the penis or corpus cavernosum or in the erectile mechanism/process.

There is also no documented evidence for a functional or biochemical effect for NEP inhibitors on the penis or corpus cavernosum or alternatively in the erectile mechanism/process.

In particular the present invention provides NEPi compounds for use in the treatment of MED.

The present invention is advantageous as it provides a means for restoring a normal sexual arousal response—namely increased penile blood flow leading to erection of the penis. Hence, the present invention provides a means to restore, or potentiate, the normal sexual arousal response.

Some NEPi compounds were prepared according to the teachings presented in the Experimental section (infra). They were tested as agents and were found to be useful for enhancing the endogenous erectile process, and thereby being useful in the treatment of MED. Some of the experimental data concerning the NEPi are presented in the Experimental section (infra).

Without being limited to any particular theory it is proposed herein that by inhibiting NEP EC3.4.24.11 other neuronally released vasoactive agents that are released during sexual arousal are enhanced, most likely vasoactiveintestinal protein (VIP). It is believed that use of the NEPi potentiates the effects of neuropeptides most likely (VIP) that are released during sexual stimulation, and hence potentiates the erectile mechanism by increasing cavernosal blood flow and thus intracavernosal pressure.

It is further proposed that the use of the compounds according to the present invention acts via enhancing a non-NO dependant NANC pathway to treat MED, and to potentiate or facilitate the nitrergic signalling in the penis.

Surprisingly the applicants have also found that inhibition of NEP with a NEPi, significantly potentiates PDE5 inhibitor-mediated enhancement of the erectile process.

Since NEP is present throughout the body, it is very unexpected NEPi can be administered systemically and achieve a therapeutic response in the male genitalia witout provoking intolerable (adverse) side effects. Thus in the in vivo (rabbit) results hereafter the NEPi alone (particularly having a selectivity as above) and NEPi/PDE5 combination when administered systemitcally increased genital blood flow, upon sexual arousal (mimiced by pelvic nerve stimulation) without adversely affecting cardiovascular parameters, such as causing a significant hypotensive or hypertensive effects (see FIG. 6 hereinafter).

Thus according to a preferred aspect of the invention, there is provided the use of a NEPi by systemic administraiton (preferably by mouth e.g. swallowable tablet or capsule, or a sublingual or buccal formulation) in the prepartion of a medicament for the treatment of male sexual dysfunction, in particular MED.

Thus according to a further embodiment the present invention provides the use of one or more NEPi's and one or more PDE5i's for the treatment male sexual dysfunction, in particular MED. Other combinations in accordance with the present inventions are disclosed hereinafter.

Preferably said combined treatment comprises a combination of one or more NEPi's with one or more PDE5i's. More preferably such combination provides for the concomitant administration of one or more NEPi's with one or more PDE5I's for the treatment of MED.

Highly preferred herein is the use of a pharmaceutical composition comprising one or more NEPi's with one or more PDE5i's for the treatment of MED.

Our results show that suprisingly this combination can be given systemically (preferably by mouth e.g. a swallowable tablet or capsule, sublingual or bucal formulatation) with minimal drop in blood pressure—thus allowing systemic treatment of male sexual dysfunction using the combination.

Especially preferred for use in the pharmaceutical compositions for the treatment of MED according to the present invention is the combination of a potent and selective NEPi with a potent and selective PDE5i. Preferred values for these are given hereinafter together with a screening methods for determining the values.

In a preferred embodiment herein said combined administration of NEPi and PDE5i is concomitant. Concomitant administration as defined herein encompasses simultaneous (separate) administration, simultaneous combined administration, separate administration, combined administration, sequential administration and co-formulated combined administration of a PDE5i and a NEPi.

As detailed hereinbefore the present invention further proposes that, concomitant administration of a PDE5i and NEPi can effect an increase in the efficacy as compared with that obtainable by PDE5-alone associated MED therapy. For example and discussion thereof see Test Results Section, Examples 4 and 5.

According to a further aspect of the present invention it is proposed herein that, concomitant application of an NEPi and a PDE5i can provide faster onset of action that that achievable via the PDE5i alone. In other words the present invention additionally provides the use of a fast-acting composition for the treatment of MED. A fast acting MED composition as defined herein, and as exemplified hereinafter, means that following i.v. administration of the composition (NEPi and PDE5i) the time to maximal effect on intracavernosal pressure is reduced versus the equivalent time obtained for the same dose of the PDE5i alone. For example and discussion thereof see Test Results Section, Examples 5

Thus, a further aspect of the invention provides a fast acting pharmaceutical compositions comprising an NEPi and a PDE5i for use in the treatment of MED.

It is further proposed herein that use of a NEPi/PDE5i combination may enhance the efficacy of the PDE5i thereby enabling a reduction in the dose of PDE5 inhibitor required for a specific efficacy. A formulation comprising a NEPi and a reduced amount of a PDE5i as defined herein means that a reduced amount of a given PDE5i is required to effect a particular response when combined with an effective amount of a NEPi according to the present invention than the required amount of PDE5i alone. Such reduced dose compositions for the treatment of MED reduce the potential nitrate interactions of PDE5. Furthermore it may be desirable for particular patient groups such as for example men with mild MED. This may be particularly advantageous to patients who respond poorly to a PDE5 inhibitor alone (e.g. sildenafil)—as illustrated in examples 4 and 5.

### Patient Groups

Patients with mild to moderate MED should benefit from treatment with a NEPi, and patients with severe MED may also respond. However, early investigations suggest that the responder rate of patients with mild, moderate and severe MED will be greater with a NEP/PDE5 inhibitor combination. Mild, moderate and severe MED will be terms known to the man skilled in the art, but guidance can be found in: The Journal of Urology, vol 151, 54-61 (Jan 1994).

Early investigations suggest the below mentioned MED patient groups should benefit from treatement with a NEPi and a PDE5i (or other combination set out hereinafter). These patient groups which are described in more detail in Clinical Andrology vol 23,no.4, p773-782, and chapter 3 of the book by 1. Eardley and K. Sethia “Erectile Dysfunction—Current Investigation and Management, published by Mosby-Wolfe are as follows: psycogenic, endocrinologic, neurogenic, arteriogenic, drug-induced sexual dysfunction (lactogenic) and sexual dysfunction related to cavernosal factors, particularly venogenic causes.

### NEP EC3.4.24.11

NEP EC3.4.24.11, also known as enkephalinase or neprilysin, is a zinc-dependent neutral endopeptidase. This enzyme is involved in the breakdown of several bioactive oligopeptides, cleaving peptide bonds on the amino side of hydrophobic amino acid residues (Reviewed in Turner et al., 1997). The key neuronally released bioactive agents or neuropeptides metabolised by NEP include natriuretic peptides such as atrial natriuretic peptides (ANP) as well as brain natriuretic peptide and C-type natriuretic peptide, bombesin, bradykinin, calcitonin gene-related peptide, endothelins, enkephalins, neurotensin, substance P and vasoactive intestinal peptide. Some of these peptides have potent vasodilatory and neurohormone functions, diuretic and natriuretic activity or mediate behaviour effects.

Background teachings on NEP have been presented by Victor A. McKusick et al on http://www3.ncbi.nim.nih.gov/Omim/searchomim.htm. The following information concerning NEP has been extracted from that source.

“Common acute lymphocytic leukemia antigen is an important cell surface marker in the diagnosis of human acute lymphocytic leukemia (ALL). It is present on leukemic cells of pre-B phenotype, which represent 85% of cases of ALL. CALLA is not restricted to leukemic cells, however, and is found on a variety of normal tissues. CALLA is a glycoprotein that is particularly abundant in kidney, where it is present on the brush border of proximal tubules and on glomerular epithelium. Letarte et al. (1988) cloned a cDNA coding for CALLA and showed that the amino acid sequence deduced from the cDNA sequence is identical to that of human membrane-associated neutral endopeptidase (NEP; EC 3.4.24.11), also known as enkephalinase. NEP cleaves peptides at the amino side of hydrophobic residues and inactivates several peptide hormones including glucagon, enkephalins, substance P, neurotensin, oxytocin, and bradykinin. By cDNA transfection analysis, Shipp et al. (1989) confirmed that CALLA is a functional neutral endopeptidase of the type that has previously been called enkephalinase. Barker et al. (1989) demonstrated that the CALLA gene, which encodes a 100-kD type 11 transmembrane glycoprotein, exists in a single copy of greater than 45 kb which is not rearranged in malignancies expressing cell surface CALLA. The gene was located to human chromosome 3 by study of somatic cell hybrids and in situ hybridization regionalized the location to 3q21-q27. Tran-Paterson et al. (1989) also assigned the gene to chromosome 3 by Southern blot analysis of DNA from human-rodent somatic cell hybrids. D'Adamio et al. (1989) demonstrated that the CALLA gene spans more than 80 kb and is composed of 24 exons.

### REFERENCES FOR THE NEP SECTION

1. Barker, P. E.; Shipp, M. A.; D'Adamio, L.; Masteller, E. L.; Reinherz, E. L. The common acute lymphoblastic leukemia antigen gene maps to chromosomal region 3(q21-q27). J. Immun. 142: 283-287, 1989.

2. D'Adamio, L.; Shipp, M. A.; Masteller, E. L.; Reinherz, E. L.: Organization of the gene encoding common acute lymphoblastic leukemia antigen (neutral endopeptidase 24.11): multiple miniexons and separate 5-prime untranslated regions. Proc. Nat. Acad. Sci. 86: 7103-7107, 1989.

3. Letarte, M.; Vera, S.; Tran, R.; Addis, J. B. L.; Onizuka, R. J.; Quackenbush, E. J.; Jongeneel, C. V.; McInnes, R. R.: Common acute lymphocytic leukemia antigen is identical to neutral endopeptidase. J. Exp. Med. 168: 1247-1253, 1988.

4. Shipp, M. A.; Vijayaraghavan, J.; Schmidt, E. V.; Masteller, E. L.; D'Adamio, L.; Hersh, L. B.; Reinherz, E. L.: Common acute lymphoblastic leukemia antigen (CALLA) is active neutral endopeptidase 24.11 (‘enkephalinase’): direct evidence by cDNA transfection analysis. Proc. Nat. Acad. Sci. 86: 297-301, 1989.

5. Tran-Paterson, R.; Willard, H. F.; Letarte, M.: The common acute lymphoblastic leukemia antigen (neutral endopeptidase—3.4.24. 11) gene is located on human chromosome 3. Cancer Genet.Cytogenet.42:129-134, 1989.

### DETAILED ASPECTS OF THE PRESENT INVENTION

In one aspect, the present invention relates to NEPi compounds and pharmaceutical compositions including NEPi compounds and pharmaceutical combinations comprising NEPi and PDE5i for use (or when in use) in the treatment of male sexual dysfunction, in particular MED. In said pharmaceutical compositions the NEPi (and PDE5I, if present, and/or additional agent) is optionally admixed with a pharmaceutically acceptable carrier, diluent or excipient. Here, the composition (like any of the other compositions mentioned herein) may be packaged for subsequent use in the treatment of male sexual dysfunction, in particular MED.

In another aspect, the present invention relates to the use of an agent in the manufacture of a medicament (such as a pharmaceutical composition) for the treatment of male sexual dysfunction, in particular MED.

In a further aspect, the present invention relates to a method of treating a male suffering from male sexual dysfunction, in particular MED; the method comprising delivering to the male an NEPi that is capable of enhancing the endogenous erectile process in the corpus cavernosum; wherein the NEPi is present in an amount to enhance the endogenous erectile process as defined hereinbefore; wherein the NEPi is optionally admixed with a pharmaceutically acceptable carrier, diluent or excipient; and wherein said NEPi is as herein defined.

In a further aspect, the present invention relates to an assay method for identifying an agent (hereinafter referred to as a NEPi) that can be used to treat male sexual dysfunction, in particular MED, the assay method comprising: determining whether a test agent can directly enhance the endogenous erectile process; wherein said enhancement is defined as a potentiation of intracavernosal pressure (ICP) (and/or cavernosal blood flow) in the presence of a test agent as defined herein; such potentiation by a test agent is indicative that the test agent may be useful in the treatment of male sexual dysfunction, in particular MED and wherein said test agent is a NEPi.

By way of example, the present invention relates to an assay method for identifying an agent that can directly enhance the endogenous erectile process in order to treat male sexual dysfunction, in particular MED, the assay method comprising: contacting a test agent which has a moeity capable of inhibiting the metabolic breakdown of a peptide (preferably a fluorescent labelled peptide), said peptide being normally metabolised by NEP; and measuring the activity and/or levels of peptide remaining after a fixed time (for example via fluorometric analysis); wherein the change in the level of the peptede (e.g) measured by fluorescence is indicative of the potency (IC₅₀) of the test agent and is indicative that the test agent may be useful in the treatment of male sexual dysfunction, in particular MED; and wherein said agent is an NEPi.

In a further aspect, the present invention relates to a process comprising the steps of: (a) performing the assay according to the present invention; (b) identifying one or more agents that can directly enhance the endogenous erectile process; and (c) preparing a quantity of those one or more identified agents; and wherein said agent is an NEPi.

With this aspect, the agent identified in step (b) may be modified so as to, for example, maximise activity and then step (a) may be repeated. These steps may be repeated until the desired activity or pharmacokinetic profile has been achieved.

Thus, in a further aspect, the present invention relates to a process comprising the steps of: (a1) performing the assay according to the present invention; (b1) identifying one or more agents that can directly enhance the endogenous erectile process; (b2) modifying one or more of said identified agents; (a2) optionally repeating step (a1); and (c) preparing a quantity of those one or more identified agents (i.e. those that have been modified); and wherein said agent is an NEPi.

In a further aspect, the present invention relates to a method of treating male sexual dysfunction, in particular MED, by potentiating the nerve stimulated endogenous erectile process in vivo (rabbit and/or dog) by measuring the ICP or cavernosal blood flow with an agent; wherein the agent is capable of directly inhibiting the metabolic breakdown of a fluorescent peptide (as detailed hereinbefore) in an in vitro assay method; wherein the in vitro assay method is the assay method according to the present invention; and wherein said agent is an NEPi.

In a further aspect, the present invention relates to the use of an agent in the preparation of a pharmaceutical composition for the treatment of male sexual dysfunction, in particular MED, wherein the agent is capable of directly inhibiting the metabolic breakdown of a fluorescent peptide when assayed in vitro by the assay method according to the present invention; and wherein said agent is an NEPi.

In a further aspect, the present invention relates to an animal model used to identify agents capable of treating male sexual dysfunction (in particular MED), said model comprising an anaesthetised male animal including means to measure changes in intracavernosal pressure and/or cavernosal blood flow of said animal following stimulation of the pelvic nerve thereof; and wherein said agent is an NEPi.

In a further aspect, the present invention relates to an assay method for identifying an agent that can directly enhance the endogenous erectile process in order to treat MED, the assay method comprising: administering an agent to the animal model of the present invention; and measuring the change in the endogenous erectile process; wherein said change is defined as a potentiation of intracavernosal pressure (ICP) (and/or cavernosal blood flow) in the animal model in the presence of a test agent as defined; and wherein said agent is an NEPi.

In a further aspect, the present invention relates to a diagnostic method, the method comprising isolating a sample from a male; determining whether the sample contains an entity present in such an amount as to cause male sexual dysfunction, preferably MED; wherein the entity has a direct effect on the endogenous erectile process in the corpus cavernosum of the male; and wherein said entity can be modulated to achieve a beneficial effect by use of an agent; and wherein said agent is an NEPi.

In a further aspect, the present invention relates to a diagnostic composition or kit comprising means for detecting an entity in an isolated male sample; wherein the means can be used to determine whether the sample contains the entity and in such an amount to cause male sexual dysfunction, preferably MED, or is in an amount so as to cause sexual dysfunction, preferably MED; wherein the entity has a direct effect on the endogenous erectile process and wherein said entity can be modulated to achieve a beneficial effect by use of an agent; and wherein said agent is an NEPi.

For ease of reference, these and further aspects of the present invention are now discussed under appropriate section headings. However, the teachings under each section are not necessarily limited to each particular section.

### PREFERABLE ASPECTS

The agents for use in the treatment of MED according to the present invention are NEP EC3.4.24.11 inhibitors.

In one embodiment, preferably the agent for the use according to the present invention may be used via oral administration.

In another embodiment, the agent for the use according to the present invention may be used via topical application to the penis or intra-urethral administration.

For some applications, preferably the agent for the use according to the present invention is a selective NEPi.

Preferably the agent for use in the treatment of MED according to the present invention is an inhibitor—i.e. it is capable of exhibiting an inhibitory function.

Preferably the agent for use in the treatment of MED according to the present invention is capable of directly enhancing the endogenous erectile process as detailed hereinbefore.

### Preferred NEPi

Preferred for use as NEPi in accordance with the invention are compounds of the general formula 1 (as disclosed in co-pending application nos GB 0101584 and U.S. 60/274957 filed Mar. 12, 2001):

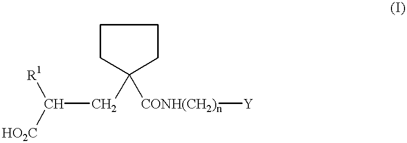

wherein

R¹ is C₁₋₆alkyl which may be substituted by one or more substituents, which may be the same or different, selected from the list: halo, hydroxy, C₁₋₆ alkoxy, C₂₋₆ hydroxyalkoxy, C₁₋₆alkoxy(C₁₋₆alkoxy), C₃₋₇cycloalkyl, C₃₋₇cycloalkenyl, aryl, aryloxy, (C₁₋₄alkoxy)aryloxy, heterocyclyl, heterocyclyloxy, —NR²R³, —NR⁴COR⁵, —NR⁴SO₂R⁵, —CONR²R³, —S(O)ₚR⁶, —COR⁷ and —CO₂(C₁₋₄alkyl); or R¹ is C₃₋₇cycloalkyl, aryl or heterocyclyl, each of which may be substituted by one or more substituents from said list, which substituents may be the same or different, which list further includes C₁₋₆alkyl; or R¹ is C₁₋₆alkoxy, —NR²R³ or —NR⁴SO₂R⁵;

wherein

R² and R³ are each independently H, C₁₋₄alkyl, C₃₋₇cycloalkyl (optionally substituted by hydroxy or C₁₋₄alkoxy), aryl, (C₁₋₄alkyl)aryl,

C₁₋₆alkoxyaryl or heterocyclyl; or R² and R³ together with the nitrogen to which they are attached form a pyrrolidinyl, piperidino, morpholino, piperazinyl or N-(C₁₋₄alkyl)piperazinyl group;

R⁴ is H or C₁₋₄alkyl;

R⁵is C₁₋₄alkyl, CF₃, aryl, (C₁₋₄alkyl)aryl, (C₁₋₄alkoxy)aryl, heterocyclyl, C₁₋₄alkoxy or —NR²R³ wherein R² and R³ are as previously defined;

R⁶ is C₁₋₄alkyl, aryl, heterocyclyl or NR²R³ wherein R² and R³ are as previously defined; and

R⁷ is C₁₋₄alkyl, C₃₋₇cycloalkyl, aryl or heterocyclyl; n is 0, 1 or 2; p is 0, 1, 2 or 3;

the —(CH₂)ₙ— linkage is optionally substituted by C₁₋₄alkyl, C₁₋₄alkyl substituted with one or more fluoro groups or phenyl, C₁₋₄alkoxy, hydroxy, hydroxy(C₁₋₃alkyl), C₃₋₇cycloalkyl, aryl or heterocyclyl;

Y is the group

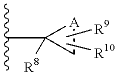

wherein A is —(CH₂)_{q}— where q is 1, 2, 3 or 4 to complete a 3 to 7 membered carbocyclic ring which may be saturated or unsaturated; R⁸ is H, C₁₋₆alkyl, —CH₂OH, phenyl, phenyl(C₁₋₄alkyl) or CONR¹¹R¹²; R⁹ and R¹⁰ are each independently H, —CH₂OH, —C(O)NR¹¹R¹², C₁₋₆alkyl, phenyl (optionally substituted by C₁₋₄alkyl, halo or C₁₋₄alkoxy or phenyl(C₁₋₄alkyl) wherein the phenyl group is optionally substituted by C₁₋₄alkyl, halo or C₁₋₄alkoxy, or R⁹ and R¹⁰ together form a dioxolane; R¹¹ and R¹² which may be the same or different are H, C₁₋₄alkyl, R¹³ or S(O)ᵣR¹³, where r is 0, 1 or 2 and R¹³ is phenyl optionally substituted by C₁₋₄alkyl or phenylC₁₋₄alkyl wherein the phenyl is optionally substituted by C₁₋₄alkyl; or

Y is the group, —C(O) NR¹¹R¹² wherein R¹¹ and R¹² are as previously defined except that R¹¹ and R¹² are not both H; or

Y is the group,

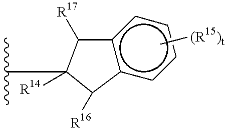

wherein R¹⁴ is H, CH₂OH, or C(O)NR¹¹R¹² wherein R¹¹ and R¹² are as previously defined; when present R¹⁵, which may be the same or different to any other R¹⁵, is OH, C₁₋₄alkyl, C₁₋₄alkoxy, halo or CF₃; t is 0,1, 2, 3 or 4; and R¹⁶ and R¹⁷ are independently H or C₁₋₄alkyl; or

Y is the group

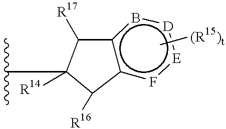

wherein one or two of B, D, E or F is a nitrogen, the others being carbon; and R¹⁴ to R¹⁷ and t are as previously defined; or

Y is an optionally substituted 5-7 membered heterocyclic ring, which may be saturated, unsaturated or aromatic and contains a nitrogen, oxygen or sulphur and optionally one, two or three further nitrogen atoms in the ring and which may be optionally benzofused and optionally substituted by:

C₁₋₆alkoxy; hydroxy; oxo; amino; mono or di-(C₁₋₄alkyl)amino; C₁₋₄alkanoylamino; or

C₁₋₆alkyl which may be substituted by one or more substituents, which may be the same or different, selected from the list: C₁₋₆alkoxy, C₁₋₆haloalkoxy, C₁₋₆alkylthio, halogen, C₃₋₇cycloalkyl, heterocyclyl or phenyl; or

C₃₋₇cycloalkyl, aryl or heterocyclyl, each of which may be substituted by one or more substituents, which may be the same or different, selected from the list: C₁₋₆alkyl, C₁₋₆alkoxy, C₁₋₆haloalkoxy, C₁₋₆alkylthio, halogen, C₃₋₇cycloalkyl, heterocyclyl or phenyl;

wherein when there is an oxo substitution on the heterocyclic ring, the ring only contains one or two nitrogen atoms and the oxo substitution is adjacent a nitrogen atom in the ring; or

Y is —NR¹⁸S(O)ᵤR¹⁹, wherein R¹⁸ is H or C₁₋₄alkyl; R¹⁹ is aryl, arylC₁₋₄alkyl or heterocyclyl (preferably pyridyl); and

us is 0, 1,2 or 3.

Particularly preferred compounds of the invention are:

2-[(1-{[(1-benzyl-6-oxo-1,6-dihydro-3-pyridinyl)amino]carbonyl}cyclopentyl)-methyl]-4-methoxybutanoic acid (Example 6);

2-{[1-({[3-(2-oxo-1-pyrrolidinyl)propyl]amino}carbonylcyclopentyl]-methyl}-4-phenylbutanoic acid (Example 7);

(+)-2-{[1-({[2-(hydroxymethyl)-2,3-dihydro-1H-inden-2-yl]amino}carbonyl)cyclopentyl]methyl}-4-phenylbutanoic acid (Example 8);

2-[(1-{[(5-methyl-1,3,4-thiadiazol-2-yl)amino]carbonyl}cyclopentyl)methyl]-4-phenylbutanoic acid (Example 10);

cis-3-(2-methoxyethoxy)-2-[(1-{[(4-{[(phenylsulfonyl)amino]carbonyl}cyclohexyl)-amino]carbonyl}cyclopentyl)methyl]propanoic acid (Example 11);

(+)-2-{[1-({[2-(hydroxymethyl)-2,3-dihydro-1H-inden-2-yl]amino}carbonyl)cyclopentyl]-methyl}pentanoic acid (Example 12);

(2R)-2-[(1-{[(5-ethyl-1,3,4-thiadiazol-2-yl)amino]carbonyl}cyclopentyl) methyl]pentanoic acid or (−)-2-[(1-{[(5-ethyl-1,3,4-thiadiazol-2-yl)amino]carbonyl}cyclopentyl)methyl]pentanoic acid (Example 1);

(2S)-2-[(1-{[(5-ethyl-1,3,4-thiadiazol-2-yl)amino]carbonyl}cyclopentyl)-methyl]pentanoic acid or (+)-2-[(1-{[(5-ethyl-1,3,4-thiadiazol-2-yl)amino]carbonyl}cyclopentyl)-methyl]pentanoic acid (Example 2); and

(S)-2-{[1-({[2-(hydroxymethyl)-2,3-dihydro-1H-inden-2-yl]amino}carbonyl)-cyclopentyl]methyl}-4-methoxybutanoic acid (Example 4).

### General Routes

Compounds of the invention may be prepared, in known manner, in a variety of ways.

Throughout the specification, general formulae are designated by Roman numerals I, II, III, IV etc. Subsets of these general formulae are defined as Ia, Ib, Ic etc, . . . IVa, IVb, IVc etc.

Compounds of general formula I may be prepared according to reaction scheme 1, by reacting a compound of formula II (where Prot is a suitable protecting group) with a primary amine of formula III to give a compound of formula IV. Deprotection gives compounds of formula I.

Preferred reaction conditions for the acid/amine coupling step comprise reacting 11 with III (or its amine salt) in the presence of an activating agent, optionally a catalyst, and an excess of an acid acceptor, in a suitable solvent. Particularly preferred reaction conditions comprise reacting II (1-1.5 equivalents), III (or its salt 1-1.5 equivalents), in the presence of 1-(3-dimethylaminopropyl)-3-ethylcarbodiimide hydrochloride (WSCDI) or N,N′-dicyclohexylcarbodiimide (DCC) (1.1-1.3 equivalents), 1-hydroxybenzotrazole hydrate (HOBT) or dimethylaminopyridine (DMAP) (1.05-1.2 equivalents), N-methyl morpholine (NMM) or triethyamine (2.3-3 equivalents), in dimethylformamide or dichloromethane at between room temperature and 90° C. for 16-18 hours.

Alternatively, the acid/amine coupling step may be prepared via the acid chloride in the presence of an excess of acid acceptor, in a suitable solvent. The acid chloride may be isolated or it may be generated in situ. Preferred reaction conditions comprise reacting the acid chloride of II (1-1.1 equivalents), III (or its salt, 1 to 1.5 equivalents), triethyamine or N-methyl morpholine (1.4-10 equivalents), in dichloromethane at room temperature for 24 hours. Compounds of formula II can be converted to the acid chloride in situ by treatment with oxalyl chloride in dichloromethane in the presence of a catalytic amount of dimethylformamide for 2 hours at room temperature.

Methods for deprotection of an acid group depend on the protecting group. For examples of protection/deprotection methodology see “Protective groups in Organic synthesis”, T W Greene and P G M Wutz.

For example, when Prot is a tert-butyl, deprotection conditions comprise reacting IV with trifluoroacetic acid/dichloromethane (1:1-1.5 by volume), at room temperature for 2-18 hours, optionally in the presence of a carbocation scavenger, e.g. anisole (10 equivalents). When Y contains a hydroxy group, base hydrolysis of the intermediate trifluoroacetic acid ester may be necessary. Alternative methodology for deprotection when Prot is tert-butyl comprises treating IV with hydrochloric acid in dichloromethane at room temperature for 3 hours. For the avoidance of doubt, Prot as tert-butyl is given by way of Example and is not intended to be limited to tert-Butyl.

When Prot is benzyl, deprotection conditions comprise reacting IV with palladium on charcoal (5-10%) in aqueous ethanol (40-95%) at 15-60 psi at room temperature for 2 hrs to 3 days.

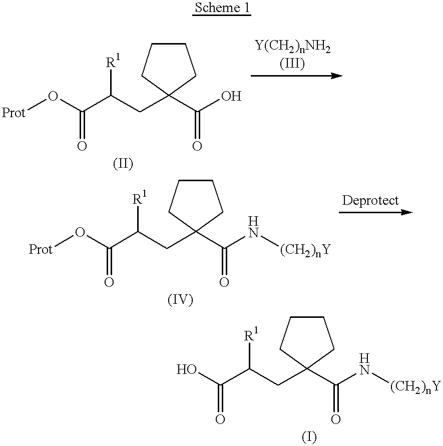

Compounds of formula Ia, i.e. compounds of general formula I where Y is —NHSO₂R¹⁹, may be prepared according to reaction scheme 2. Compounds of formula V are first prepared by reacting compounds of formula II with compounds of formula VI where Prot² is a suitable amine protecting group. Preferred reaction conditions are analogous to those described the acid/amine coupling step for Scheme 1 above. Selective amine deprotection of compounds of formula V gives compounds of formula VII. Compounds of formula VII are reacted with R¹⁹SO₂Cl in the presence of an acid acceptor in a suitable solvent to form compounds of formula VIII. Deprotection of compounds of formula VIII under analogous conditions to those described for the deprotection step of Scheme 1 gives compounds of formula Ia.

Methods for deprotection of an amine group depend on the protecting group. For examples of protection/deprotection methodology see “Protective groups in Organic Synthesis”, T W Greene and P G M Wutz. For example, when Prot² is benzoyloxycarbonyl, deprotection conditions comprise reacting V with palladium on charcoal (10%) in ethanol at room temperature for 18 hours.

Preferred methods for preparation of the compounds of formula VIII comprise reaction of VII with R¹⁹SO₂Cl (1 equivalent) in the presence of triethyamine (1.5-2.5 equivalents) in dichloromethane at room temperature for 2 to 3 days.

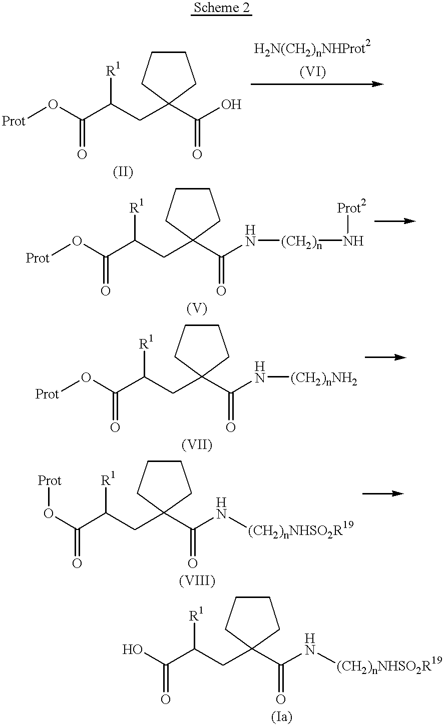

Compounds of formula Ib, i.e. compounds of formula I where n is 0 and Y is

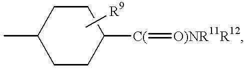

may be prepared according to reaction scheme 3. Compounds of formula II are reacted with compounds of formula IIIa under analogous conditions to acid/amine coupling conditions of Scheme 1 to give compounds of formula IX, where Prot³ is a protecting group which can be selectively removed in the presence of protecting group Prot. A preferred protecting group Prot³ is a base labile ester group. Consequently, treatment of compound of formula IX under basic conditions gives compounds of formula X. Compounds of formula X are reacted with compounds of formula NHR¹¹R¹² under analogous conditions to acid/amine coupling conditions of Scheme 1 to form compounds of formula XI. Deprotection of compounds of formula XI under analogous conditions to the deprotection step in Scheme 1 gives compounds of formula Ib.

Preferred conditions for removal of protecting group Prot³ from IX comprise treatment of IX with sodium hydroxide (1N) in methanol at room temperature for 22 hours.

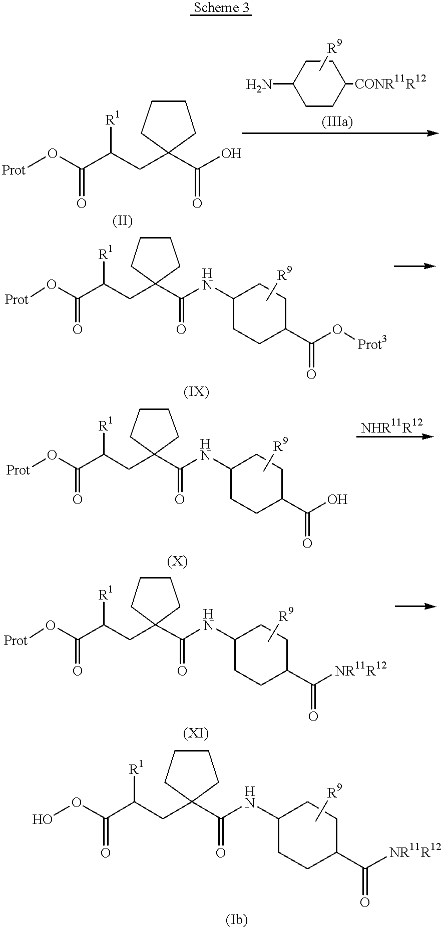

Compounds of formula IIIb, i.e. compounds of general formula III where n is 2 and Y is 2-oxopiperidino, may be prepared according to reaction scheme 4.

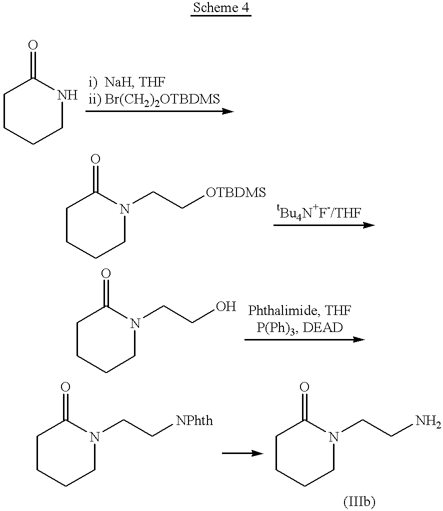

Compounds of formula IIIc where n is 1 or 2, may be prepared according to reaction scheme 5. Compounds of formula XII are protected at the amine moiety with a suitable protecting group Prot⁴ to form compounds of formula XIII. A preferred protecting group is tert-butyloxycarbonyl. Compounds of formula XIII are reacted under typical acid/amine coupling conditions with NHR¹¹R¹² to form compounds of formula XIV, which on deprotection form compounds of formula IIIc.

Typical reaction conditions for introducing the tert-butyloxycarbonyl protecting group comprise treating XII with (tert-butyloxycarbonyl)₂O in dioxan and 2N sodium hydroxide at room temperature for 18 hrs.

Typical acid/amine coupling conditions comprise treating Xil and NHR¹¹R¹² with benzotriazol-1-yloxytris(pyrrolidino)phosphonium hexafluorophosphate (PYBOP), 1-hydroxybenzotrazole hydrate (HOBT), Huünigs base, an amine (eg triethylamine), in dimethylformamide at room temperature for 2 hrs. Alternatively, XIII and NHR¹¹R¹² may be treated with1-(3-dimethylaminopropyl)-3-ethylcarbodiimide hydrochloride, HOBT, N-methyl morpholine (NMM), in dimethylformamide at room temperature for 18 hrs.

Typical reaction conditions for deprotection when Prot⁴ is tert-butyloxycarbonyl comprise reacting XIV with hydrochloric acid or trifluoroacetic acid in dichloromethane at room temperature for 2 to 4 hrs

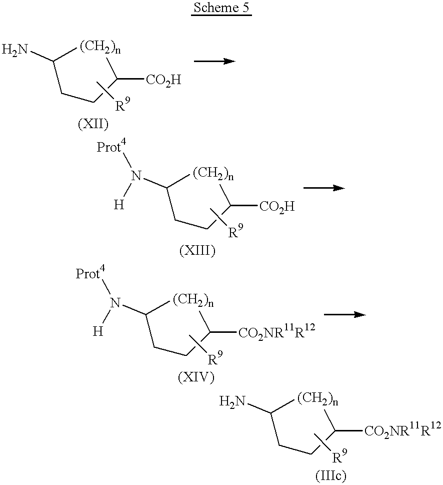

Compounds of formula IIId can be prepared according to reaction scheme 6. The protecting group is preferably tert-butyloxycarbonyl, which is removed under standard conditions, as previously described.

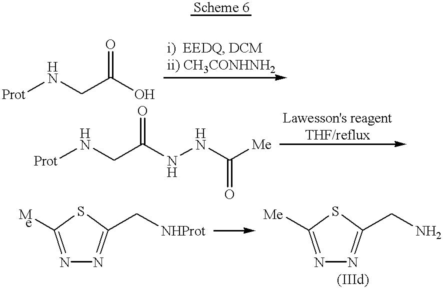

Compounds of formula IIIe are prepared according to reaction scheme 7 using standard acid/amine coupling reactions, as previously described. The protecting group is preferably benzyloxycarbonyl which may be removed under standard conditions, typically palladium on charcoal (5-10%) in ethanol at room temperature and 50 psi for 4 hrs.

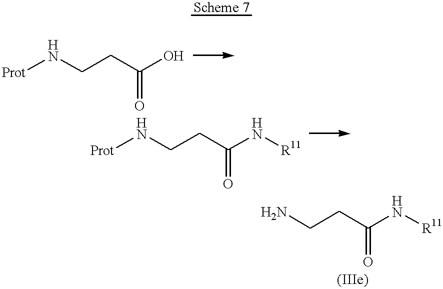

Compounds of formula IIIf may be prepared according to reaction scheme 8.

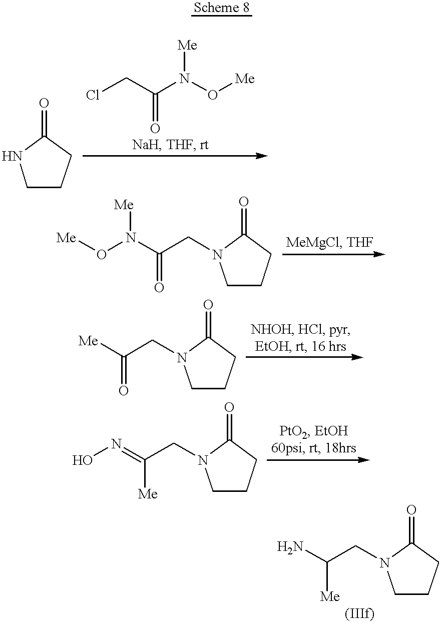

Compounds of formula IIIg may be prepared in two steps according to reaction scheme 9. As a first step, compounds of formula XV are prepared from compounds of formula XVI using standard acid/amine coupling methodology analogous to the acid/amine coupling conditions described for reaction scheme 1. Prot⁵ represents a suitable leaving group, preferably tert-butyloxycarbonyl. The second step comprises removal of Prot⁵. When Prot⁵ is tert-butyloxycarbonyl then preferred reaction conditions comprise treatment with hydrochloric acid in diethyl ether/ethyl acetate at room temperature for 18 hrs.

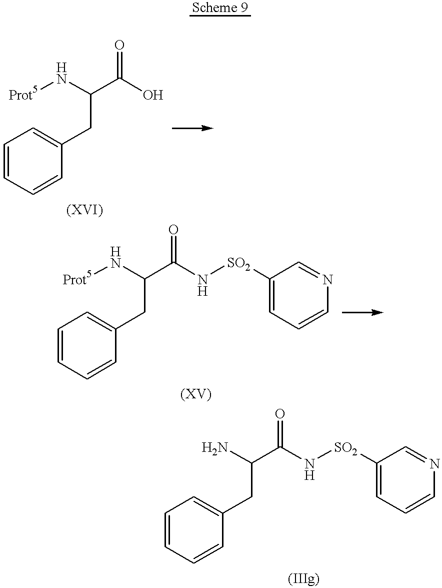

Compounds of formula IIIh may be prepared in three steps according to reaction scheme 10.

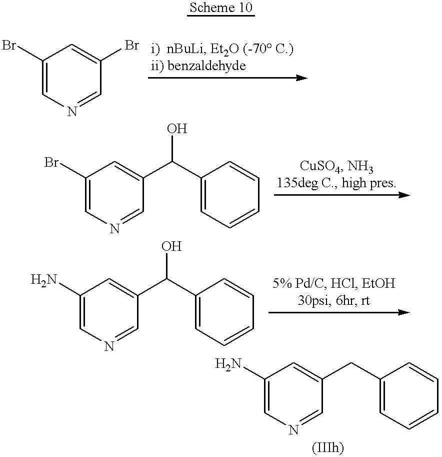

Compounds of formula IIIj may be prepared by reduction of a nitro group according to reaction scheme 11.

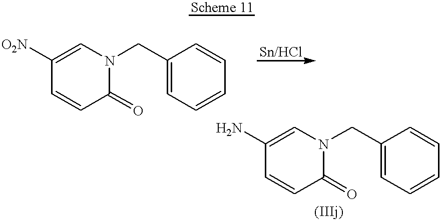

Further methods for preparing compounds of formula III are give in Scheme 12 below, where R^{a} is C₁₋₆alkyl or alkoxy.

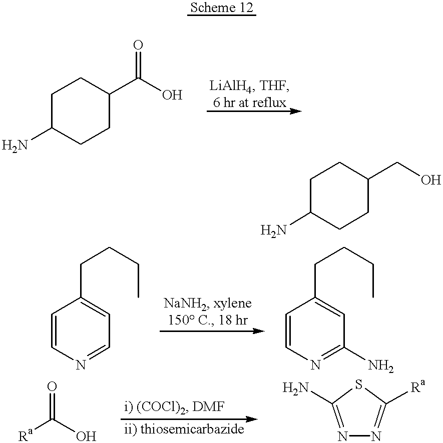

All of the above reactions and the preparations of novel starting materials used in the preceding methods are conventional. Appropriate reagents and reaction conditions for their performance or preparation as well as procedures for isolating the desired products will be well-known to those skilled in the art with reference to literature precedents and the Examples and Preparations hereinbelow.

### PREPARATIVE EXAMPLES

### Example 1

### (2R)-2-[(1-{[(5-ethyl-1,3,4-thiadiazol-2-yl)amino]carbonyl}cyclopentyl) methyl]pentanoic acid and

### Example 2

### (2S)-2-[(1-{[(5-Ethyl-1,3,4-thiadiazol-2-yl)amino]carbonyl}cyclopentyl)-methyl]pentanoic acid

The title product from stage c) below (824 mg) was further purified by HPLC using an AD column and using hexane:iso-propanol:trifluoroacetic acid (85:15:0.2) as elutant to give the title product from Example 1, 400 mg, 99.5% ee, ¹H NMR (CDCl₃, 400 MHz) δ: 0.90 (t, 3H), 1.36 (m, 6H), 1.50-1.80 (m, 9H), 2.19 (m, 1H), 2.30 (m, 1H), 2.44 (m, 1H), 2.60 (m, 1H), 2.98 (q, 2H), 12.10-12.30 (bs, 1H), LRMS: m/z 338 (MH⁻), [α]_{D}=−9.0° (C=0.1, methanol), and the title product from Example 2, 386 mg, 99% ee, ¹H NMR (CDCl₃, 400 MHz) δ: 0.90 (t, 3H), 1.38 (m, 6H), 1.50-1.79 (m, 9H), 2.19 (m, 1H), 2.30 (m, 1H), 2.44 (m, 1H), 2.60 (m, 1H), 2.98 (q, 2H), 12.10-12.27 (bs, 1H); LRMS: m/z 338 (MH⁻); and [α]_{D}=+3.8° (c=0.1, methanol)

### Preparation of Starting Materials

### a) 1-[2-(tert-Butoxycarbonyl)-4-pentyl]-cyclopentane carboxylic acid

A mixture of 1-[2-(tert-butoxycarbonyl)-4-pentenyl]-cyclopentane carboxylic acid (EP 274234) (23 g, 81.5 mmol) and 10% palladium on charcoal (2 g) in dry ethanol (200 ml) was hydrogenated at 30 psi and room temperature for 18 hours. The reaction mixture was filtered through Arbocel®, and the filtrate evaporated under reduced pressure to give a yellow oil. The crude product was purified by column chromatography on silica gel, using ethyl acetate:pentane (40:60) as the eluant, to provide the desired product as a clear oil, 21 g, 91%; ¹H NMR (CDCl₃, 0.86 (t, 3H), 1.22-1.58 (m, 15H), 1.64 (m, 4H), 1.78 (dd, 1H), 2.00-2.18 (m, 3H), 2.24 (m, 1H); LRMS: m/z 283 (M—H)⁻

### b) tert-Butyl 2-[(1-{[(5-ethyl-1,3,4-thiadiazol-2-yl)amino]carbonyl}-cyclopentyl)methyl]pentanoate.

1-(3-Dimethylaminopropyl)-3-ethylcarbodiimide hydrochloride (0.21 mmol), 1-hydroxybenzotriazole hydrate (0.2 mmol), N-methylmorpholine (0.31 mmol) and 2-amino-5-ethyl-1,3,4-thiadiazole (0.22 mmol) were added to a solution of the product from stage a) above (150 mg, 0.53 mmol) in N,N-dimethylformamide (3 ml), and the reaction stirred at 90° C. for 18 hours. The cooled solution was diluted with ethyl acetate (90 ml), washed with water (3×25 ml), and brine (25 ml), then dried (MgSO₄) and evaporated under reduced pressure. The crude product was purified by chromatography on silica gel, using ethyl acetate:pentane (30:70) as the eluant to afford the title compound, 92%; ¹H NMR (CDCl₃, 300 MHz) δ: 0.82 (t, 3H), 1.20-1.80 (m, 22H), 1.84 (m, 1H), 2.20 (m, 4H), 3.04 (q, 2H), 9.10 (bs, 1H); LRMS: m/z 396.2 (MH⁺).

### c) 2-[(1-{[(5-ethyl-1,3,4-thiadiazol-2-yl)amino]carbonyl}cyclopentyl) methyl]pentanoic acid.

Trifluoroacetic acid (5ml) was added to a solution of the title product from stage b) above (0.31 mmol) in dichloromethane (5 ml), and the solution stirred at room temperature for 4 hours. The reaction mixture was concentrated under reduced pressure and the residue azeotroped with toluene and dichloromethane to afford the title compound as a clear oil, 81%, ¹H NMR (CDCl₃, 400 MHz) δ: 0.92 (t, 3H), 1.35 (t, 3H), 1.25-1.80 (m, 11H), 2.20-2.50 (m, 4H), 2.95 (q, 2H), 12.10 (bs, 1H); LRMS: mlz 339.8 (MH⁺); Anal. Found: C, 56.46; H, 7.46; N, 12.36. C₁₆H₂₅N₃O₃S requires C, 56.62; H, 7.44; N, 12.37%.

### Example 3

### (R)-2-{[1-({[2-(Hydroxymethyl)-2,3-dihydro-1H-inden-2-yl]amino}carbonyl)-cyclopentyl]methyl}-4-methoxybutanoic acid and

### Example 4

### (S)-2-{[1-({[2-(Hydroxymethyl)-2,3-dihydro-1H-inden-2-yl]amino}carbonyl)-cyclopentyl]methyl}-4-methoxybutanoic acid

Racemic 2-{[1-({[2-(hydroxymethyl)-2,3-dihydro-1H-inden-2-yl]amino}-carbonyl)-cyclopentyl]methyl}-4-methoxybutanoic acid from Example 5 was purified by HPLC using a Chiralcel OD column (250*20 mm) at ambient temperature using a mixture of 70% hexane containing 0.3% TFA and 0.2% DEA and 30% IPA containing 0.3% TFA and 0.2% DEA at a flow rate of 10 ml/min. Example 3 is the R enantiomer which eluted first after 6 mins (α_{D} 11.00 c1 mg/ml in EtOH). Example 4 is the S enantiomer which eluted second after 7 mins (α_{D} −8.62 c1.07 mg/ml in EtOH).

### Example 5

### 2-{[1-({[2-(Hydroxymethyl)-2,3-dihydro-1H-inden-2-yl]amino}carbonyl)-cyclopentyl]methyl}-4-methoxybutanoic acid

The title product from stage b) below (0.25 mmol) was taken up in a 4M solution of hydrogen chloride in dioxane (10 mls) and stirred for 3 h. Concentrated in vacuo and purified by column chromatography using 5:95 (MeOH:DCM) as eluant to provide the acid as a colourless film; ¹HNMR (CDCl₃, 400 MHz) δ: 1.43-1.76 (m, 7H), 1.80-2.24 (m, 4H), 2.57-2.68 (m, 2H), 3.06 (d, 1H), 3.12 (d, 1H), 3.27 (d, 1H), 3.32 (s, 3H), 3.36-3.48 (m, 2H), 3.80 (d, 1H), 3.87 (d, 1H), 6.04 (s, 1H), 7.16-7.22 (m, 4H).

### Preparation of Starting Materials

### a) 1-[2-(tert-Butoxycarbonyl)-4-methoxybutyl]cyclopentanecarboxylic acid

A solution of 1-(3-tert-butoxy-3-oxopropyl)cyclopentane carboxylic acid (see EP274234, Example 35) in dry tetrahydrofuran (100 ml) was added to a stirred solution of lithium diisopropylamide (130 ml) in a mixture of hexane (52 ml) and tetrahydrofuran (200 ml) at −78° C. under nitrogen. After 1 hour a solution of 2-bromoethyl methyl ether in tetrahydrofuran (100 ml) was added maintaining the temperature at −78° C. The reaction mixture was allowed to warm up to room temperature overnight. The mixture was quenched with water (100 ml) and acidified to pH 1 with 2M hydrochloric acid, and extracted with ethyl acetate (2×150 ml). The combined organic extracts were dried over magnessium sulphate and concentrated in vacuo to give the crude acid which was chromatographed on silica. Elution with increasing proportions of methanol in dichloromethane (neat dichloromethane to 1:50) gave an oil (7.7 g, 25.6 mmol, 52%). Rf 0.3 methanol, dichloromethane 1:20. ¹H NMR (CDCl₃ 400 MHz) δ: 1.4 (s, 9H), 1.4-1.7 (m, 7H), 1.75-1.95 (m, 2H), 2.0-2.15 (m, 3H), 2.3-2.4 (m, 1H), 3.3 (s, 3H), 3.3-3.4 (m, 2H). LRMS: m/z 299 (M—H⁺).

### b) Tert-Butyl-2-{[1-({[2-(hydroxymethyl)-2,3-dihydro-1H-inden-2-yl]amino}carbonyl)-cyclopentyl]methyl}-4-methoxybutanoate

1-(3-Dimethylaminopropyl)-3-ethylcarbodiimide hydrochloride (41 mg, 0.21 mmol), 1-hydroxybenzotriazole hydrate (27 mg, 0.2 mmol), N-methylmorpholine (35 μl, 0.31 mmol) and finally 2-amino-2-(hydroxymethyl)-2,3-dihydro-1H-indene (see WO9110644, Example 8) (0.22 mmol) were added to the product from stage a) above (0.53 mmol) in N,N-dimethylformamide (3 ml), and the reaction stirred at 90° C. for 18 hours. The cooled solution was diluted with ethyl acetate (90 ml), washed with water (3×25 ml), and brine (25 ml), then dried (MgSO₄) and evaporated under reduced pressure. The crude product was purified by chromatography on silica gel, using ethyl acetate:pentane (30:70) as the eluant to afford the title compound, 38 mg, 57%; ¹H NMR (CDCl₃, 400 MHz) δ: 0.88 (t, 3H), 1.29 (m, 3H), 1.41-1.78 (m, 26H), 1.78-1.98 (m, 4H), 2.04 (m, 1H), 2.26 (m, 1H), 3.59 (dd, 1H), 3.70 (dd, 1H), 4.80 (t, 1H), 5.81 (s, 1H); LRMS: m/z 380 (MH⁻).

### Example 6

### 2-[(1-{[(1-Benzyl-6-oxo-1,6-dihydro-3-pvridinyl)amino]carbonyl}cyclopentyl)-methyl]-4-methoxybutanoic acid

A mixture of the product from stage a) below (850 mg, 1.64 mmol), and 5% palladium on charcoal (250 mg) in 40% aqueous ethanol (21 ml), was hydrogenated at 30 psi and room temperature for 30 minutes. The reaction mixture was filtered through Hyflo®, and the filtrate evaporated under reduced pressure. The residual foam was purified by column chromatography on silica gel using dichloromethane:methanol (97:3) as eluant to give the title compound as a white foam, 550 mg, 79%; ¹H NMR (DMSO-d₆, 300 MHz) δ: 1.24-2.17 (m, 12H), 2.18-2.31 (m, 1H), 3.07 (s, 3H), 3.21 (t, 2H), 5.08 (s, 2H), 6.63 (d, 1H), 7.23-7.41 (m, 5H), 7.72 (d, 1H), 8.24 (s, 1H); Anal. Found: C, 67.46; H, 7.18; N, 6.24. C₂₄H₃₀N₂O₅ requires C, 67.58; H, 7.09; N, 6.57%.

### Preparation of Starting Materials

### a) Benzyl 2-[(1-{[(1-benzyl-6-oxo-1,6-dihydro-3-pyridinyl)amino]carbonyl}cyclopentyl)-methyl]-4-methoxybutanoate

Oxalyl chloride (0.26 ml, 3.0 mmol) was added to an ice-cooled solution of 1-{2-[(benzyloxy)carbonyl]-4-methoxybutyl}cyclopentanecarboxylic acid (EP 274234, Example 15) (1.0 g, 3.0 mmol) and N,N-dimethylformamide (2 drops) in dichloromethane (20 ml), and the reaction stirred at room temperature for 2 hours. The solution was concentrated under reduced pressure and the residue azeotroped with dichloromethane (3×10 ml). The product was dissolved in dichloromethane (20 ml), then cooled in an ice-bath. The title product from stage b) below (600 mg, 3 mmol) and N-methylmorpholine (0.6 ml, 5.45 mmol) were added and the reaction stirred at room temperature for 18 hours. The reaction mixture was concentrated under reduced pressure, and partitioned between water and ether. The organic layer was washed with hydrochloric acid (2N), sodium bicarbonate solution, then water, dried (MgSO₄) and evaporated under reduced pressure. The residual green solid was purified by medium pressure column chromatography on silica gel using ethyl acetate:hexane (90:10) as eluant to afford the title compound, 880 mg, 57%; ¹H NMR (CDCl₃, 300 MHz) δ: 1.37-2.28 (m, 12H), 2.46-2.64 (m, 1H), 3.20 (s, 3H), 3.31 (m, 2H), 4.97 (dd, 2H), 5.08 (dd, 2H), 6.57 (d, 1H), 7.12 (m, 1H), 7.18-7.48 (m,1 OH), 8.08 (d,1H).

### b) 5-Amino-1-benzyl-2(1H)-pvridinone

A mixture of 1-benzyl-5-nitro-1H-pyridin-2-one (Justus Liebigs Ann. Chem. 484; 1930; 52) (1.0 g, 4.35 mmol), and granulated tin (3.5 g, 29.5 mmol) in concentrated hydrochloric acid (14 ml) was heated at 90° C. for 1.5 hours. The cooled solution was diluted with water, neutralised using sodium carbonate solution, and extracted with ethyl acetate (250 ml in total). The combined organic extracts were filtered, dried (MgSO₄), and evaporated under reduced pressure to give the title compound as a pale green solid, (turned blue with time), 440 mg, 51%; ¹H NMR (CDCl₃, 250 MHz) δ: 4.12-4.47 (bs, 2H), 5.00 (s, 2H), 6.31 (d, 1H), 6.86 (s, 1H), 7.07 (m, 1H), 7.14-7.42 (m, 5H).

### Example 7

### 2-{[1-({[3-(2-Oxo-1-prrolidinyl)propyl]amino}carbonylcyclopentyl]-methyl}-4-phenylbutanoic acid.

A mixture of the starting material (780 mg, 1.55 mmol) and 10% palladium on charcoal (100 mg) in ethanol:water (90:10 by volume; 30 ml) was hydrogenated at room temperature under 60 psi H₂ pressure for 1.5 hours. The catalyst was filtered off, and the filtrate evaporated under reduced pressure to provide the title compound as a white foam, 473 mg, 74%; ¹H NMR (CDCl₃, 300 MHz) δ: 1.26-1.77 (m, 10H), 1.78-2.46 (m, 11H), 2.49-2.70 (m, 2H), 2.95-3.36 (m, 4H), 6.92-7.38 (m, 5H); Anal. Found: C, 64.05; H, 7.73; N, 6.22. C₂₄H₃₄N₂O₄;0.75H₂O requires C, 65.88; H, 7.83; N, 6.40%.

### Preparation of Starting Materials

### Benzyl 2-{[1-({[3-(2-Oxo-1-pyrrolidinyl)propyl]amino}carbonylcyclopentyl]-methyl}-4-phenylbutanoate

1-(3-Dimethylaminopropyl)-3-ethylcarbodiimide hydrochloride (1.06 g, 5.53 mmol), 1-hydroxybenzotriazole hydrate (0.60 g, 4.44 mmol) and 4-methylmorpholine (0.56 g, 5.54 mmol) were added sequentially to a cooled solution of 1-{2-[(benzyloxy)carbonyl]-4-phenylbutyl}cyclopentanecarboxylic acid (EP 274234, Example 17) (1.5 g, 3.94 mmol) in dry dichloromethane (15 ml) at room temperature, followed by N-(3-aminopropyl)-2-pyrrolidinone (Ex. Aldrich Chemical Co.) (0.56 g, 3.94 mmol), and the reaction stirred at room temperature for 18 hours. The mixture was washed with water, 2N hydrochloric acid, saturated aqueous sodium bicarbonate solution, and then dried (MgSO₄) and evaporated under reduced pressure. The residual yellow oil was purified by column chromatography on silica gel using ethyl acetate:pentane (50:50) as the eluant to provide the title compound as a clear gum, 800 mg, 40%; ¹H NMR (CDCl₃, 300 MHz) δ: 1.37-2.20 (m, 16H), 2.34-2.58 (m, 5H), 2.92-3.46 (m, 6H), 5.07 (d, 1H), 5.18 (d, 1H), 6.98-7.47 (m, 10H).

### Example 8

### (R)-2-{[1-({[2-(Hydroxymethyl)-2,3-dihydro-1H-inden-2-yl]amino}carbonyl)-cyclopentyl]-methyl}-4-phenylbutanoic acid and

### Example 9

### (S)-2-{[1-({[2-(Hydroxymethyl)-2,3-dihydro-1H-inden-2-yl]amino}carbonyl)-cyclopentyl]methyl}-4-phenylbutanoic acid

2-{[1-({[2-(Hydroxymethyl)-2,3-dihydro-1H-inden-2-yl]amino}carbonyl)-cyclopentyl]methyl}-4-phenylbutanoic acid (WO 9110644, Example 9) was purified by standard HPLC procedures using an AD column and hexane:isopropanol: trifluoroacetic acid (70:30:0.2) as eluant, to give the title compound of Example 8, 99.5% ee; [α]_{D}=+9.1° (C=1.76 in ethanol); and the title compound of Example 9, 99.5% ee; [α]_{D}=−10.5° (c=2.2 in ethanol).

### Example 10

### 2-[(1-{[(5-Methyl-1,3,4-thiadiazol-2-yl)amino]carbonyl}cyclopentyl)methyl]-4-phenylbutanoic acid

A mixture of the starting material (187 mg, 0.39 mmol) and 10% palladium on charcoal (80 mg) in ethanol (20 ml) was hydrogenated at 60 psi for 18 hours. Tlc analysis showed starting material remaining, so additional 10% palladium on charcoal (100 mg) was added, and the reaction continued for a further 5 hours. Tlc analysis again showed starting material remaining, so additional catalyst (100 mg) was added, and hydrogenation continued for 18 hours. The mixture was filtered through Arbocel®, and the filtrate concentrated under reduced pressure, and azeotroped with dichloromethane. The crude product was purified by chromatography on silica gel using a Biotage® column, and dichloromethane:methanol (95:5) as eluant to afford the title compound as a clear oil, 80 mg, 53%; ¹H NMR (CDCl₃, 300 MHz) δ: 1.51-1.89 (m, 9H), 2.03 (m, 1H), 2.20 (m, 1H), 2.40 (m, 2H), 2.60 (m, 5H), 7.15-7.30 (m, 5H); LRMS: m/z 387.8 (MH⁺).

### Preparation of Starting Materials

### Benzyl 2-[(1-{[(5-methyl-1,3,4-thiadiazol-2-yl)amino]carbonyl}cyclopentyl)methyl]-4-phenylbutanoate

1-(3-Dimethylaminopropyl)-3-ethylcarbodiimide hydrochloride (122 mg, 0.64 mmol), 1-hydroxybenzotriazole hydrate (86 mg, 0.64 mmol) and 4-methylmorpholine (173 μl, 1.59 mmol) were added sequentially to a cooled solution of 1-{2-[(benzyloxy)carbonyl]-4-phenylbutyl}cyclopentane-carboxylic acid (EP 274234, Example 17) (202 mg, 0.53 mmol) in N,N-dimethylformamide (5 ml) at room temperature, followed by 2-amino-5-methyl-1,3,4-thiadiazole (ex Lancaster) (1.06 mmol), and the reaction stirred at 90° C. for 18 hours. The cooled solution was concentrated under reduced pressure and the residue partitioned between water (20 ml) and ethyl acetate (100 ml). The layers were separated, the organic phase washed with water (3×30 ml), brine (25 ml) dried (MgSO₄), and evaporated under reduced pressure to give a clear oil. The crude product was purified by column chromatography on silica gel using dichloromethane:methanol (98:2) as eluant to afford the title compound, 74%; ¹H NMR (CDCl₃, 400 MHz) δ: 1.58-1.76 (m, 7H), 1.83-1.98 (m, 3H), 2.03 (m, 1H), 2.20 (m, 1H), 2.35 (m, 1H), 2.44 (m, 3H), 2.65 (s, 3H), 5.02 (dd, 2H), 7.00 (d, 2H), 7.15 (m, 1H), 7.19 (m, 2H), 7.35 (m, 5H); LRMS: m/z 478.7 (MH⁺).

### Example 11

### cis-3-(2-Methoxyethoxy)-2-[(1-{[(4-{[(phenylsulfonyl)amino]carbonyl}-cyclohexyl)amino]carbonyl}cyclopentyl)methyl]propanoic acid

A solution of the starting material from stage b) below (446 mg, 0.75 mmol) in dichloromethane (5 ml) and trifluoroacetic acid (5 ml) was stirred at room temperature for 18 hours. The reaction mixture was concentrated under reduced pressure, and the residue azeotroped with dichloromethane, then toluene, and finally ether, to afford the title compound as a white foam, 385 mg, 95%; ¹H NMR (CDCl₃, 400 MHz) δ: 1.48-2.17 (m, 18H), 2.40 (s, 1H), 2.66 (s, 1H), 3.37 (s, 3H), 3.50-3.70 (m, 6H), 3.94 (s, 1H), 6.10 (d, 1H), 6.59 (s, 1H), 7.55 (t, 2H), 7.61 (m, 1H), 8.02 (d, 2H), 9.11 (s, 1H); Anal. Found: C, 54.88; H, 6.90; N, 5.04. C₂₆H₃₈N₂O₈S;1.7H₂O requires C, 57.97; H, 7.11; N, 5.20%.

### Preparation of Starting Materials

### a) 4-{[(1-{3-tert-Butoxy-2-[(2-methoxyethoxy)methyl]-3-oxopropyl}cyclopentyl)-carbonyl]amino}cyclohexanecarboxylic acid

A mixture of benzyl 4-{[(1-{3-tert-butoxy-2-[(2-methoxyethoxy)methyl]-3-oxopropyl}cyclopentyl)carbonyl]amino}cyclohexanecarboxylate (EP 274234, Example 96), and 10% palladium on charcoal (250 mg) in water (10 ml) and ethanol (50 ml) was hydrogenated at 50 psi and room temperature for 18 hours. The reaction mixture was filtered through Solkafloc®, the filtrate concentrated under reduced pressure and the residue azeotroped with toluene (3×) and then dichloromethane (3×), to give the title compound, 2.0 g, 96%; ¹H NMR (CDCl₃, 300 MHz) δ: 1.48 (s, 9H), 1.53-1.84 (m, 14H), 1.94-2.10 (m, 5H), 2.60 (m, 2H), 3.40 (s, 3H), 3.41-3.63 (m, 5H), 3.96 (m, 1H), 5.90 (bd, 1H).

### b) cis-tert-Butyl 3-(2-methoxyethoxy)-2-[(1-{[(4-{[(phenylsulfonyl)amino]-carbonyl}cyclohexyl)amino]carbonyl}cyclopentyl)methyl]propanoate

N,N′-Dicyclohexylcarbodiimide (199 mg, 0.97 mmol), 4-dimethylaminopyridine (118 mg, 0.97 mmol) and benzenesulphonamide (152 mg, 0.97 mmol) were added to an ice-cooled solution of the product from stage a) above (400 mg, 0.878 mmol) in dichloromethane (12 ml) and N,N-dimethylformamide (0.5 ml), and the reaction stirred at room temperature for 20 hours. The mixture was concentrated under reduced pressure and the residue suspended in cold ethyl acetate. The resulting insoluble material was filtered off, the filtrate washed with hydrochloric acid (1N), and water, then dried (MgSO₄) and evaporated under reduced pressure. The crude product was purified by column chromatography on silica gel using an elution gradient of dichloromethane:methanol (95:5 to 90:10) to afford the title compound as a white foam, 480 mg, 92%; ¹H NMR (CDCl₃, 400 MHz) δ: 1.44 (s, 9H), 1.63 (m, 13H), 1.80 (m, 2H), 1.88 (m,1H), 1.98 (m, 2H), 2.36 (m,1H), 2.57 (m, 1H), 3.38 (s, 3H), 3.40 (m, 1H), 3.51 (t, 2H), 3.58 (m, 3H), 3.95 (m, 1H), 5.92 (d, 1H), 7.56 (m, 2H), 7.62 (m, 1H), 8.05 (d, 2H), 8.75 (bs, 1H); LRMS: m/z 618 (MNa⁺).

### Example 12

### (R)-2-{[1-({[2-(Hydroxymethyl)-2,3-dihydro-1H-inden-2-yl]amino}carbonyl)-cyclopentyl]methyl}pentanoic acid and

### Example 13

### (S)-2-{[1-({[2-(Hydroxymethyl)-2,3-dihydro-1H-inden-2-yl]amino}carbonyl)-cyclopentyl]methyl}pentanoic acid

2-{[1-({[2-(Hydroxymethyl)-2,3-dihydro-1H-inden-2-yl]amino}carbonyl)-cyclopentyl]methyl}pentanoic acid (WO 9110644, Example 8) was further purified by HPLC using an AD column and hexane:isopropanol:trifluoroacetic acid (90:10:0.1) as eluant, to give the title compound of Example 31, 99% ee, [α]_{D}=+10.4° (c=0.067, ethanol) and the title compound of Example 32, 99% ee, [α]_{D}=−10.9° (c=0.046, ethanol).

Details on a suitable assay system for identifying and/or studying a NEPi (I:NEP) are presented in the hereinafter in the section entitled NEP Assay.

Further examples of NEP inhibitors are disclosed and discussed in the following review articles:

Pathol. Biol., 46(3), 1998,191.

Current Pharm. Design, 2(5), 1996, 443.

Biochem. Soc. Trans., 21(3), 1993, 678.

Handbook Exp. Pharmacol., 104/1, 1993, 547.

TiPS, 11, 1990, 245.

Pharmacol. Rev., 45(1), 1993, 87.

Curr. Opin. Inves. Drugs, 2(11), 1993, 1175.

Antihypertens. Drugs, (1997), 113.

Chemtracts, (1997), 10(11), 804.

Zinc Metalloproteases Health Dis. (1996), 105.

Cardiovasc. Drug Rev., (1996), 14(2), 166.

Gen. Pharmacol., (1996), 27(4), 581.

Cardiovasc. Drug Rev., (1994), 12(4), 271.

Clin. Exp. Pharmacol. Physiol., (1995), 22(1), 63.

Cardiovasc. Drug Rev., (1991), 9(3), 285.

Exp. Opin. Ther. Patents (1996), 6(11), 1147.

Yet, further examples of NEPi's are disclosed in the following documents:

EP-509442A

U.S. Pat. No. 192,435

U.S. Pat. No. 4,929,641

EP-599444B

U.S. Pat. No. 884,664

EP-544620A

U.S. Pat. No. 798,684

J. Med. Chem. 1993, 3821.

Circulation 1993, 88(4), 1.

EP-136883

JP-85136554

U.S. Pat. No. 4,722,810

Curr. Pharm. Design, 1996, 2, 443.

EP-640594

J. Med. Chem. 1993, 36(1), 87.

EP-738711-A

JP-270957

CAS #115406-23-0

DE-19510566

DE-19638020

EP-830863

JP-98101565

EP-733642

WO9614293

JP-08245609

JP-96245609

WO9415908

JP05092948

WO-9309101

WO-9109840

EP-519738

EP-690070

J. Med. Chem. (1993), 36, 2420.

JP-95157459

Bioorg. Med. Chem. Letts., 1996, 6(1), 65.

Further I:NEPs are disclosed in the following documents:

EP-A-0274234

JP-88165353

Biochem.Biophys.Res. Comm.,1989, 164, 58

EP-629627-A

U.S. Pat. No. 77,978

Perspect. Med. Chem. (1993), 45.

EP-358398-B

Further examples of I:NEPs are selected from the following structures:

| | | Mode of Action |
|---|---|---|
| Compound | Structure | References |
| FXII | 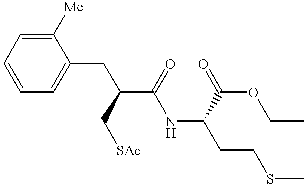 | I:NEP EP-509442A US-192435 US-4929641 |
| FXIII | 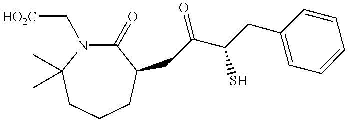 | I:NEP (also an ACE inhibitor) EP-599444B US-884664 |
| FXIV | 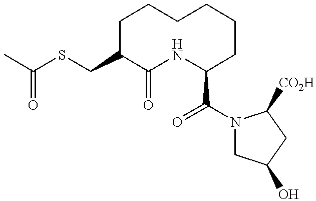 | I:NEP EP-544620A US-798684 J. Med. Chem. 1993, 3821. |
| FXV | 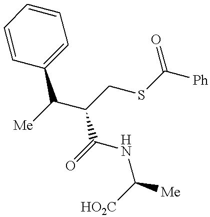 | I:NEP (also an ACE inhibitor) Mixanpril Circulation 1993, 88(4), 1. |
| FXVI | 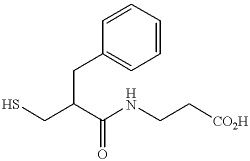 | I:NEP EP-136883 JP-85136554 US-4722810 |
| FXVII | 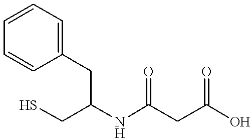 | I:NEP Retrothiorphan Curr. Pharm. Design, 1996, 2, 443. |
| FXVIII | 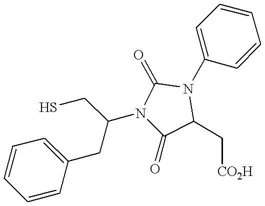 | I:NEP (also an ACE inhibitor) EP-640594 |
| FXIX | 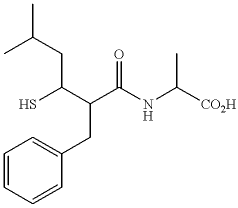 | I:NEP J. Med. Chem. 1993, 36(1), 87. |
| FXX | 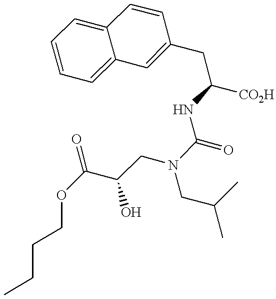 | I:NEP (also an ACE inhibitor) EP-738711-A JP-270957 |
| FXXI | 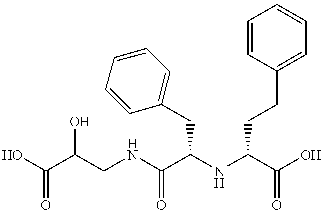 | I:NEP CAS #115406-23-0 |
| FXXII | 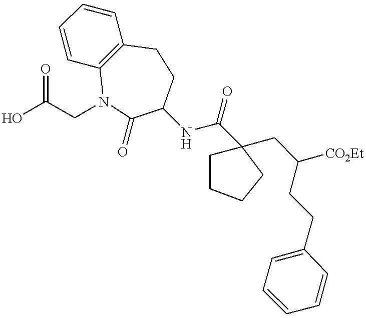 | I:NEP (also an ECE inhibitor) DE-19510566 DE-19638020 EP-830863 JP-98101565 |
| FXXIII | 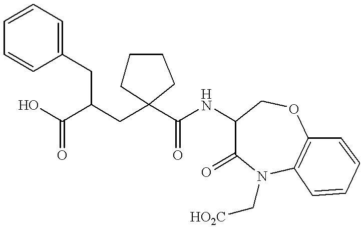 | I:NEP (also an ECE inhibitor) EP-733642 |
| FXXIV | 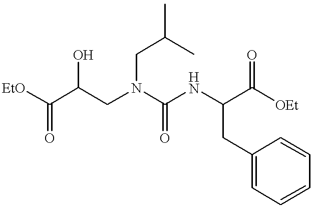 | I:NEP WO96/14293 |
| FXXV | 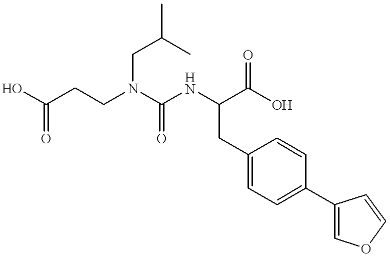 | I:NEP JP-08245609 JP-96245609 |
| FXXVI | 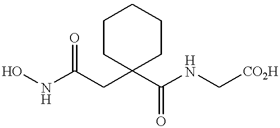 | I:NEP WO9415908 |
| FXXVII | 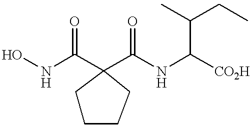 | I:NEP JP05092948 |
| FXXVIII | 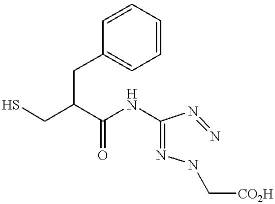 | I:NEP WO-9309101 |
| FXXIX | 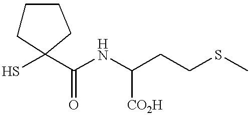 | I:NEP WO-9109840 |
| FXXXI | 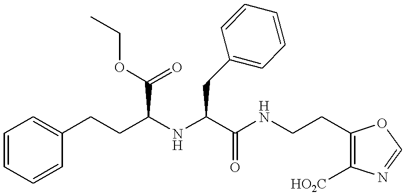 | I:NEP EP-519738 EP-690070 |
| FXXXII | 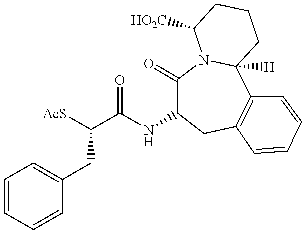 | I:NEP (also an ACE inhibitor) J.Med. Chem. (1993), 36,2420. |
| FXXXIII | 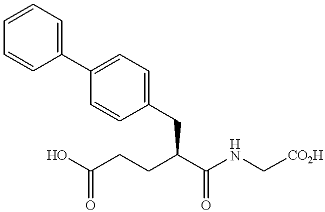 | I:NEP JP-95157459 Bioorg. Med. Chem. Letts., 1996, 6(1), 65. |

Additional I:NEPs(together with references of how to make them—incorporated herein by reference) are selected from the following structures:

| | | Mode of Action |
|---|---|---|
| Compound | Structure | References |
| FV | 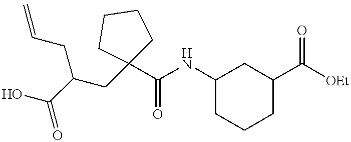 | I:NEP EP-A-0274234 (Example 300) |
| FVI | 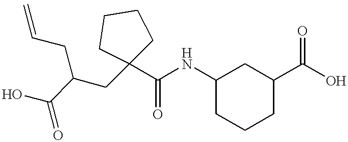 | I:NEP EP-A-0274234 (Example 379) |
| FVII | 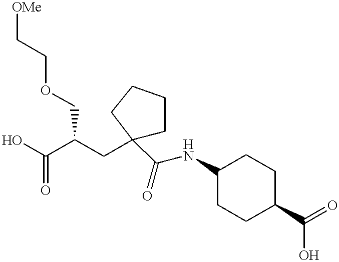 | I:NEP Candoxatrilat EP-274234 JP-88165353 Biochem. Biophys. Res. Comm., 1989, 164, 58 |
| FVIII | 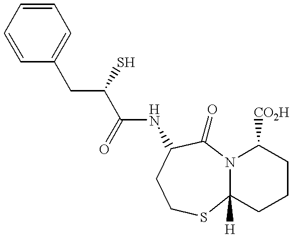 | I:NEP Imapatrilat (also an inhibitor of ACE) EP-0629627-A US-77978 |
| FIX | 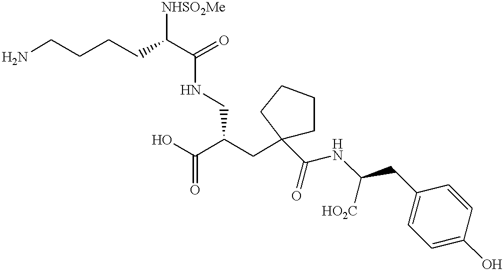 | I:NEP Sampatrilat (also an inhibitor of ACE) Perspect. Med. Chem. (1993), 45. EP-0358398-B |
| FX | 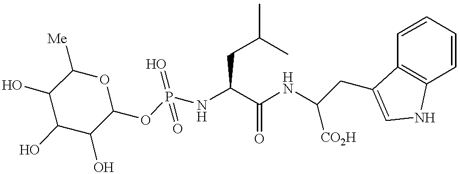 | I:NEP Phosphoramidon (which is commercially available) |
| FXI | 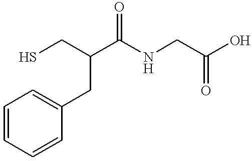 | I:NEP Thiorphan (which is commercially available) |

The suitability of any particular NEP inhibitor (or PDE5i or other additional active compound used in a combination of the invention) can be readily determined by evaluation of its potency and selectivity using literature methods followed by evaluation of its toxicity, absorption, metabolism, pharmacokinetics, etc in accordance with standard pharmaceutical practice.

### Combinations

The NEPi, and where present PDE5i compounds, useful for the treatment of MED according to the present invention, may also be used in combination with one or more additional pharmaceutically active agents. The additional pharmaceutically active agent(s) as defined hereinbefore, if present, may be referred to as an “additional agent”. One or more of such additional agents may be one or more of: PDEi, another NEPi, or an NPYi. Combinations of agents are discussed in more detail below. Thus although a particularly preferred aspect of the invention is NEPi in combination with a PDE5i, other combinations of NEPi and active agents (other than PDE5 are also within the scope of the inventions). Reference herein to invention also includes combination of NEPi with other additional (active) agents.

General references herein to agents may be applicable to additional agents as well as to NEPi or PDE5i compounds.

In accordance with the use of NEPi compounds for the treatment of MED according to the as discussed hereinbefore, the NEPi acts on a target, preferably specifically on that target. For example where a combination of a NEPi and a PDE5i are present the targets are the NEP and PDE5 enzymes. This target is sometimes referred to as the “target of the present invention”. However, the additional agents of the present invention may act on one or more other targets. These other targets may be referred to as an “additional target”. Likewise, if an additional agent is used, then that additional agent can target the same target of the present invention and/or an additional target (which need not be the same additional target that is acted on by the agent of the present invention). Targets are described herein. It is to be understood that general references herein to targets may be applicable to the additional targets as well as to the target of the present invention.

The present invention additionally comprises the combination of a NEPi for the treatment of male sexual dysfunction as outlined herein (more particularly male erectile dysfunction) with one or more of the following additional active agents.

Thus a further aspect of the invention provides a pharmaceutical combination (for simultaneous, separate or sequential administration) of a NEPi according to the invention and:

1) one or more naturally occurring or synthetic prostaglandins or esters thereof. Suitable prostaglandins for use herein include compounds such as alprostadil, prostaglandin E₁, prostaglandin E₀, 13, 14-dihydroprosta glandin E₁, prostaglandin E₂, eprostinol, natural synthetic and semi-synthetic prostaglandins and derivatives thereof including those described in WO-00033825 and/or U.S. Pat. No. 6,037,346 issued on Mar. 14, 2000 all incorporated herein by reference, PGE₀, PGE₁, PGA₁, PGB₁, PGF₁α, 19-hydroxy PGA₁, 19-hydroxy-PGB₁, PGE₂, PGB₂, 19-hydroxy-PGA₂, 19-hydroxy-PGB₂, PGE₃α, carboprost tromethamine dinoprost, tromethamine, dinoprostone, lipo prost, gemeprost, metenoprost, suiprostune, tiaprost and moxisylate; and/or

2) one or more α-adrenergic receptor antagonist compounds also known as α-adrenoceptors or α-receptors or α-blockers. Suitable compounds for use herein include: the α-adrenergic receptor blockers as described in PCT application WO99/30697 published on Jun. 14, 1998, the disclosures of which relating to α-adrenergic receptors are incorporated herein by reference and include, selective αₐ-adrenoceptor or α₂-adrenoceptor blockers and non-selective adrenoceptor blockers, suitable α₁-adrenoceptor blockers include: phentolamine, phentolamine mesylate, trazodone, alfuzosin, indoramin, naftopidil, tamsulosin, dapiprazole, phenoxybenzamine, idazoxan, efaraxan, yohimbine, rauwolfa alkaloids, Recordati 15/2739, SNAP 1069, SNAP 5089, RS17053, SL 89.0591, doxazosin, terazosin, abanoquil and prazosin; α₂-blocker blockers from U.S. Pat. No. 6,037,346 [Mar. 14, 2000] dibenarnine, tolazoline, trimazosin and dibenarnine; α-adrenergic receptors as described in U.S. Pat. Nos. 4,188,390; 4,026,894; 3,511,836; 4,315,007; 3,527,761; 3,997,666; 2,503,059; 4,703,063; 3,381,009; 4,252,721 and 2,599,000 each of which is incorporated herein by reference; α₂-Adrenoceptor blockers include: clonidine, papaverine, papaverine hydrochloride, optionally in the presence of a cariotonic agent such as pirxamine; and/or

3) one or more NO-donor (NO-agonist) compounds. Suitable NO-donor compounds for use herein include organic nitrates, such as mono- di or tri-nitrates or organic nitrate esters including glyceryl brinitrate (also known as nitroglycerin), isosorbide 5-mononitrate, isosorbide dinitrate, pentaerythritol tetranitrate, erythrityl tetranitrate, sodium nitroprusside (SNP), 3-morpholinosydnonimine molsidomine, S-nitroso-N-acetyl penicilliamine (SNAP) S-nitroso-N-glutathione (SNO-GLU), N-hydroxy-L-arginine, amylnitrate, linsidomine, linsidomine chlorohydrate, (SIN-1) S-nitroso-N-cysteine, diazenium diolates,(NONOates), 1,5-pentanedinitrate, L-arginene, ginseng, zizphi fructus, molsidomine, Re - 2047, nitrosylated maxisylyte derivatives such as NMI-678-11 and NMI-937 as described in published PCT application WO 0012075; and/or

4) one or more potassium channel openers or modulators. Suitable potassium channel openers/modulators for use herein include nicorandil, cromokalim, levcromakalim, lemakalim, pinacidil, cliazoxide, minoxidil, charybdotoxin, glyburide, 4-amini pyridine, BaCl₂; and/or

5) one or more dopaminergic agents, preferably apomorphine or a selective D2, D3 or D2/D₃agonist such as, pramipexole and ropirinol (as claimed in WO-0023056),L-Dopa or carbidopa, PNU95666 (as claimed in WO-0040226); and/or

6) one or more vasodilator agents. Suitable vasodilator agents for use herein include nimodepine, pinacidil, cyclandelate, isoxsuprine, chloroprumazine, halo peridol, Rec 15/2739, trazodone, and/or

7) one or more thromboxane A2 agonists; and/or

8) one or more ergot alkoloids; Suitable ergot alkaloids are described in U.S. Pat. No. 6,037,346 issued on Mar. 14, 2000 and include acetergamine, brazergoline, bromerguride, cianergoline, delorgotrile, disulergine, ergonovine maleate, ergotamine tartrate, etisulergine, lergotrile, lysergide, mesulergine, metergoline, metergotamine, nicergoline, pergolide, propisergide, proterguride, terguride; and/or

9) one or more compounds which modulate the action of natruretic factors in particular atrial naturetic factor (also known as atrial naturetic peptide), B type and C type naturetic factors; and/or

10) one or more angiotensin receptor antagonists such as losartan; and/or

11) one or more substrates for NO-synthase, such as L-arginine; and/or

12) one or more calcium channel blockers such as amlodipine; and/or

13) one or more antagonists of endothelin receptors and inhibitors or endothelin-converting enzyme; and/or

14) one or more cholesterol lowering agents such as statins (e.g. atorvastatin/Lipitor-trade mark) and fibrates; and/or

15) one or more antiplatelet and antithrombotic agents, e.g. tPA, uPA, warfarin, hirudin and other thrombin inhibitors, heparin, thromboplastin activating factor inhibitors; and/or

16) one or more insulin sensitising agents such as rezulin and hypoglycaemic agents such as glipizide; and/or

17) one or more acetylcholinesterase inhibitors such as donezipil; and/or

18) one or more estrogen receptor modulators and/or estrogen agonists and/or estrogen antagonists, preferably raloxifene or lasofoxifene, (−)-cis-6-phenyl-5-[4-(2-pyrrolidin-1-yl-ethoxy)-phenyl]-5,6,7,8-tetrahydronaphthalene-2-ol and pharmaceutically acceptable salts thereof (compound A below) the preparation of which is detailed in WO 96/21656.

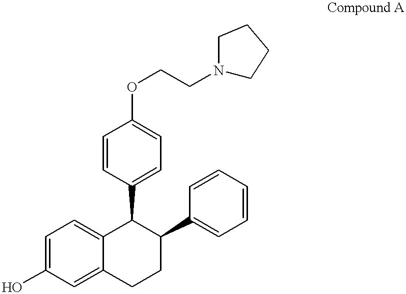

23) one or more of a PDE inhibitor, more particularly a PDE 2, 4, 5, 7 or 8 inhibitor, preferably PDE2 or PDE5 inhibitor and most preferably a PDE5 inhibitor (see hereinafter), said inhibitors preferably having an IC50 against the respective enzyme of less than 100 nM: and/or

24) one or more of an NPY (neuropeptide Y) inhibitor, more particularly NPY1 or NPY5 inhibitor, preferably NPY1 inhibitor, preferably said NPY inhibitors (including NPY Y1 and NPY Y5) having an IC50 of less than 100 nM, more preferably less than 50 nM; and/or

25) one or more of vasoactive intestinal protein (VIP), VIP mimetic, more particularly mediated by one or more of the VIP receptor subtypes VPAC1,VPAC or PACAP (pituitary adenylate cyclase activating peptide), one or more of a VIP receptor agonist or a VIP analogue (eg Ro-125-1553) or a VIP fragment, one or more of a α-adrenoceptor antagonist with VIP combination (eg lnvicorp, Aviptadil); and/or

26) one or more of a melanocortin receptor agonist or modulator or melanocortin ehancer, such as melanotan II, PT-14, PT-141 or compounds claimed in WO-09964002, WO-00074679, WO-09955679, WO-00105401, WO-00058361, WO-00114879, WO-00113112, WO-09954358 and/or

27) one or more of a serotonin receptor agonist, antagonist or modulator, more particularly agonists, antagonists or modulators for 5HT1A (including VML 670), 5HT2A, 5HT2C, 5HT3 and/or 5HT6 receptors, including those described in WO-09902159, WO-00002550 and/or WO-00028993; and/or

28) one or more of a testosterone replacement agent (inc dehydroandrostendione), testosternone (Tostrelle), dihydrotestosterone or a testosterone implant; and/or

29)one or more of estrogen, estrogen and medroxyprogesterone or medroxyprogesterone acetate (MPA) (i.e. as a combination), or estrogen and methyl testosterone hormone replacement therapy agent (e.g. HRT especially Premarin, Cenestin, Oestrofeminal, Equin, Estrace, Estrofem, Elleste Solo, Estring, Eastraderm TTS, Eastraderm Matrix, Dermestril, Premphase, Preempro, Prempak, Premique, Estratest, Estratest HS, Tibolone); and/or

30) one or more of a modulator of transporters for noradrenaline, dopamine and/or serotonin, such as bupropion, GW-320659

31) one or more of a purinergic receptor agonist and/or modulator; and/or

32) one or more of a neurokinin (NK) receptor antagonist, including those described in WO-09964008; and/or

33) one or more of an opioid receptor agonist, antagonist or modulator, preferably agonists for the ORL-1 receptor and/or;

34) one or more of an agonist or modulator for oxytocin/vasopressin receptors, preferably a selective oxytocin agonist or modulator and/or;

35) one or more modulators of cannabinoid receptors.

### Additional Agent PDE5 inhibitor (I:PDE5)

### PDE5 Inhibitors

Suitable PDE5i's for use in the pharmaceutical combinatiions according to the present invention are the cGMP PDE5i's hereinafter detailed. Particularly preferred for use herein are potent and selective cGMP PDE5i's.

Suitable cGMP PDE5 inhibitors for the use according to the present invention include:

the pyrazolo [4,3-d]pyrimidin-7-ones disclosed in EP-A-0463756; the pyrazolo [4,3-d]pyrimidin-7-ones disclosed in EP-A-0526004; the pyrazolo [4,3-d]pyrimidin-7-ones disclosed in published international patent application WO 93/06104; the isomeric pyrazolo [3,4-d]pyrimidin-4-ones disclosed in published international patent application WO 93/07149; the quinazolin-4-ones disclosed in published international patent application WO 93/12095; the pyrido [3,2-d]pyrimidin-4-ones disclosed in published international patent application WO 94/05661; the purin-6-ones disclosed in published international patent application WO 94/00453; the pyrazolo [4,3-d]pyrimidin-7-ones disclosed in published international patent application WO 98/49166; the pyrazolo [4,3-d]pyrimidin-7-ones disclosed in published international patent application WO 99/54333; the pyrazolo [4,3-d]pyrimidin-4-ones disclosed in EP-A-0995751; the pyrazolo [4,3-d]pyrimidin-7-ones disclosed in published international patent application WO 00/24745; the pyrazolo [4,3-d]pyrimidin-4-ones disclosed in EP-A-0995750; the compounds disclosed in published international application WO95/19978; the compounds disclosed in published international application WO 99/24433 and the compounds disclosed in published international application WO 93/07124.

The pyrazolo [4,3-d]pyrimidin-7-ones disclosed in published international application WO 01/27112; the pyrazolo [4,3-d]pyrimidin-7-ones disclosed in published international application WO 01/27113; the compounds disclosed in EP-A-1092718 and the compounds disclosed in EP-A-1092719.

Preferred type V phosphodiesterase inhibitors for the use according to the present invention include:

5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756);

5-(2-ethoxy-5-morpholinoacetylphenyl)-1-methyl-3-n-propyl-1,6-dihydro-7H-pyrazolo[4,3-d]pyrimidin-7-one (see EP-A-0526004);

3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO98/49 166);

3-ethyl-5-[5-(4-ethylpiperazin-1-ylsulphonyl)-2-(2-methoxyethoxy)pyridin-3-yl]-2-(pyridin-2-yl)methyl-2, 6-dihydro-7H-pyrazolo[4,3-d]pyrimidin-7-one (see WO99/54333);

(+)-3-ethyl-5-[5-(4-ethylpiperazin-1-ylsulphonyl)-2-(2-methoxy-1(R)-methylethoxy)pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1 R)-2-methoxy-1-methylethyl]oxy)pyridin-3-yl}-2-methyl-2,6-dihydro-7H-pyrazolo[4,3-d] pyrimidin-7-one (see WO99/54333);

5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine (see WO 01/27113, Example 8);

5-[2-iso-Butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one (see WO 01/27113, Example 15);

5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO 01/27113, Example 66);

5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one (seeWO 01/27112, Example 124);

5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one (see WO 01/27112, Example 132);

(6R,12aR)-2,3,6,7,12,12a-hexahydro-2-methyl-6-(3,4-methylenedioxyphenyl)-pyrazino[2′,1′:6,1]pyrido[3,4-b]indole-1,4-dione (IC-351), i.e. the compound of examples 78 and 95 of published international application WO95/19978, as well as the compound of examples 1, 3, 7 and 8;

2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil) also known as 1-[[3-(3,4-dihydro-5-methyl-4-oxo-7-propylimidazo[5, 1-f]-as-triazin-2-yl)-4-ethoxyphenyl]sulphonyl]-4-ethylpiperazine, i.e. the compound of examples 20, 19, 337 and 336 of published international application WO99/24433; and

the compound of example 11 of published international application WO93/07124 (EISAI); and

compounds 3 and 14 from Rotella D P, J. Med. Chem., 2000, 43, 1257.

Still other type cGMP PDE5 inhibitors useful in conjunction with the present invention include:4-bromo-5-(pyridylmethylamino)-6-[3-(4-chlorophenyl)-propoxy]-3(2H)pyridazinone; 1-[4-[(1,3-benzodioxol-5-ylmethyl)amiono]-6-chloro-2-quinozolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furazlocillin; cis-2-hexyl-5-methyl-3,4,5,6a,7,8,9,9a-octahydrocyclopent[4,5]-imidazo[2,1-b]purin-4-one; 3-acetyl-1-(2-chlorobenzyl)-2-propylindole-6-carboxylate; 3-acetyl-1-(2-chlorobenzyl)-2-propylindole-6-carboxylate; 4-bromo-5-(3-pyridylmethylamino)-6-(3-(4-chlorophenyl)propoxy)-3-(2H)pyridazinone; 1-methyl-5(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)arnino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); E-8010 and E-4010 (Eisai); Bay-38-3045 & 38-9456 (Bayer) and Sch-51866.

The suitability of any particular cGMP PDE5 inhibitor can be readily determined by evaluation of its potency and selectivity using literature methods followed by evaluation of its toxicity, absorption, metabolism, pharmacokinetics, etc in accordance with standard pharmaceutical practice.

Preferably, the cGMP PDE5 inhibitors have an IC₅₀ at less than 100 nanomolar, more preferably, at less than 50 nanomolar, more preferably still at less than 10 nanomolar.

IC50 values for the cGMP PDE5 inhibitors may be determined using the PDE5 assay in the Test Methods Section hereinafter.

Preferably the cGMP PDE5 inhibitors used in the pharmaceutical combinations according to the present invention are selective for the PDE5 enzyme. Preferably they have a selectivity of PDE5 over PDE3 of greater than 100 more preferably greater than 300. More preferably the PDE5 has a selectivity over both PDE3 and PDE4 of greater than 100, more preferably greater than 300.

Selectivity ratios may readily be determined by the skilled person. IC50 values for the PDE3 and PDE4 enzyme may be determined using established literature methodology, see S A Ballard et al, Journal of Urology, 1998, vol. 159, pages 2164-2171 and as detailed herein after.

It is to be understood that the contents of the above published patent applications, and in particular the general formulae and exemplified compounds therein are incorporated herein in their entirety by reference thereto.

### TREATMENT

It is to be appreciated that all references herein to treatment include one or more of curative, palliative and prophylactic treatment.

### SEXUAL STIMULATION

The present invention also encompasses use as defined hereinbefore via administration of a NEPi (and an PDE5i where applicable) before and/or during sexual stimulation. Here the term “sexual stimulation” may be synonymous with the term “sexual arousal”. This aspect of the present invention is advantageous because it provides systemic selectivity. The natural cascade only occurs at the genitalia and not in other locations—e.g. in the heart etc. Hence, it would be possible to achieve a selective effect on the genitalia via the MED treatment according to the present invention.

Thus, according to the present invention it is highly desirable that there is a sexual stimulation step at some stage. We have found that this step can provide systemic selectivity. Here, “sexual stimulation” may be one or more of a visual stimulation, a physical stimulation, an auditory stimulation, or a thought stimulation.

### ACTIVE AGENT

Agents for use in the treatment of male sexual days function, in particular MED according to of the present invention may be any suitable agent that can act as a NEPi and, where appropriate a combination of a NEPi and a PDE5i, or other additional active agent.

Such agents (i.e. the agents as defined above and/or the additional agents as defined hereinbefore) can be an amino acid sequence or a chemical derivative thereof. The substance may even be an organic compound or other chemical. The agent may even be a nucleotide sequence—which may be a sense sequence or an anti-sense sequence. The agent may even be an antibody.

Thus, the term “agent” includes, but is not limited to, a compound which may be obtainable from or produced by any suitable source, whether natural or not.

The agent may be designed or obtained from a library of compounds which may comprise peptides, as well as other compounds, such as small organic molecules, such as lead compounds.

By way of example, the agent may be a natural substance, a biological macromolecule, or an extract made from biological materials such as bacteria, fungi, or animal (particularly mammalian) cells or tissues, an organic or an inorganic molecule, a synthetic agent, a semi-synthetic agent, a structural or functional mimetic, a peptide, a peptidomimetics, a derivatised agent, a peptide cleaved from a whole protein, or a peptides synthesised synthetically (such as, by way of example, either using a peptide synthesizer or by recombinant techniques or combinations thereof, a recombinant agent, an antibody, a natural or a non-natural agent, a fusion protein or equivalent thereof and mutants, derivatives or combinations thereof.

As used herein, the term “agent” may be a single entity or it may be a combination of agents.

The agent may be in the form of a pharmaceutically acceptable salt—such as an acid addition salt or a base salt—or a solvate thereof, including a hydrate thereof. For a review on suitable salts see Berge et al, J. Pharm. Sci., 1977, 66, 1-19.

Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts.

Suitable base salts are formed from bases which form non-toxic salts and examples are the sodium, potassium, aluminium, calcium, magnesium, zinc and diethanolamine salts.

A pharmaceutically acceptable salt of an agent as defined hereinbefore may be readily prepared by mixing together solutions of the agent and the desired acid or base, as appropriate. The salt may precipitate from solution and be collected by filtration or may be recovered by evaporation of the solvent.

The agent may exisit in polymorphic form.

The agent may contain one or more asymmetric carbon atoms and therefore exists in two or more stereoisomeric forms. Where an agent contains an alkenyl or alkenylene group, cis (E) and trans (Z) isomerism may also occur. The present invention includes the individual stereoisomers of the agent and, where appropriate, the individual tautomeric forms thereof, together with mixtures thereof.

Separation of diastereoisomers or cis and trans isomers may be achieved by conventional techniques, e.g. by fractional crystallisation, chromatography or H.P.L.C. of a stereoisomeric mixture of the agent or a suitable salt or derivative thereof. An individual enantiomer of the agent may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

The present invention also includes all suitable isotopic variations of the agent or a pharmaceutically acceptable salt thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, 35S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent and pharmaceutically acceptable salts thereof can generally be prepared by conventional procedures using appropriate isotopic variations of suitable reagents.

It will be appreciated by those skilled in the art that the agent may be derived from a prodrug. Examples of prodrugs include entities that have certain protected group(s) and which may not possess pharmacological activity as such, but may, in certain instances, be administered (such as orally or parenterally) and thereafter metabolised in the body to form the agent which are pharmacologically active.

It will be further appreciated that certain moieties known as “pro-moieties”, for example as described in “Design of Prodrugs” by H. Bundgaard, Elsevier, 1985 (the disclosured of which is hereby incorporated by reference), may be placed on appropriate functionalities of the agents. Such prodrugs are also included within the scope of the invention.

The term inhibitor as used herein in relation to the NEPi (and where applicable PDE5i compounds) is to be regarded as being interchangeable with the term antagonist. Further the phrase, enhancing the endogenous erectile process, is to be regarded as being interchangeable with the phrase upregulation of the endogenous erectile process.

For some applications (such as a topical application), the agent may also display an ACE (angiotensin converting enzyme) inhibitory action. An ACE assay is presented in the Experimental Section herein. For some applications (such as with particular patient types), such agents (i.e. those that also display ACE inhibitory action) may not be suitable for oral administration.

For some applications, the agent may also display an ECE (endothelium converting enzyme) inhibitory action. ECE assays are well known in the art.

### Pharmaceutical Formulations

The active agents of the invention (i.e. NEPi and combinations thereof, their pharmaceutically acceptable salts, and pharmaceutically acceptable solvates of either entity can be administered alone but, in human therapy will generally be administered in admixture with a suitable pharmaceutical excipient diluent or carrier selected with regard to the intended route of administration and standard pharmaceutical practice.

For example, the compounds of the invention, or salts or solvates thereof can be administered orally, buccally or sublingually in the form of tablets, capsules (including soft gel capsules), ovules, elixirs, solutions or suspensions, which may contain flavouring or colouring agents, for immediate-, delayed-, modified-, or controlled-release such as sustained-, dual-, or pulsatile delivery applications. The compounds of the invention may also be administered via intracavernosal injection. The compounds of the invention may also be administered via fast dispersing or fast dissolving dosages forms or in the form of a high energy dispersion or as coated particles. Suitable pharmaceutical formulations of the compounds of the invention may be in coated or un-coated form as desired.

Such tablets may contain excipients such as microcrystalline cellulose, lactose, sodium citrate, calcium carbonate, dibasic calcium phosphate, glycine and starch (preferably corn, potato or tapioca starch), disintegrants such as sodium starch glycollate, croscarmellose sodium and certain complex silicates, and granulation binders such as polyvinylpyrrolidone, hydroxypropylmethyl cellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate modifying excipients maybe present both within the dosage form i.e. within the matrix, and/or on the dosage form i.e. upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used i.e. where the drug substance is insoluble a fast dispersing dosage form can be prepared and where the drug substance is soluble a fast dissolving dosage form can be prepared.

The compounds of the invention can also be administered parenterally, for example, intracavernosally, intravenously, intra-arterially, intraperitoneally, intrathecally, intraventricularly, intraurethrally intrasternally, intracranially, intramuscularly or subcutaneously, or they may be administered by infusion or needless injection techniques. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

For oral and parenteral administration to human patients, the daily dosage level of the compounds of the invention or salts or solvates thereof will usually be from 10 to 500 mg (in single or divided doses).

Thus, for example, tablets or capsules of the compounds of the invention or salts or solvates thereof may contain from 5 mg to 250 mg of active compound for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention. The skilled person will also appreciate that, for in the treatment of MED according to the present invention, the NEPi (and where appropriate PDE5i or additional agents(s)) compounds may be taken as a single dose on an “as required” basis (i.e. as needed or desired).

### Example Tablet Formulation

In general a tablet formulation could typically contain between about 0.01 mg and 500 mg of compound (or a salt thereof) whilst tablet fill weights may range from 50 mg to 1000 mg. An example formulation for a 10 mg tablet is illustrated:

| Ingredient | % w/w |
|---|---|
| Free acid, Free base or Salt of Compound | 10.000* |
| Lactose | 64.125 |
| Starch | 21.375 |
| Croscarmeliose Sodium | 3.000 |
| Magnesium Stearate | 1.500 |

*This quantity is typically adjusted in accordance with drug activity.

The tablets are manufactured by a standard process, for example, direct compression or a wet or dry granulation process. The tablet cores may be coated with appropriate overcoats.

The compounds/compositions can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A [trade mark] or 1,1,1,2,3,3,3-heptafluoropropane (HFA 227EA [trade mark]), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or “puff” contains from 1 to 50 mg of a compound of the invention for delivery to the patient. The overall daily dose with an aerosol will be in the range of from 1 to 50 mg which may be administered in a single dose or, more usually, in divided doses throughout the day.

The compounds also be formulated for delivery via an atomiser. Formulations for atomiser devices may contain the following ingredients as solubilisers, emulsifiers or suspending agents: water, ethanol, glycerol, propylene glycol, low molecular weight polyethylene glycols, sodium chloride, fluorocarbons, polyethylene glycol ethers, sorbitan trioleate, oleic acid.

Alternatively, the compounds or salts or solvates thereof can be administered in the form of a suppository, or they may be applied topically in the form of a gel, hydrogel, lotion, solution, cream, ointment or dusting powder. The compounds of the invention or salts or solvates thereof may also be dermally administered. The compounds of the invention or salts or solvates thereof may also be transdermally administered, for example, by the use of a skin patch. They may also be administered by the ocular, pulmonary or rectal routes.

For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the compounds or salts or solvates thereof can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compounds may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

Generally, in humans, oral administration of the is the preferred route, being the most convenient in MED, avoiding the well-known disadvantages associated with intracavernosal (i.c.) administration. A preferred oral dosing regimen in MED for a typical man is from about 10 mg to 500 mg of pharmaceutical composition when required. Where the composition comprises the combination of a NEPi and a PDE5I then from 25 mg to 250 mg of each compound may be present. In circumstances where the recipient suffers from a swallowing disorder or from impairment of drug absorption after oral administration, the drug may be administered parenterally, sublingually or buccally.

### PHARMACOKINETICS

### Bioavailability

Preferably, the compounds of the invention(and combinations) are orally bioavailable. Oral bioavailablity refers to the proportion of an orally administered drug that reaches the systemic circulation. The factors that determine oral bioavailability of a drug are dissolution, membrane permeability and metabolic stability. Typically, a screening cascade of firstly in vitro and then in vivo techniques is used to determine oral bioavailablity.

Dissolution, the solubilisation of the drug by the aqueous contents of the gastro-intestinal tract (GIT), can be predicted from in vitro solubility experiments conducted at appropriate pH to mimic the GIT. Preferably the compounds of the invention have a minimum solubility of 50 mcg/ml. Solubility can be determined by standard procedures known in the art such as described in Adv. Drug Deliv. Rev. 23, 3-25, 1997.

Membrane permeability refers to the passage of the compound through the cells of the GIT. Lipophilicity is a key property in predicting this and is defined by in vitro Log D_{7.4} measurements using organic solvents and buffer. Preferably the compounds of the invention have a Log D_{7.4} of −2 to +4, more preferably −1 to +2. The log D can be determined by standard procedures known in the art such as described in J. Pharm. Pharmacol. 1990, 42:144.

Cell monolayer assays such as CaCo₂ add substantially to prediction of favourable membrane permeability in the presence of efflux transporters such as p-glycoprotein, so-called caco-2 flux. Preferably, compounds of the invention have a caco-2 flux of greater than 2×10⁻⁶ cms⁻¹, more preferably greater than 5×10⁻⁶ cms⁻¹. The caco flux value can be determined by standard procedures known in the art such as described in J. Pharm. Sci, 1990, 79, 595-600

Metabolic stability addresses the ability of the GIT or the liver to metabolise compounds during the absorption process: the first pass effect. Assay systems such as microsomes, hepatocytes etc are predictive of metabolic liability. Preferably the compounds of the Examples show metabolic stablity in the assay system that is commensurate with an hepatic extraction of less then 0.5. Examples of assay systems and data manipulation are described in Curr. Opin. Drug Disc. Devel., 201, 4, 36-44, Drug Met. Disp.,2000, 28,1518-1523

Because of the interplay of the above processes further support that a drug will be orally bioavailable in humans can be gained by in vivo experiments in animals. Absolute bioavailability is determined in these studies by administering the compound separately or in mixtures by the oral route. For absolute determinations (% absorbed) the intravenous route is also employed. Examples of the assessment of oral bioavailability in animals can be found in Drug Met. Disp.,2001, 29, 82-87; J. Med Chem, 1997, 40, 827-829, Drug Met. Disp.,1999, 27, 221-226. as described in J. Pharm. Sci 79, 7, p595-600 (1990), and Pharm. Res. vol 14, no. 6 (1997).

### CHEMICAL SYNTHESIS METHODS

Typically the NEPi ,PDE5i and other additional active compounds suitable for the use according to the present invention will be prepared by chemical synthesis techniques.

The agent or target or variants, homologues, derivatives, fragments or mimetics thereof may be produced using chemical methods to synthesize the agent in whole or in part. For example, peptides can be synthesized by solid phase techniques, cleaved from the resin, and purified by preparative high performance liquid chromatography (e.g., Creighton (1983) Proteins Structures And Molecular Principles, W H Freeman and Co, New York N.Y.). The composition of the synthetic peptides may be confirmed by amino acid analysis or sequencing (e.g., the Edman degradation procedure; Creighton, supra).

Direct synthesis of the agent or variants, homologues, derivatives, fragments or mimetics thereof can be performed using various solid-phase techniques (Roberge J Y et al (1995) Science 269: 202-204) and automated synthesis may be achieved, for example, using the ABI 43 1 A Peptide Synthesizer (Perkin Elmer) in accordance with the instructions provided by the manufacturer. Additionally, the amino acid sequences comprising the agent or any part thereof, may be altered during direct synthesis and/or combined using chemical methods with a sequence from other subunits, or any part thereof, to produce a variant agent or target, such as, for example, a variant NEP.

In an alternative embodiment of the invention, the coding sequence of the agent target or variants, homologues, derivatives, fragments or mimetics thereof may be synthesized, in whole or in part, using chemical methods well known in the art (see Caruthers M H et al (1980) Nuc Acids Res Symp Ser 215-23, Horn T et al (1980) Nuc Acids Res Symp Ser 225-232).

### MIMETIC

As used herein, the term “mimetic” relates to any chemical which includes, but is not limited to, a peptide, polypeptide, antibody or other organic chemical which has the same qualitative activity or effect as a reference agent to a target.

### CHEMICAL MODIFICATION

In one embodiment of the present invention, the agent may be a chemically modified agent.

The chemical modification of an agent may either enhance or reduce hydrogen bonding interaction, charge interaction, hydrophobic interaction, Van Der Waals interaction or dipole interaction between the agent and the target.

In one aspect, the identified agent may act as a model (for example, a template) for the development of other compounds.

### RECOMBINANT METHODS

Typically the target for use in the assay of the present invention may be prepared by recombinant DNA techniques.

### AMINO ACID SEQUENCE

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “protein”.

The amino acid sequence may be prepared isolated from a suitable source, or it may be made synthetically or it may be prepared by use of recombinant DNA techniques.

### NUCLEOTIDE SEQUENCE

As used herein, the term “nucleotide sequence” is synonymous with the term “polynucleotide”.

The nucleotide sequence may be DNA or RNA of genomic or synthetic or of recombinant origin. The nucleotide sequence may be double-stranded or single-stranded whether representing the sense or antisense strand or combinations thereof.

For some applications, preferably, the nucleotide sequence is DNA.

For some applications, preferably, the nucleotide sequence is prepared by use of recombinant DNA techniques (e.g. recombinant DNA).

For some applications, preferably, the nucleotide sequence is cDNA.

For some applications, preferably, the nucleotide sequence may be the same as the naturally occurring form for this aspect.

It will be understood by a skilled person that numerous different nucleotide sequences can encode the targets as a result of the degeneracy of the genetic code.

In addition, it is to be understood that skilled persons may, using routine techniques, make nucleotide substitutions that do not substantially affect the activity encoded by the nucleotide sequence of the present invention to reflect the codon usage of any particular host organism in which the target is to be expressed. Thus, the terms “variant”, “homologue” or “derivative” in relation to the nucleotide sequence set out in the attached sequence listings include any substitution of, variation of, modification of, replacement of, deletion of or addition of one (or more) nucleic acid from or to the sequence providing the resultant nucleotide sequence encodes a functional target according the present invention (or even an agent according to the present invention if said agent comprises a nucleotide sequence or an amino acid sequence).

As indicated above, with respect to sequence homology, preferably there is at least 75%, more preferably at least 85%, more preferably at least 90% homology to the NEP sequence cross referenced to herein. More preferably there is at least 95%, more preferably at least 98%, homology. Nucleotide homology comparisons may be conducted as described above. A preferred sequence comparison program is the GCG Wisconsin Besffit program described above. The default scoring matrix has a match value of 10 for each identical nucleotide and −9 for each mismatch. The default gap creation penalty is −50 and the default gap extension penalty is −3 for each nucleotide.

The present invention also encompasses nucleotide sequences that are capable of hybridising selectively to the sequences presented herein, or any variant, fragment or derivative thereof, or to the complement of any of the above. Nucleotide sequences are preferably at least 15 nucleotides in length, more preferably at least 20, 30, 40 or 50 nucleotides in length. These sequences could be used a probes, such as in a diagnostic kit.

### VARIANTS/HOMOLOGUES/DERIVATIVES

In addition to the specific amino acid sequences and nucleotide sequences mentioned herein, the present invention also encompasses the use of variants, homologue and derivatives thereof. Here, the term “homology” can be equated with “identity”.

In the present context, an homologous sequence is taken to include an amino acid sequence which may be at least 75, 85 or 90% identical, preferably at least 95 or 98% identical. In particular, homology should typically be considered with respect to those regions of the sequence known to be essential for an activity. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Besffit package (see below) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux etal, 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8 and tatiana@ncbi.nlm.nih.gov).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | GAP |
| | | ILV |
| | Polar - uncharged | CSTM |
| | | NQ |
| | Polar - charged | DE |
| | | KR |
| AROMATIC | | HFWY |

The present invention also encompasses homologous substitution (substitution and replacement are both used herein to mean the interchange of an existing amino acid residue, with an alternative residue) may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-ε-amino caproic acid#, 7-amino heptanoic acid*, L-methionine sulfone#*, L-norleucine*, L-norvaline*, p-nitro-L-phenylalanine*, L-hydroxyproline#, L-thioproline*, methyl derivatives of phenylalanine (Phe) such as 4-methyl-Phe*, pentamethyl-Phe*, L-Phe (4-amino)#, L-Tyr (methyl)*, L-Phe (4-isopropyl)*, L-Tic (1,2,3,4-tetrahydroisoquinoline-3-carboxyl acid)*, L-diaminopropionic acid# and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell DC, Trends Biotechnol. (1995) 13(4), 132-134.

### HYBRIDISATION

The present invention also encompasses the use of sequences that can hybridise to the target sequences presented herein—such as if the agent is an anti-sense sequence.

The term “hybridization” as used herein shall include “the process by which a strand of nucleic acid joins with a complementary strand through base pairing” as well as the process of amplification as carried out in polymerase chain reaction (PCR) technologies.

Nucleotide sequences of the invention capable of selectively hybridising to the nucleotide sequences presented herein, or to their complement, will be generally at least 75%, preferably at least 85 or 90% and more preferably at least 95% or 98% homologous to the corresponding complementary nucleotide sequences presented herein over a region of at least 20, preferably at least 25 or 30, for instance at least 40, 60 or 100 or more contiguous nucleotides.

The term “selectively hybridizable” means that the nucleotide sequence, when used as a probe, is used under conditions where a target nucleotide sequence is found to hybridize to the probe at a level significantly above background. The background hybridization may occur because of other nucleotide sequences present, for example, in the cDNA or genomic DNA library being screened. In this event, background implies a level of signal generated by interaction between the probe and a non-specific DNA member of the library which is less than 10 fold, preferably less than 100 fold as intense as the specific interaction observed with the target DNA. The intensity of interaction may be measured, for example, by radiolabelling the probe, e.g. with 32P.

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical nucleotide sequences while an intermediate (or low) stringency hybridization can be used to identify or detect similar or related polynucleotide sequences.

In a preferred aspect, the present invention covers nucleotide sequences that can hybridise to the nucleotide sequence of the present invention under stringent conditions (e.g. 65° C. and 0.1×SSC {1×SSC=0.15 M NaCl, 0.015 M Na₃Citrate pH 7.0). Where the nucleotide sequence of the invention is double-stranded, both strands of the duplex, either individually or in combination, are encompassed by the present invention. Where the nucleotide sequence is single-stranded, it is to be understood that the complementary sequence of that nucleotide sequence is also included within the scope of the present invention.

Nucleotide sequences which are not 100% homologous to the sequences of the present invention but fall within the scope of the invention can be obtained in a number of ways. Other variants of the sequences described herein may be obtained for example by probing DNA libraries made from a range of sources. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of the nucleotide sequence set out in herein under conditions of medium to high stringency. Similar considerations apply to obtaining species homologues and allelic variants of the amino acid and/or nucleotide sequences of the present invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

Alternatively, such nucleotide sequences may be obtained by site directed mutagenesis of characterised sequences, such as the nucleotide sequence set out in SEQ ID No 2 of the sequence listings of the present invention. This may be useful where for example silent codon changes are required to sequences to optimise codon preferences for a particular host cell in which the nucleotide sequences are being expressed. Other sequence changes may be desired in order to introduce restriction enzyme recognition sites, or to alter the activity of the protein encoded by the nucleotide sequences.

The nucleotide sequences of the present invention may be used to produce a primer, e.g. a PCR primer, a primer for an alternative amplification reaction, a probe e.g. labelled with a revealing label by conventional means using radioactive or non-radioactive labels, or the nucleotide sequences may be cloned into vectors. Such primers, probes and other fragments will be at least 15, preferably at least 20, for example at least 25, 30 or 40 nucleotides in length, and are also encompassed by the term nucleotide sequence of the invention as used herein.

The nucleotide sequences such as a DNA polynucleotides and probes according to the invention may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Longer nucleotide sequences will generally be produced using recombinant means, for example using a PCR (polymerase chain reaction) cloning techniques. This will involve making a pair of primers (e.g. of about 15 to 30 nucleotides) flanking a region of the targeting sequence which it is desired to clone, bringing the primers into contact with mRNA or cDNA obtained from an animal or human cell, performing a polymerase chain reaction (PCR) under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

Due to the inherent degeneracy of the genetic code, other DNA sequences which encode substantially the same or a functionally equivalent amino acid sequence, may be used to clone and express the target sequences. As will be understood by those of skill in the art, for certain expression systems, it may be advantageous to produce the target sequences with non-naturally occurring codons. Codons preferred by a particular prokaryotic or eukaryotic host (Murray E et al (1989) Nuc Acids Res 17:477-508) can be selected, for example, to increase the rate of the target expression or to produce recombinant RNA transcripts having desirable properties, such as a longer half-life, than transcripts produced from naturally occurring sequence.

### EXPRESSION VECTORS

The nucleotide sequence for use as the target or for expressing the target can be incorporated into a recombinant replicable vector. The vector may be used to replicate and express the nucleotide sequence in and/or from a compatible host cell. Expression may be controlled using control sequences which include promoters/enhancers and other expression regulation signals. Prokaryotic promoters and promoters functional in eukaryotic cells may be used. Tissue specific or stimuli specific promoters may be used. Chimeric promoters may also be used comprising sequence elements from two or more different promoters described above.

The protein produced by a host recombinant cell by expression of the nucleotide sequence may be secreted or may be contained intracellularly depending on the sequence and/or the vector used. The coding sequences can be designed with signal sequences which direct secretion of the substance coding sequences through a particular prokaryotic or eukaryotic cell membrane.

### FUSION PROTEINS

The target amino acid sequence may be produced as a fusion protein, for example to aid in extraction and purification. Examples of fusion protein partners include glutathione-S-transferase (GST), 6×His, GAL4 (DNA binding and/or transcriptional activation domains) and β-galactosidase. It may also be convenient to include a proteolytic cleavage site between the fusion protein partner and the protein sequence of interest to allow removal of fusion protein sequences. Preferably the fusion protein will not hinder the activity of the target.

The fusion protein may comprise an antigen or an antigenic determinant fused to the substance of the present invention. In this embodiment, the fusion protein may be a non-naturally occurring fusion protein comprising a substance which may act as an adjuvant in the sense of providing a generalised stimulation of the immune system. The antigen or antigenic determinant may be attached to either the amino or carboxy terminus of the substance.

In another embodiment of the invention, the amino acid sequence may be ligated to a heterologous sequence to encode a fusion protein. For example, for screening of peptide libraries for agents capable of affecting the substance activity, it may be useful to encode a chimeric substance expressing a heterologous epitope that is recognized by a commercially available antibody.

### ANTIBODIES

In one embodiment of the present invention, the agent may be an antibody. In addition, or in the alternative, the target may be an antibody. In addition, or in the alternative, the means for detecting the target may be an antibody.

Antibodies may be produced by standard techniques, such as by immunisation with the substance of the invention or by using a phage display library.

For the purposes of this invention, the term “antibody”, unless specified to the contrary, includes but is not limited to, polyclonal, monoclonal, chimeric, single chain, Fab fragments, fragments produced by a Fab expression library, as well as mimetics thereof. Such fragments include fragments of whole antibodies which retain their binding activity for a target substance, Fv, F(ab′) and F(ab′)₂ fragments, as well as single chain antibodies (scFv), fusion proteins and other synthetic proteins which comprise the antigen-binding site of the antibody. Furthermore, the antibodies and fragments thereof may be humanised antibodies. Neutralizing antibodies, i.e., those which inhibit biological activity of the substance polypeptides, are especially preferred for diagnostics and therapeutics.

If polyclonal antibodies are desired, a selected mammal (e.g., mouse, rabbit, goat, horse, etc.) is immunised with an immunogenic polypeptide bearing a epitope(s) obtainable from an identified agent and/or substance of the present invention. Depending on the host species, various adjuvants may be used to increase immunological response. Such adjuvants include, but are not limited to, Freund's, mineral gels such as aluminium hydroxide, and surface active substances such as lysolecithin, pluronic polyols, polyanions, peptides, oil emulsions, keyhole limpet hemocyanin, and dinitrophenol. BCG (Bacilli Calmette-Guerin) and Corynebacterium parvum are potentially useful human adjuvants which may be employed if purified the substance polypeptide is administered to immunologically compromised individuals for the purpose of stimulating systemic defence.

Serum from the immunised animal is collected and treated according to known procedures. If serum containing polyclonal antibodies to an epitope obtainable from an identifed agent and/or substance of the present invention contains antibodies to other antigens, the polyclonal antibodies can be purified by immunoaffinity chromatography. Techniques for producing and processing polyclonal antisera are known in the art. In order that such antibodies may be made, the invention also provides polypeptides of the invention or fragments thereof haptenised to another polypeptide for use as immunogens in animals or humans.

Monoclonal antibodies directed against epitopes obtainable from an identifed agent and/or substance of the present invention can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against orbit epitopes can be screened for various properties; i.e., for isotype and epitope affinity.

Monoclonal antibodies to the substance and/or identified agent may be prepared using any technique which provides for the production of antibody molecules by continuous cell lines in culture. These include, but are not limited to, the hybridoma technique originally described by Koehler and Milstein (1975 Nature 256:495-497), the human B-cell hybridoma technique (Kosbor et al (1983) Immunol Today 4:72; Cote et al (1983) Proc Natl Acad Sci 80:2026-2030) and the EBV-hybridoma technique (Cole et al (1985) Monoclonal Antibodies and Cancer Therapy, Alan R Liss Inc, pp 77-96). In addition, techniques developed for the production of “chimeric antibodies”, the splicing of mouse antibody genes to human antibody genes to obtain a molecule with appropriate antigen specificity and biological activity can be used (Morrison et al (1984) Proc Natl Acad Sci 81:6851-6855; Neuberger et al (1984) Nature 312:604-608; Takeda et al (1985) Nature 314:452-454). Alternatively, techniques described for the production of single chain antibodies (U.S. Pat. No. 4,946,779) can be adapted to produce the substance specific single chain antibodies.

Antibodies, both monoclonal and polyclonal, which are directed against epitopes obtainable from an identifed agent and/or substance are particularly useful in diagnosis, and those which are neutralising are useful in passive immunotherapy. Monoclonal antibodies, in particular, may be used to raise anti-idiotype antibodies. Anti-idiotype antibodies are immunoglobulins which carry an “internal image” of the substance and/or agent against which protection is desired. Techniques for raising anti-idiotype antibodies are known in the art. These anti-idiotype antibodies may also be useful in therapy.

Antibodies may also be produced by inducing in vivo production in the lymphocyte population or by screening recombinant immunoglobulin libraries or panels of highly specific binding reagents as disclosed in Orlandi et al (1989, Proc NatI Acad Sci 86: 3833-3837), and Winter G and Milstein C (1991; Nature 349:293-299).

Antibody fragments which contain specific binding sites for the substance may also be generated. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (Huse W D et al (1989) Science 256:1275-128 1).

### REPORTERS

A wide variety of reporters may be used in the assay methods (as well as screens) of the present invention with preferred reporters providing conveniently detectable signals (eg. by spectroscopy). By way of example, a reporter gene may encode an enzyme which catalyses a reaction which alters light absorption properties.

Examples of reporter molecules include but are not limited to β-galactosidase, invertase, green fluorescent protein, luciferase, chloramphenicol, acetyltransferase, β-glucuronidase, exo-glucanase and glucoamylase. Alternatively, radiolabelled or fluorescent tag-labelled nucleotides can be incorporated into nascent transcripts which are then identified when bound to oligonucleotide probes.

In one preferred embodiment, the production of the reporter molecule is measured by the enzymatic activity of the reporter gene product, such as β-galactosidase.

A variety of protocols for detecting and measuring the expression of the target, such as by using either polyclonal or monoclonal antibodies specific for the protein, are known in the art. Examples include enzyme-linked immunosorbent assay (ELISA), radioimmunoassay (RIA) and fluorescent activated cell sorting (FACS). A two-site, monoclonal-based immunoassay utilising monoclonal antibodies reactive to two non-interfering epitopes on polypeptides is preferred, but a competitive binding assay may be employed. These and other assays are described, among other places, in Hampton R et al (1990, Serological Methods, A Laboratory Manual, APS Press, St Paul Minn.) and Maddox D E et al (1983, J Exp Med 15 8:121 1).

A wide variety of labels and conjugation techniques are known by those skilled in the art and can be used in various nucleic and amino acid assays. Means for producing labelled hybridisation or PCR probes for detecting the target polynucleotide sequences include oligolabelling, nick translation, end-labelling or PCR amplification using a labelled nucleotide. Alternatively, the coding sequence, or any portion of it, may be cloned into a vector for the production of an mRNA probe. Such vectors are known in the art, are commercially available, and may be used to synthesize RNA probes in vitro by addition of an appropriate RNA polymerase such as T7, T3 or SP6 and labelled nucleotides.

A number of companies such as Pharmacia Biotech (Piscataway, N.J.), Promega (Madison, Wis.), and US Biochemical Corp (Cleveland, Ohio) supply commercial kits and protocols for these procedures. Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149 and U.S. Pat. No. 4,366,241. Also, recombinant immunoglobulins may be produced as shown in U.S. Pat. No. 4,816,567.

Additional methods to quantify the expression of a particular molecule include radiolabeling (Melby P C et al 1993 J Immunol Methods 159:235-44) or biotinylating (Duplaa C et al 1993 Anal Biochem 229-36) nucleotides, coamplification of a control nucleic acid, and standard curves onto which the experimental results are interpolated. Quantification of multiple samples may be speeded up by running the assay in an ELISA format where the oligomer of interest is presented in various dilutions and a spectrophotometric or calorimetric response gives rapid quantification.

Although the presence/absence of marker gene expression suggests that the gene of interest is also present, its presence and expression should be confirmed. For example, if the nucleotide sequence is inserted within a marker gene sequence, recombinant cells containing the same may be identified by the absence of marker gene function. Alternatively, a marker gene can be placed in tandem with a target coding sequence under the control of a single promoter. Expression of the marker gene in response to induction or selection usually indicates expression of the target as well.

Alternatively, host cells which contain the coding sequence for the target and express the target coding regions may be identified by a variety of procedures known to those of skill in the art. These procedures include, but are not limited to, DNA-DNA or DNA-RNA hybridisation and protein bioassay or immunoassay techniques which include membrane-based, solution-based, or chip-based technologies for the detection and/or quantification of the nucleic acid or protein.

### DIAGNOSTICS

The present invention also provides a diagnostic composition or kit for the detection of a pre-disposition for MED. In this respect, the composition or kit will comprise an entity that is capable of indicating the presence of one or more—or even the absence of one or more—of the targets in a test sample. Preferably, the test sample is obtained from the penis.

By way of example, the diagnostic composition may comprise any one of the nucleotide sequences mentioned herein or a variant, homologue, fragment or derivative thereof, or a sequence capable of hybridising to all or part of any one of the nucleotide sequence.

In order to provide a basis for the diagnosis of disease, normal or standard values from a target should be established. This may be accomplished by combining body fluids or cell extracts taken from normal subjects, either animal or human, with an antibody to a target under conditions suitable for complex formation which are well known in the art. The amount of standard complex formation may be quantified by comparing it to a dilution series of positive controls where a known amount of antibody is combined with known concentrations of a purified target. Then, standard values obtained from normal samples may be compared with values obtained from samples from subjects potentially affected by MED. Deviation between standard and subject values establishes the presence of the disease state.

A target itself, or any part thereof, may provide the basis for a diagnostic and/or a therapeutic compound. For diagnostic purposes, target polynucleotide sequences may be used to detect and quantify gene expression in conditions, disorders or diseases in which MED may be implicated.

The target encoding polynucleotide sequence may be used for the diagnosis of MED resulting from expression of the target. For example, polynucleotide sequences encoding a target may be used in hybridisation or PCR assays of tissues from biopsies or autopsies or biological fluids, to detect abnormalities in target expression. The form of such qualitative or quantitative methods may include Southern or northern analysis, dot blot or other membrane-based technologies; PCR technologies; dip stick, pin or chip technologies; and ELISA or other multiple sample formal technologies. All of these techniques are well known in the art and are in fact the basis of many commercially available diagnostic kits.

Such assays may be tailored to evaluate the efficacy of a particular therapeutic treatment regime and may be used in animal studies, in clinical trials, or in monitoring the treatment of an individual patient. In order to provide a basis for the diagnosis of disease, a normal or standard profile for target expression should be established. This is accomplished by combining body fluids or cell extracts taken from normal subjects, either animal or human, with the target or a portion thereof, under conditions suitable for hybridisation or amplification. Standard hybridisation may be quantified by comparing the values obtained for normal subjects with a dilution series of positive controls run in the same experiment where a known amount of purified target is used. Standard values obtained from normal samples may be compared with values obtained from samples from subjects potentially affected by a disorder or disease related to expression of the target coding sequence. Deviation between standard and subject values establishes the presence of the disease state. If disease is established, an existing therapeutic agent is administered, and treatment profile or values may be generated. Finally, the assay may be repeated on a regular basis to evaluate whether the values progress toward or return to the normal or standard pattern. Successive treatment profiles may be used to show the efficacy of treatment over a period of several days or several months.

Thus, in one aspect, the present invention relates to the use of a target polypeptide, or variant, homologue, fragment or derivative thereof, to produce anti-target antibodies which can, for example, be used diagnostically to detect and quantify target levels in MED.

The present invention further provides diagnostic assays and kits for the detection of a target in cells and tissues comprising a purified target which may be used as a positive control, and anti-target antibodies. Such antibodies may be used in solution-based, membrane-based, or tissue-based technologies to detect any disease state or condition related to the expression of target protein or expression of deletions or a variant, homologue, fragment or derivative thereof.

### ASSAY METHODS

The diagnostic compositions and/or methods and/or kits may be used in the following techniques which include but are not limited to; competitive and non-competitive assays, radioimmunoassay, bioluminescence and chemiluminescence assays, fluorometric assays, sandwich assays, immunoradiometric assays, dot blots, enzyme linked assays including ELISA, microtiter plates, antibody coated strips or dipsticks for rapid monitoring of urine or blood, immunohistochemistry and immunocytochemistry.

By way of example, an immunohistochemistry kit may also be used for localization of NEP activity in genital tissue. This immunohistochemistry kit permits localization of NEP in tissue sections and cultured cells using both light and electron microscopy which may be used for both research and clinical purposes. Such information may be useful for diagnostic and possibly therapeutic purposes in the detection and/or prevention and/or treatment of MED. For each kit the range, sensitivity, precision, reliability, specificity and reproducibility of the assay are established. lntraassay and interassay variation is established at 20%, 50% and 80% points on the standard curves of displacement or activity.

### PROBES

Another aspect of the subject invention is the provision of nucleic acid hybridisation or PCR probes which are capable of detecting (especially those that are capable of selectively selecting) polynucleotide sequences, including genomic sequences, encoding a target coding region or closely related molecules, such as alleles. The specificity of the probe, i.e., whether it is derived from a highly conserved, conserved or non-conserved region or domain, and the stringency of the hybridisation or amplification (high, intermediate or low) will determine whether the probe identifies only naturally occurring target coding sequence, or related sequences. Probes for the detection of related nucleic acid sequences are selected from conserved or highly conserved nucleotide regions of target family members and such probes may be used in a pool of degenerate probes. For the detection of identical nucleic acid sequences, or where maximum specificity is desired, nucleic acid probes are selected from the non-conserved nucleotide regions or unique regions of the target polynucleotides. As used herein, the term “non-conserved nucleotide region” refers to a nucleotide region that is unique to a target coding sequence disclosed herein and does not occur in related family members.

PCR as described in U.S. Pat. No. 4,683,195, U.S. Pat. No. 4,800,195 and U.S. Pat. No. 4,965,188 provides additional uses for oligonucleotides based upon target sequences. Such oligomers are generally chemically synthesized, but they may be generated enzymatically or produced from a recombinant source. Oligomers generally comprise two nucleotide sequences, one with sense orientation (5′->3′) and one with antisense (3′<-5′) employed under optimised conditions for identification of a specific gene or condition. The same two oligomers, nested sets of oligomers, or even a degenerate pool of oligomers may be employed under less stringent conditions for detection and/or quantification of closely related DNA or RNA sequences.

The nucleic acid sequence for a target can also be used to generate hybridisation probes as previously described, for mapping the endogenous genomic sequence. The sequence may be mapped to a particular chromosome or to a specific region of the chromosome using well known techniques. These include in situ hybridisation to chromosomal spreads (Verma et al (1988) Human Chromosomes: A Manual of Basic Techniques, Pergamon Press, New York City), flow-sorted chromosomal preparations, or artificial chromosome constructions such as YACs, bacterial artificial chromosomes (BACs), bacterial PI constructions or single chromosome cDNA libraries.

In situ hybridisation of chromosomal preparations and physical mapping techniques such as linkage analysis using established chromosomal markers are invaluable in extending genetic maps. Examples of genetic maps can be found in Science (1995; 270:410f and 1994; 265:1981f. Often the placement of a gene on the chromosome of another mammalian species may reveal associated markers even if the number or arm of a particular human chromosome is not known. New sequences can be assigned to chromosomal arms, or parts thereof, by physical mapping. This provides valuable information to investigators searching for disease genes using positional cloning or other gene discovery techniques. Once a disease or syndrome has been crudely localised by genetic linkage to a particular genomic region any sequences mapping to that area may represent associated or regulatory genes for further investigation. The nucleotide sequence of the subject invention may also be used to detect differences in the chromosomal location due to translocation, inversion, etc. between normal, carrier or affected individuals.

### ORGANISM

The term “organism” in relation to the present invention includes any organism that could comprise the target and/or products obtained therefrom. Examples of organisms may include a fungus, yeast or a plant.

The term “transgenic organism” in relation to the present invention includes any organism that comprises the target and/or products obtained.

### TRANSFORMATION OF HOST CELLS/HOST ORGANISMS

As indicated earlier, the host organism can be a prokaryotic or a eukaryotic organism. Examples of suitable prokaryotic hosts include E. coli and Bacillus subtilis. Teachings on the transformation of prokaryotic hosts is well documented in the art, for example see Sambrook et al (Molecular Cloning: A Laboratory Manual, 2nd edition, 1989, Cold Spring Harbor Laboratory Press) and Ausubel et al., Current Protocols in Molecular Biology (1995), John Wiley & Sons, Inc.

If a prokaryotic host is used then the nucleotide sequence may need to be suitably modified before transformation—such as by removal of introns.

In another embodiment the transgenic organism can be a yeast. In this regard, yeast have also been widely used as a vehicle for heterologous gene expression. The species Saccharomyces cerevisiae has a long history of industrial use, including its use for heterologous gene expression. Expression of heterologous genes in Saccharomyces cerevisiae has been reviewed by Goodey et al (1987, Yeast Biotechnology, D R Berry et al, eds, pp 401-429, Allen and Unwin, London) and by King et al (1989, Molecular and Cell Biology of Yeasts, E F Walton and G T Yarronton, eds, pp 107-133, Blackie, Glasgow).

For several reasons Saccharomyces cerevisiae is well suited for heterologous gene expression. First, it is non-pathogenic to humans and it is incapable of producing certain endotoxins. Second, it has a long history of safe use following centuries of commercial exploitation for various purposes. This has led to wide public acceptability. Third, the extensive commercial use and research devoted to the organism has resulted in a wealth of knowledge about the genetics and physiology as well as large-scale fermentation characteristics of Saccharomyces cerevisiae.

A review of the principles of heterologous gene expression in Saccharomyces cerevisiae and secretion of gene products is given by E Hinchcliffe E Kenny (1993, “Yeast as a vehicle for the expression of heterologous genes”, Yeasts, Vol 5, Anthony H Rose and J Stuart Harrison, eds, 2nd edition, Academic Press Ltd.).

Several types of yeast vectors are available, including integrative vectors, which require recombination with the host genome for their maintenance, and autonomously replicating plasmid vectors.

In order to prepare the transgenic Saccharomyces, expression constructs are prepared by inserting the nucleotide sequence of the present invention into a construct designed for expression in yeast. Several types of constructs used for heterologous expression have been developed. The constructs contain a promoter active in yeast fused to the nucleotide sequence of the present invention, usually a promoter of yeast origin, such as the GAL1 promoter, is used. Usually a signal sequence of yeast origin, such as the sequence encoding the SUC2 signal peptide, is used. A terminator active in yeast ends the expression system.

For the transformation of yeast several transformation protocols have been developed. For example, a transgenic Saccharomyces according to the present invention can be prepared by following the teachings of Hinnen et al (1978, Proceedings of the National Academy of Sciences of the USA 75, 1929); Beggs, J D (1978, Nature, London, 275, 104); and Ito, H et al (1983, J Bacteriology 153, 163-168).

The transformed yeast cells are selected using various selective markers. Among the markers used for transformation are a number of auxotrophic markers such as LEU2, HIS4 and TRP1, and dominant antibiotic resistance markers such as aminoglycoside antibiotic markers, eg G418.

Another host organism is a plant. The basic principle in the construction of genetically modified plants is to insert genetic information in the plant genome so as to obtain a stable maintenance of the inserted genetic material. Several techniques exist for inserting the genetic information, the two main principles being direct introduction of the genetic information and introduction of the genetic information by use of a vector system. A review of the general techniques may be found in articles by Potrykus (Annu Rev Plant Physiol Plant Mol Biol [1991] 42:205-225) and Christou (Agro-Food-Industry Hi-Tech March/April 1994 17-27). Further teachings on plant transformation may be found in EP-A-0449375.

Thus, the present invention also provides a method of transforming a host cell with a nucleotide sequence that is to be the target or is to express the target. Host cells transformed with the nucleotide sequence may be cultured under conditions suitable for the expression and recovery of the encoded protein from cell culture. The protein produced by a recombinant cell may be secreted or may be contained intracellularly depending on the sequence and/or the vector used. As will be understood by those of skill in the art, expression vectors containing coding sequences can be designed with signal sequences which direct secretion of the coding sequences through a particular prokaryotic or eukaryotic cell membrane. Other recombinant constructions may join the coding sequence to nucleotide sequence encoding a polypeptide domain which will facilitate purification of soluble proteins (Kroll D J et al (1993) DNA Cell Biol 12:441-53).

### NEPi—ANIMAL TEST METHODS

### Animal models

### Anaesthetised Rabbit Methodologv

Male New Zealand rabbits (˜2.5kg) were pre-medicated with a combination of Medetomidine (Domitor®) 0.5 ml/kg i.m., and Ketamine (Vetalar®) 0.25 ml/kg i.m. whilst maintaining oxygen intake via a face mask. The rabbits were tracheotomised using a Portex™ uncuffed endotracheal tube 3 ID., connected to ventilator and maintained at a ventilation rate of 30-40 breaths per minute, with an approximate tidal volume of 18-20 ml, and a maximum airway pressure of 10 cm H₂O. Anaesthesia was then switched to Isoflurane and ventilation continued with O₂ at 2 l/min. The right marginal ear vein was cannulated using a 23G or 24G catheter, and Lactated Ringer solution perfused at 0.5 ml/min. The rabbit was maintained at 3% Isoflurane during invasive surgery, dropping to 2% for maintenance anaesthesia. The left jugular vein was exposed, isolated and then cannulated with a PVC catheter (17G) for the infusion of drugs and compounds.

The left groin area of the rabbit was shaved and a vertical incision was made approximately 5 cm in length along the thigh. The femoral vein and artery were exposed, isolated and then cannulated with a PVC catheter (17G) for the infusion of drugs and compounds. Cannulation was repeated for the femoral artery, inserting the catheter to a depth of 1 Ocm to ensure that the catheter reached the abdominal aorta. This arterial catheter was linked to a Gould system to record blood pressure. Samples for blood gas analysis were also taken via the arterial catheter. Systolic and diastolic pressures were measured, and the mean arterial pressure calculated using the formula (diastolic ×2+systolic)÷3. Heart rate was measured via the pulse oxymeter and Po-ne-mah data acquisition software system (Ponemah Physiology Platform, Gould Instrument Systems Inc).

A ventral midline incision was made into the abdominal cavity. The incision was about 5 cm in length just above the pubis. The fat and muscle was bluntly dissected away to reveal the hypogastric nerve which runs down the body cavity. It was essential to keep close to the side curve of the pubis wall in order to avoid damaging the femoral vein and artery which lie above the pubis. The sciatic and pelvic nerves lie deeper and were located after further dissection on the dorsal side of the rabbit. Once the sciatic nerve is identified, the pelvic nerve was easily located. The term pelvic nerve is loosely applied; anatomy books on the subject fail to identify the nerves in sufficient detail. However, stimulation of the nerve causes an increase in intracavernosal pressure and cavernosal blood flow, and innervation of the pelvic region. The pelvic nerve was freed away from surrounding tissue and a Harvard bipolar stimulating electrode was placed around the nerve. The nerve was slightly lifted to give some tension, then the electrode was secured in position. Approximately 1 ml of light paraffin oil was placed around the nerve and electrode. This acts as a protective lubricant to the nerve and prevents blood contamination of the electrode. The electrode was connected to a Grass S88 Stimulator. The pelvic nerve was stimulated using the following parameters:—5V, pulse width 0.5 ms, duration of stimulus 20 seconds with a frequency of 16Hz. Reproducible responses were obtained when the nerve was stimulated every 15-20 minutes. Several stimulations using the above parameters were performed to establish a mean control response. The compound(s) to be tested were infused, via the jugular vein, using a Harvard 22 infusion pump allowing a continuous 15 minute stimulation cycle. The skin and connective tissue around the penis was removed to expose the penis. A catheter set (Insyte-W, Becton-Dickinson 20 Gauge 1.1×48 mm) was inserted through the tunica albica into the left corpus cavernosal space and the needle removed, leaving a flexible catheter. This catheter was linked via a pressure transducer (Ohmeda 5299-04) to a Gould system to record intracavernosal pressure. Once an intracavernosal pressure was established, the catheter was sealed in place using Vetbond (tissue adhesive, 3M). Heart rate was measured via the pulse oxymeter and Po-ne-mah data acquisition software system (Ponemah Physiology Platform, Gould Instrument Systems Inc).

Intracavernosal blood flow was recorded either as numbers directly from the Flowmeter using Po-ne-mah data acquisition software (Ponemah Physiology Platform, Gould Instrument Systems Inc), or indirectly from Gould chart recorder trace. Calibration was set at the beginning of the experiment (0-125 ml/min/100 g tissue). The NEP (Neutral Endopeptidase EC3.4.24.11) inhibitor was made up in saline+10% 1M NaOH, the phosphodiesterase type 5 (PDE5) inhibitor was made up in saline+5% 1M HCl. The inhibitors and vehicle controls were infused at a rate of 0.1 ml/second. NEP inhibitors and PDE_{cAMp} inhibitors were left for 15 minutes prior to pelvic nerve stimulation.

All data are reported as mean±s.e.m. Significant changes were identified using Student's t-tests.

### Anaesthetised Dog Methodology

Male beagles, in the range 12-15 kg body weight, were deprived of food overnight. They were anaesthetised with pentobarbitone (30-45 mg/kg i.v.), and the anaesthesia maintained by a continual infusion of pentobarbitone (60 mg/ml) at a rate of 1-1.4 ml/h. The left femoral artery was cannulated for the measurement of blood pressure, lead II E.C.G. was recorded and heart-rate derived. A catheter was introduced into the left femoral vein for the administration of compounds. Both ureters were cannulated via a mid-line abdominal incision to prevent urine accumulation in the bladder and the bladder was completely emptied. The left internal pudendal artery was carefully dissected free of surrounding tissues to allow placement of a Transonic flow probe for the measurement of arterial blood flow. The cavernosal branches of both pelvic nerves were dissected free and placed into bipolar stimulating electrodes. The skin around the penis was opened and the corpora cavernosa exposed. A 21 g needle, attached by flexible catheter to a pressure transducer, was inserted into the corpus (usually the left) for measurement of both i.c. pressure and injection of SNP; the system was filled with heparinised saline (15 to 20 U/ml). In the dog the corpora are totally separate which enabled either or both sides to be used if necessary.

The dogs were respired with a Ugo Basile 5025 dog ventilator adjusted to maintain blood gasses in the range pO₂ 95-115 mmHg; pCO₂ 25-40 mmHg. Expired air was continually monitored by a Datex Normocap 200 to aid respiratory control. Body temperature was maintained within the range 36-38° C. using an electric blanket. Parameters were recorded on a Gould TA4000 polygraph and all data acquisition and calculation of derived parameters was carried out on-line using a Po-Ne-Mah system. The cavernosal branches of the pelvic nerves were stimulated with a Grass S88 stimulator at 10 volts, 2 ms duration for <1 min. Following a period of equilibration, the pelvic nerves were stimulated at 16 Hz in order to assess whether the rise in i.c. pressure was rapidly and fully registered by the transducer and changes in blood flow were detected. Control responses were obtained to nerve stimulation at either 1 or 2 Hz, On recovery a second stimulation was performed, at double the first frequency. In some dogs a third frequency was used. This stimulation cycle was repeated after 30 min. NEP inhibitors were dissolved in alkaline saline and given as a series of two-tiered infusions starting with a loading infusion and a maintenance infusion for 30 minutes, when the second set of infusions was started. Subsequent infusions were started either at 30 min intervals or when ic. pressure had returned to baseline. All Infusions were given at a rate of 1 ml/min. Stimulation cycles were started fifteen minutes into each infusion.

In addition, arterial blood samples were taken from the abdominal aorta, via the blood pressure cannula, pre-dose and at 15 and 30minutes into each infusion, for subsequent analysis of unbound compound concentration by Drug Metabolism.

### NEPi—TEST RESULTS and DISCUSSION

There are a number of anaesthetised animal models of erection which mimic the physiology of penile erection, i.e. increases in penile blood flow and intracavernosal pressure. The effects of sexual arousal are mimicked by stimulation of pelvic neurones that innervate the penis. This is a mechanism to investigate erectile mechanisms and to assess potential therapeutic agents for the treatment of MED.

It is now established that selective PDE5 inhibitors such as sildenafil enhance nerve stimulated-increases in intracavernosal pressure (ICP) in animal models and that nerve stimulation mimics the erectile process observed in man (Carter et al., 1998, Traish et al., 1999, Omote 1999, Wallis 1999). This PDE5 inhibitor-induced enhancement of ICP characterises the mechanism of action of PDE5 inhibitors and explains how agents such as sildenafil overcomes any relaxant deficiencies associated with MED or impotence. In agreement with these previous studies, the examples hereinafter have demonstrated that a selective PDE5 inhibitor, administered intravenously, potentiates nerve-stimulated increases in ICP in the anaesthetised rabbit and dog (Examples 2, 4, 5).

The examples hereinafter demonstrate that inhibition of NEP EC3.4.24.11 with a selective NEP inhibitor dose-dependently potentiates nerve stimulated increases in intracavernosal pressure in the anaesthetised dog (Examples 1, 2 and 3). At the doses used in this study a similar enhancement of the erectile process was observed with a NEPi as was observed with a PDE5 inhibitor (Example 2). Simultaneously recording intracavernosal pressure (ICP) and cavernosal blood flow illustrated that a selective NEP inhibitor enhanced both ICP and cavernosal blood flow (Example 3). These examples underline the potential clinical application of a NEP inhibitor therapy to enhance the erectile process and hence in the treatment of MED.

Examples 4 and 5 demonstrate that concomitant inhibition of NEP EC3.4.24.11 and PDE5 produced a marked enhancement of the ICP, or the erectile process, than was achievable with the same dose of the same PDE5 inhibitor alone. Using the rabbit model of erection, it has been demonstrated that the potentiation of ICP induced by PDE5 inhibition can be further potentiated by co-administration of a NEP EC3.4.24.11 inhibitor (via intravenous administration of a NEPi, 1 mg/kg; Example 4). At 1 mg/kg (iv) doses of PDE5 inhibitor we observe a maximal potentiation of ICP, the finding that the ICP can be further potentiated beyond this maximal PDE5 inhibitor mediated is highly unexpected. This data illustrates that there are a number of clinical benefits of concomitant administration of a PDE5 inhibitor and a NEP inhibitor over PDE5 inhibitor therapy alone. These include increased efficacy and opportunities to treat MED subgroups that do not respond to PDE5 inhibitor therapy.

A preferred aspect of the present invention provides pharmaceutical compositions comprising a NEPi and a PDEi for use in the treatment of MED wherein the specific combination provides synergistic benefits.

In addition the onset of action of PDE5 inhibitors i.e. the time taken to reach maximal effect is greatly reduced in the presence of a NEP EC 3.4.24.11 inhibitor (Example 5). Clinically this represents a quicker onset time.

In addition, co-administration of a NEPi and a PDE5i allows the onset of action of PDE5i to be reduced. Hence there is a quickening of the time between agent administration and clinical endpoint.

Inhibitors of NEP EC3.4.24.11 and PDE5 or combinations of the two, have no significant effect on un-stimulated ICP i.e. they do not directly induce an increase in ICP in the absence of sexual drive/arousal. This is highly advantageous as the only other marketed therapy for MED which requires sexual stimulation to work is sildenafil thus the present invention provides a viable alternative oral therapy to sildenafil and all other PDE5 alone based drugs.

### NEPi—ANIMAL MODEL EXAMPLES

Compounds used in Examples 1 to 6:

NEPi: see (2R)-2-[(1-{[(5-ethyl-1,3,4-thiadiazol-2-yl)amino]carbonyl}cyclopentyl) methyl]pentanoic acid (preparation herein);IC50 against human native NEP=18.9 nM, selectivity for NEP (human) over ACE (native human) is greater than 500, and NEP selectivity over ECE (recombinant) is greater than 3000.

PDE5i: 3-ethyl-5-{5-[4-ethylpiperzino)sulphonyl-2-propoxyphenyl}-2-(2-pyridylmethyl)-6,7-dihydro-2H-pyrazolo[4,3-d]pyrimidin-7-one also known as 3-ethyl-5-[5-(4-ethylpiperazin-1-ylsulphonyl)-2-n-propoxyphrenyl]-2-(pyridin-2-yl)methyl-2,6-dihydro-7H-pyrazolo[4,3-d]pyrimidin-7-one (see WO98/491066). IC50 against human native PDE5=1.1 nM, selectivity for PDE5 over PDE3 (both on native human) is greater than 90,000 and selectivity over PDE4 is 18545.

All potency and selectivity values quoted are with respect to the human native enzyme (see assays herein).

### Example 1

### Inhibition of NEP EC3.4.24.11 Dose-Dependently Potentiates Nerve Stimulated Increases in Intracavernosal Pressure in Anaesthetised Dog Model of Erection

Submaximal increases in intracavernosal pressure (ICP) induced by nerve-stimulation were significantly increased in the presence of increasing doses of a selective NEP EC3.4.24.11 inhibitor (iv infusion to steady state concentrations). The maximal potentiation (circa 70%) was observed at around 10 times the IC50 value obtained against native NEP. Data is expressed as the percentage (%) increase, compared to control stimulated increases, in ICP divided by mean blood pressure (MBP) and multiplied by 100. Values are expressed as mean±s.e.mean. * P<0.01, Students t-test unpaired compared with control increases.

There were no major effects of NEP inhibition on basal/un-stimulated intracavernosal pressure.

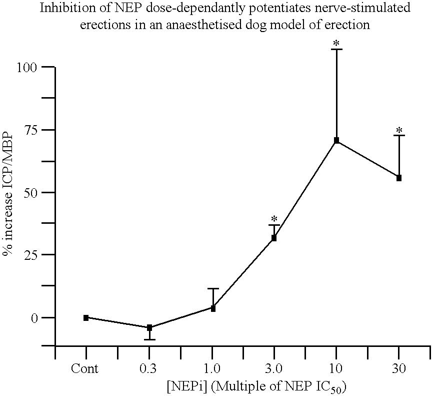

### Example 2

### Inhibition of PDE5 or NEP EC3.4.24.11 Potentiates Nerve Stimulated Increases in Intracavernosal Pressure in Anaesthetised Dog Model of Erection

Submaximal increases in intracavernosal pressure (ICP) induced by nerve-stimulation were significantly increased in the presence of a selective PDE5 inhibitor (10 μg/kg; iv bolus) NEP EC3.4.24.11 inhibitor (100 μg/kg; iv bolus). The maximal potentiation for both agents was circa 65% at the doses used. Data is expressed as ICP divided by mean blood pressure (MBP) and multiplied by 100. Values are expressed as mean±s.e.mean. * P<0.01, Students t-test unpaired compared with control increases.

There were no major effects of NEP or PDE5 inhibition on basal/un-stimulated intracavernosal pressure.

[chemical structure]

### Example 3

### NEP Inhibition Dose-Dependently Potentiates Nerve Stimulated Increases in Intracavernosal Pressure and Cavernosal Blood Flow in Anaesthetised Dog Model of Erection

Submaximal increases in intracavernosal pressure (ICP) and increases in cavernosal blood flow induced by nerve-stimulation were increased in the presence of increasing doses of a selective NEP EC3.4.24.11 inhibitor (iv infusion to steady state concentrations). ICP was increased circa 188% whereas flow was increased circa 228%. Data for ICP and flow, both expressed as area under the curve (AUC), were recorded simultaneously from a single animal.

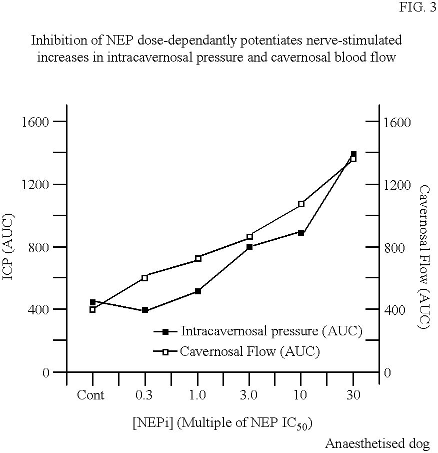

### Example 4

### NEP Inhibition Significantly Increases the Efficacy of PDE5 Inhibitor to Enhance Penile Erection in an Anaesthetised Rabbit Model of Erection

Intravenous administration of a selective PDE5 inhibitor (1 mg/kg) significantly enhanced nerve-stimulated increases in ICP by 133±22% compared to control increases. Once the PDE5i-mediated increase was sustained, co-administration of a selective NEP EC3.4.24.11 inhibitor further enhanced nerve-stimulated increases in ICP. This represents a NEP inhibition-induced potentiated of 79% (P<0.01, paired t-test) compared to increases observed with a PDE5 inhibitor. Data is expressed as percentage increase in ICP over control increases. Values are expressed as mean±s.e.mean. * P<0.01, Students t-test unpaired compared with control increases. There were no effects of PDE5 inhibition or combined PDE5/NEP inhibition on basal/un-stimulated intracavernosal pressure.

[chemical structure]

### Example 5

### NEP Inhibition Potentiates the Erectile Effects of PDE5 Inhibitors and Speeds Up the Onset of Action of PDE5 Inhibitors in the Anaesthetised Rabbit Model of Erection

Concomitant inhibition of NEP EC3.4.24.11 and PDE5 significantly potentiates the PDE5 inhibitor-mediated enhancement of nerve-stimulated increases in intracavernosal pressure (ICP). Submaximal increases in ICP are significantly enhanced (circa 90% compared to control increases) in the presence of a selective PDE5 inhibitor (1 mg/kg; iv bolus). When the same dose of the PDE5 inhibitor is given in the presence of a NEP inhibitor (1 mg/kg; iv bolus) a further enhancement of ICP is observed (circa 187% compared to control increases). This represents a NEP inhibitor mediated enhance of PDE5 inhibitor mediated effects of around 100%.

In addition to the increased enhancement of ICP observed on concomitant application of a NEPi and a PDE5i, the time taken for a PDE5 inhibitor to exert it's maximal effect (i.e. onset of action) is reduced in the presence of a NEP inhibitor (22.5 min in the presence compared to 67.5 min in the absence of a NEP inhibitor).

There were no major effects of NEP inhibition or combined PDE5/NEP inhibition on basal/un-stimulated intracavernosal pressure.

Data is expressed as percentage increase in ICP over control increases. Values are expressed as mean±s.e.mean. * P<0.01, Students t-test unpaired compared with PDE5 inhibitor mediated increases.

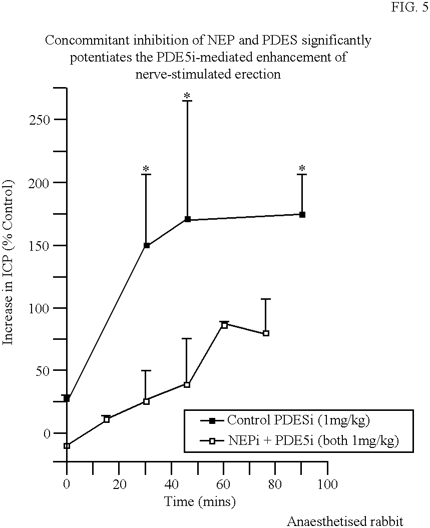

### Example 6

### Effect of Agents that Enhance Intracavernosal Pressure on the Mean Arterial Blood Pressure in the Anaesthetised Rabbit

In the search for novel therapies to treat male sexual dysfunctions such as MED it is desirable that there are no associated adverse cardiovascular effects eg effect on blood pressure or heart rate. In our studies, we have found that infusions of VIP significantly reduce mean arterial blood pressure (See. 6 ) and significantly increased heart rate. Inhibitors of PDE5 (1 mg/kg) and NEP (1 mg/kg), or a concomitant application of a NEP inhibitor and a PDE5 inhibitor (both at 1 mg/kg) however had no substantial effect on blood pressure or heart rate.

FIG. 6 —Intravenous administration of a NEPi, a PDE5i or a concomitant application of a NEPi with a PDE5i had no substantial effect the mean arterial blood pressure in the anaesthetised rabbit model of penile erection. This graph illustrates the typical effects of VIP, a vasoactive agent, a NEP inhibitor (1 mg/kg), or a concomitant application of a NEP inhibitor and a PDE5 inhibitor (both at 1 mglkg) on mean arterial pressure in the anaesthetised rabbit. These observed effects are typical of the trends seen in all animals tested. VIP induced a significant depression of mean arterial pressure (circa 6 mmHg) whereas control infusions of Hepsaline or inhibitors of PDE5 or NEP have no effect on blood pressure. Note, the reduction in blood pressure associated with VIP infusions is also associated with a large increase in heart rate.

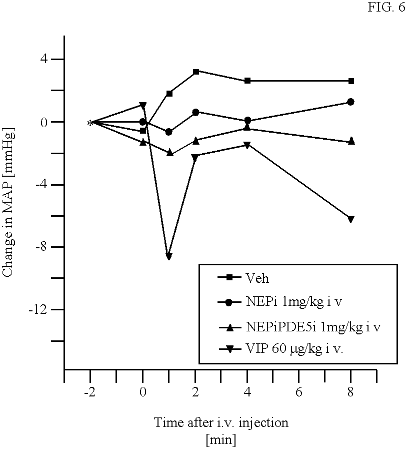

### SCREENS

Any one or more of appropriate targets—such as an amino acid sequence and/or nucleotide sequence—may be used for identifying a NEPi in any of a variety of drug screening techniques. The target employed in such a test may be free in solution, affixed to a solid support, borne on a cell surface, or located intracellularly. The target may even be within an animal model, wherein said target may be an exogenous target or an introduced target. The animal model will be a non-human animal model. The abolition of target activity or the formation of binding complexes between the target and the agent being tested may be measured.

Techniques for drug screening may be based on the method described in Geysen, European Patent Application 84/03564, published on Sep. 13, 1984. In summary, large numbers of different small peptide test compounds are synthesized on a solid substrate, such as plastic pins or some other surface. The peptide test compounds are reacted with a suitable target or fragment thereof and washed. Bound entities are then detected—such as by appropriately adapting methods well known in the art. A purified target can also be coated directly onto plates for use in a drug screening techniques. Alternatively, non-neutralising antibodies can be used to capture the peptide and immobilise it on a solid support.

This invention also contemplates the use of competitive drug screening assays in which neutralising antibodies capable of binding a target specifically compete with a test compound for binding to a target.

Another technique for screening provides for high throughput screening (HTS) of agents having suitable binding affinity to the substances and is based upon the method described in detail in WO 84/03564.

It is expected that the assay methods of the present invention will be suitable for both small and large-scale screening of test compounds as well as in quantitative assays.

In a preferred aspect, the screen of the present invention comprises at least the following steps (which need not be in this same consecutive order): (a) conducting an in vitro screen to determine whether a candidate agent has the relevant activity (such as modulation of NEP, such as NEP from dog kidney); (b) conducting one or more selectivity screens to determine the selectivity of said candidate agent (e.g. to see if said agent is also an ACE inhibitor—such as by using the assay protocol presented herein); and (c) conducting an in vivo screen with said candidate agent (e.g. using a functional animal model). Typically, if said candidate agent passes screen (a) and screen (b) then screen (c) is performed.

### NEP ENZYME ASSAY

NEPi potency figures referred to herein are determined by the following assay.

### THE PREPARATION AND ASSAY OF SOLUBLE (NEP) NEUTRAL ENDOPEPTIDASE FROM CANINE, RAT, RABBIT AND HUMAN KIDNEY CORTEX

Soluble NEP is obtained from the kidney cortex and activity is assayed by measuring the rate of cleavage of the NEP substrate Abz-D-Arg-Arg-Leu-EDDnp to generate its fluorescent product, Abz-D-Arg-Arg.

### EXPERIMENTAL PROCEDURE

1. MATERIALS

All water is double de ionised.

1.1 Tissues

Human Kidney IIAM (Pennsylvania. U.S.A.)

Rat Kidney

Rabbit Kidney

Canine Kidney

1.2 Homogenisation medium

100 mM Mannitol and 20 mM Tris @ pH 7.1

2.42 g Tris (Fisher T/P630/60) is diluted in 1 liter of water and the pH adjusted to 7.1 using 6M HCl at room temperature. To this 18.22 g Mannitol (Sigma M-9546) is added.

1.3 Tris buffer (NEP buffer).

50 ml of 50 mM Tris pH 7.4 (Sigma T2663) is diluted in 950 ml of water.

1.4 Substrate (Abz-D-Arg-Arg-Leu-EDDnp)

Made to order from SNPE, and is stored as a powder at −20° C. A 2 mM stock is made by gently re-suspending the substrate in Tris buffer, this should not be vortexed or sonicated. 600 μl aliquots of the 2 mM stock are stored at −20 for up to one month. (Medeiros, M. A. S., Franca, M. S. F. et al., (1997), Brazilian Journal of Medical and Biological Research, 30, 1157-1162).

1.5 Total product

Samples corresponding to 100% substrate to product conversion are included on the plate to enable the % substrate turnover to be determined. The total product is generated by incubating 1 ml of 2 mM substrate with 20 μl of enzyme stock for 24 hours at 37° C.

1.6 Stop solution.

A 300 μM stock of Phosphoramidon (Sigma R7385) is made up in NEP buffer and stored in 50 μl aliquots at −20.

1.7 Dimethyl sulphoxide (DMSO).

1.8 Magnesium Chloride —MgCl₂.6H₂O (Fisher M0600/53).

1.9 Black 96 well flat bottom assay plates (Costar 3915).

1.10 Topseal A (Packard 6005185).

1.11 Centrifuge tubes

2. SPECIFIC EQUIPTMENT

2.1 Sorvall RC-5B centrifuge (SS34 GSA rotor, pre-cooled to 4° C.).

2.2 Braun miniprimer mixer.

2.3 Beckman CS-6R centrifuge.

2.4 Fluostar galaxy.

2.5 Wesbart 1589 shaking incubator.

3. METHODS

3.1 TISSUE PREPARATION

3.2 Dog, rat, rabbit, and human NEP is obtained from the kidney cortex using a method adapted from Booth, A. G. & Kenny, A. J. (1974) Biochem. J. 142, 575-581.

3.3 Frozen kidneys are allowed to thaw at room temperature and the cortex is dissected away from the medulla.

3.4 The cortex is finely chopped and homogenised in approximately 10 volumes of homogenisation buffer (1.2) using a Braun miniprimer (2.2).

3.5 Magnesium chloride (1.8) (20.3 mg/gm tissue) is added to the homogenate and stirred in an ice-water bath for 15 minutes.

3.6 The homogenate is centrifuged at 1,500 g (3,820 rpm) for 12 minutes in a Beckman centrifuge (2.3) before removing the supernatant to a fresh centrifuge tube and discarding the pellet.

3.7 The supernatant is centrifuged at 15,000 g (12,100 rpm) for 12 minutes in a Sovall centrifuge (2.1) and the supernatant is discarded.

3.8 The pale pink layer on the top of the remaining pellet is removed and re-suspended in homogenisation buffer containing magnesium chloride (9 mg MgCl in 5 ml buffer per 1 g tissue).

3.9 The suspension is centrifuged at 2,200 g (4,630 rpm) for 12 minutes in a Beckman centrifuge (2.3) before discarding the pellet.

3.10 The supernatant is centrifuged at 15,000 g (12,100 rpm) for 12 minutes using the Sorvall centrifuge (2.1) and the supernatant is discarded.

3.11 The final pellet is resuspended in homogenisation buffer containing magnesium chloride (0.9 mg MgCl in 0.5 ml buffer per 1 g tissue). A homogenous suspension is obtained using a Braun miniprimer (2.2). This is then frozen down in 100 μl aliquots to be assayed for NEP activity.

4.0 DETERMINATION OF NEP ACTIVITY

The activity of the previously aliquoted NEP is measured by its ability to cleave the NEP specific peptide substrate.

4.1 A 4% DMSO/NEP buffer solution is made (4 mls DMSO in 96 mls NEP buffer).

4.2 Substrate, total product, enzyme, and Phosphoramidon stocks are left on ice to thaw.

4.3 50 μl of 4% DMSO/NEP buffer solution is added to each well.

4.4 The 2 mM substrate stock is diluted 1:40 to make a 50 μM solution. 100 μl of 50 μM substrate is added to each well (final concentration 25 μM).

4.5 50 μl of a range of enzyme dilutions is added to initiate the reaction (usually 1:100, 1:200, 1:400, 1:800, 1:1600, and 1:3200 are used). 50 μl of NEP buffer is added to blank wells.

4.6 The 2 mM total product is diluted 1:80 to make a 25 μM solution. 200 μl of 25 μM product is added to the first four wells of a new plate.

4.7 Plates are incubated at 37° C. in a shaking incubator for 60 minutes.

4.8 The 300 μM Phosphoramidon stock is diluted 1:100 to 300 nM. The reaction is stopped by the addition of 100 μl 300 nM Phosphoramidon and incubated at 37° C. in a shaking incubator for 20 minutes before being read on the Fluostar (ex320/em420).

5. NEP INHIBITION ASSAYS

5.1 Substrate, total product, enzyme and Phoshoramidon stocks are left on ice to thaw.

5.2 Compound stocks are made up in 100% DMSO and diluted 1:25 in NEP buffer to give a 4% DMSO solution. All further dilutions are carried out in a 4% DMSO solution (4 mls DMSO in 96 mls NEP buffer).

5.3 50 μl of compound in duplicate is added to the 96 well plate and 50 μl of 4% DMSO/NEP buffer is added to control and blank wells.

5.4 The 2 mM substrate stock is diluted 1:40 in NEP buffer to make a 50 μM solution (275 μl 2 mM substrate to 10.73 ml buffer is enough for 1 plate).

5.5 The enzyme stock diluted in NEP buffer (determined from activity checks).

5.6 The 2 mM total product stock is diluted 1:80 in NEP buffer to make a 25 μM solution. 200 μl is added to the first four wells of a separate plate.

5.7 The 300 μM Phosphoramidon stock is diluted 1:1000 to make a 300 nM stock (11 μl Phosphoramidon to 10.99 ml NEP buffer.

5.8 To each well in the 96 well plate the following is added:

TABLE
Reagents to be added to 96 well plate.

| | Compound/ | | Tris | | | NEP | Total |
|---|---|---|---|---|---|---|---|
| | DMSO | | Buffer | | Substrate | enzyme | product |
| Samples | 2 μl | compound | 50 | μl | 100 μl | 50 μl | None |
| Controls | 2 μl | DMSO | 50 | μl | 100 μl | 50 μl | None |
| Blanks | 2 μl | DMSO | 100 | μl | 100 μl | None | None |

| | | | | | | |
|---|---|---|---|---|---|---|
| Totals | 2 μl | DMSO | None | None | None | 200 μl |

5.9 The reaction is initiated by the addition of the NEP enzyme before incubating at 37° C. for 1 hour in a shaking incubator.

5.10 The reaction is stopped with 100 μl 300 nM Phosphoramidon and incubated at 37° C. for 20 minutes in a shaking incubator before being read on the Fluostar (ex320/em420).

6. CALCULATIONS

The activity of the NEP enzyme is determined in the presence and absence of compound and expressed as a percentage.

% Control activity (turnover of enzyme):

$$\frac{Mean FU of controls - Mean FU of blanks}{Mean FU of totals - Mean FU of blanks} \times 100$$

% Activity with inhibitor:

$$\frac{Mean FU of compound - Mean FU of blanks}{Mean FU to totals - Mean FU of blanks} \times 100$$

Activity expressed as % of control:

$$\frac{\% Activity with inhibitor}{\% Control activity} \times 100$$

A sigmoidal dose-response curve is fitted to the % activities (% of control) vs compound concentration and IC50 values calculated using LabStats fit-curve in Excel.

### ACE ASSAY

Potency values for ACE or selectivity values for inhibitors of NEPi over ACE are determined by the following assay.

### THE PREPARATION AND ASSAY OF SOLUBLE ANGIOTENSIN CONVERTING ENZYME (ACE), FROM PORCINE AND HUMAN KIDNEY CORTEX

Soluble ACE activity is obtained from the kidney cortex and assayed by measuring the rate of cleavage of the ACE substrate Abz-Gly-p-nitro-Phe-Pro-OH to generate its fluorescent product, Abz-Gly.

1. MATERIALS

All water is double de ionised.

1.1 Human Kidney IIAM (Pennsylvania. U.S.A.) or UK Human Tissue Bank (UK HTB)

1.2 Porcine kidney ACE Sigma (A2580)

1.3 Homogenisation buffer-1

100 mM Mannitol and 20 mM Tris@pH 7.1

2.42 g Tris (Fisher T/P630/60) is diluted in 1 liter of water and the pH adjusted to 7.1 using 6M HCl at room temperature. To this 18.22 g Mannitol (Sigma M-9546) is added.

1.4 Homogenisation buffer-2

100 mM Mannitol, 20 mM Tris@pH7.1 and 10 mM MgCl₂.6H₂O (Fisher M0600/53)

To 500 ml of the homogenisation buffer 1 (1.4) 1.017 g of MgCl₂ is added.

1.5 Tris buffer (ACE buffer).

50 mM Tris and 300 mM NaCl@pH 7.4

50 ml of 50 mM Tris pH 7.4 (Sigma T2663) and 17.52 g NaCl (Fisher S/3160/60) are made up to 1000 ml in water.

1.6 Substrate (Abz-D-Gly-p-nitro-Phe-Pro-OH) (Bachem M-1100)

ACE substrate is stored as a powder at −20° C. A 2 mM stock is made by gently re-suspending the substrate in ACE buffer, this must not be vortexed or sonicated. 400 μl aliquots of the 2 mM stock are stored at −20° C. for up to one month.

1.7 Total product

Samples corresponding to 100% substrate to product conversion are included on the plate to enable the % substrate turnover to be determined (see calculations). The total product is generated by incubating 1 ml of 2 mM substrate with 20 μl of enzyme stock for 24 hours at 37° C.

1.8 Stop solution.

0.5M EDTA (Promega CAS[6081/92/6]) is diluted 1:250 in ACE buffer to make a 2 mM solution.

1.9 Dimethyl sulphoxide (DMSO).

1.10 Magnesium Chloride —MgCl₂.6H₂O (Fisher M0600/53).

1.11 Black 96 well flat bottom assay plates (Costar 3915 or Packard).

1.12 Topseal A (Packard 6005185).

1.13 Centrifuge tubes

2. SPECIFIC EQUIPTMENT

2.1 Sorvall RC-5B centrifuge (SS34 GSA rotor, pre-cooled to 4° C.).

2.2 Braun miniprimer mixer.

2.3 Beckman CS-6R centrifuge.

2.4 BMG Fluostar Galaxy.

2.5 Wesbart 1589 shaking incubator.

3. METHODS

3.1 TISSUE PREPARATION

3.3 Human ACE is obtained from the kidney cortex using a method adapted from Booth, A. G. & Kenny, A. J. (1974) Biochem. J. 142, 575-581.

3.3 Frozen kidneys are allowed to thaw at room temperature and the cortex is dissected away from the medulla.

3.4 The cortex is finely chopped and homogenised in approximately 10 volumes of homogenisation buffer-1 (1.4) using a Braun miniprimer (2.2).

3.5 Magnesium chloride (1.11) (20.3 mg/gm tissue) is added to the homogenate and stirred in an ice-water bath for 15 minutes.

3.6 The homogenate is centrifuged at 1,500 g (3,820 rpm) for 12 minutes in a Beckman centrifuge (2.3) before removing the supernatant to a fresh centrifuge tube and discarding the pellet.

3.7 The supernatant is centrifuged at 15,000 g (12,100 rpm) for 12 minutes in a Sovall centrifuge (2.1) and the supernatant is discarded.

3.8 The pale pink layer on the top of the remaining pellet is removed and re-suspended in homogenisation buffer-2 (1.5) (5 ml buffer per 1 g tissue).

3.9 The suspension is centrifuged at 2,200 g (4,630 rpm) for 12 minutes in a Beckman centrifuge before discarding the pellet.

3.10 The supernatant is centrifuged at 15,000 g (12,100 rpm) for 12 minutes using the Sorvall centrifuge and the supernatant is discarded.

3.11 The final pellet is resuspended in homogenisation buffer-2 (0.5 ml buffer per 1 g tissue). A homogenous suspension is obtained using a Braun miniprimer. This is then frozen down in 100 μl aliquots to be assayed for NEP activity.

4.0 DETERMINATION OF ACE ACTIVITY

The activity of the previously aliquoted ACE is measured by its ability to cleave the ACE specific peptide substrate.

Porcine ACE (1.2) is defrosted and resuspended in ACE buffer (1.6) at 0.004U/μl, this is frozen down in 50 μl aliquots.

4.1 A 4% DMSO/ACE buffer solution is made (4 mls DMSO in 96 mls ACE buffer).

4.2 Substrate (1.7), total product (1.8) and enzyme (1.1, 1.2, 1.3), are left on ice to thaw.

4.3 50 μl of 4% DMSO/ACE buffer solution is added to each well.

4.4 The 2 mM substrate stock is diluted 1:100 to make a 20 μM solution. 100 μl of 20 μM substrate is added to each well (final concentration in the assay 10 μM).

4.5 50 μl of a range of enzyme dilutions is added to initiate the reaction (usually 1:100, 1:200, 1:400, 1:800, 1:1600, and 1:3200 are used). 50 μl of ACE buffer is added to blank wells.

4.6 The 2 mM total product is diluted 1:200 to make 10 μM solution. 200 μl 10 μM product is added to the first four wells of a new plate.

4.7 Plates are incubated at 37° C. in a shaking incubator for 60 minutes.

4.8 The enzyme reaction is stopped by the addition of 100 μl 2 mM EDTA in ACE buffer and incubated at 37° C. in a shaking incubator for 20 minutes before being read on the BMG Fluostar Galaxy (ex320/em420).

5. ACE INHIBITION ASSAYS

5.1 Substrate, total product, and enzyme stocks are left on ice to thaw.

5.2 Compound stocks are made up in 100% DMSO and diluted 1:25 in ACE buffer to give a 4% DMSO solution. All further dilutions are carried out in a 4% DMSO/ACE buffer solution (4 mls DMSO in 96 mls ACE buffer).

5.3 50 μl of compound, in duplicate, is added to the 96 well plate and 50 μl of 4% DMSO/ACE buffer is added to control and blank wells.

5.4 Steps 5.2 and 5.3 can be carried out either by hand or using the Packard multiprobe robots

5.5 The 2 mM substrate stock is diluted 1:100 in ACE buffer to make a 20 μM solution (10 μM final concentration in the assay) (110 μl of 2 mM substrate added to 10.89 ml buffer is enough for 1 plate).

5.6 The enzyme stock is diluted in ACE buffer, as determined from activity checks (4.0).

5.7 The 2 mM total product stock is diluted 1:200 in ACE buffer to make a 10 μM solution. 200 μl is added to the first four wells of a separate plate.

5.8 The 0.5 mM EDTA stock is diluted 1:250 to make a 2 mM stock (44 μl EDTA to 10.96 ml ACE buffer).

5.9 To each well of the 96 well plate the following reagents are added:

TABLE 1
Reagents added to 96 well plate.

| | Compound/ | | Tris | | | ACE | Total |
|---|---|---|---|---|---|---|---|
| | DMSO | | Buffer | | Substrate | enzyme | product |
| Samples | 2 μl | compound | 50 | μl | 100 μl | 50 μl | None |
| Controls | 2 μl | DMSO | 50 | μl | 100 μl | 50 μl | None |
| Blanks | 2 μl | DMSO | 100 | μl | 100 μl | None | None |

| | | | | | | |
|---|---|---|---|---|---|---|
| Totals | 2 μl | DMSO | None | None | None | 200 μl |

5.10 50 μl of the highest concentration of each compound used in the assay is added in duplicate to the same 96 well plate as the totals (5.7). 150 μl of ACE buffer is added to determine any compound fluorescence.

5.11 The reaction is initiated by the addition of the ACE enzyme before incubating at 37° C. for 1 hour in a shaking incubator.

5.12 The reaction is stopped by the addition of 100 μl 2 mM EDTA and incubated at 37° C. for 20 minutes in a shaking incubator, before being read on the BMG Fluostar Galaxy (ex320/em420).

6. CALCULATIONS

The activity of the ACE enzyme is determined in the presence and absence of compound and expressed as a percentage.

FU=Fluorescence units

(i) % Control activity (turnover of enzyme):

$$\frac{Mean FU of controls - Mean FU of blanks}{Mean FU of totals - Mean FU of blanks} \times 100$$

(ii) % Activity with inhibitor:

$$\frac{Mean FU of compound - Mean FU of blanks}{Mean FU to totals - Mean FU of blanks} \times 100$$

(iii) Activity expressed as % of control:

$$\frac{\% Activity with inhibitor}{\% Control activity} \times 100$$

$$OR$$

$$\frac{Mean FU of compound - Mean FU of blanks}{Mean FU to totals - Mean FU of blanks} \times 100$$

(iv) % Inhibition=100−% control

(v) For fluorescent compounds the mean FU of blanks containing compound (5.10) is deducted from the mean FU of compound values used to calculate the % Activity.

A sigmoidal dose-response curve is fitted to the % activities (% of control) vs compound concentration and IC₅₀ values calculated using LabStats fit-curve in Excel. PDE action potency values referred to herein are determined by the following assays:

### PDE5 inhibitor—TEST METHODS

### Phosphodiesterase (PDE) Inhibitory Activity

Preferred PDE compounds suitable for use in accordance with the present invention are potent and selective cGMP PDE5 inhibitors. In vitro PDE inhibitory activities against cyclic guanosine 3′,5′-monophosphate (cGMP) and cyclic adenosine 3′,5′-monophosphate (cAMP) phosphodiesterases can be determined by measurement of their IC₅₀ values (the concentration of compound required for 50% inhibition of enzyme activity).

The required PDE enzymes can be isolated from a variety of sources, including human corpus cavernosum, human and rabbit platelets, human cardiac ventricle, human skeletal muscle and bovine retina, essentially by the method of W. J. Thompson and M. M. Appleman (Biochem., 1971, 10, 311). In particular, the cGMP-specific PDE (PDE5) and the cGMP-inhibited cAMP PDE (PDE3) can be obtained from human corpus cavernosum tissue, human platelets or rabbit platelets; the cGMP-stimulated PDE (PDE2) was obtained from human corpus cavernosum; the calcium/calmodulin (Ca/CAM)-dependent PDE (PDE1) from human cardiac ventricle; the cAMP-specific PDE (PDE4) from human skeletal muscle; and the photoreceptor PDE (PDE6) from bovine retina. Phosphodiesterases 7-11 can be generated from full length human recombinant clones transfected into SF9 cells.

Assays can be performed either using a modification of the “batch” method of W. J. Thompson et al. (Biochem., 1979, 18, 5228) or using a scintillation proximity assay for the direct detection of AMP/GMP using a modification of the protocol described by Amersham plc under product code TRKQ7090/7100. In summary, the effect of PDE inhibitors was investigated by assaying a fixed amount of enzyme in the presence of varying inhibitor concentrations and low substrate, (cGMP or cAMP in a 3:1 ratio unlabelled to [³H]-labeled at a conc ˜1/3 Kₘ) such that IC₅₀≅Kᵢ. The final assay volume was made up to 100 μl with assay buffer [20 mM Tris-HCl pH 7.4, 5 mM MgCl₂, 1 mg/ml bovine serum albumin]. Reactions were initiated with enzyme, incubated for 30-60 min at 30° C. to give <30% substrate turnover and terminated with 50 μl yttrium silicate SPA beads (containing 3 mM of the respective unlabelled cyclic nucleotide for PDEs 9 and 11). Plates were re-sealed and shaken for 20 min, after which the beads were allowed to settle for 30 min in the dark and then counted on a TopCount plate reader (Packard, Meriden, Conn.) Radioactivity units were converted to % activity of an uninhibited control (100%), plotted against inhibitor concentration and inhibitor IC₅₀ values obtained using the ‘Fit Curve’ Microsoft Excel extension.

### Functional activity

This can be assessed in vitro by determining the capacity of a compound of the invention to enhance sodium nitroprusside-induced relaxation of pre-contracted rabbit corpus cavernosum tissue strips, as described by S. A. Ballard et al. (Brit. J. Pharmacol., 1996, 118 (suppl.), abstract 153P).

### In Vivo Activity

Compounds were screened in anaesthetised dogs to determine their capacity, after i.v. administration, to enhance the pressure rises in the corpora cavernosa of the penis induced by intracavernosal injection of sodium nitroprusside, using a method based on that described by Trigo-Rocha et al. (Neurourol. and Urodyn., 1994, 13, 71).

NPY assay:

An assay for identifying NPY inhibitors is presented in WO-A-98/52890 (see page 96, lines 2 to 28).

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

By cross reference herein to compounds contained in patents which can be used in accordance with invention, we mean the therapeutically active compounds as defined in the claims (in particular of claim 1) and the specific examples (all of which is incorporated herein by reference).

1 The use of a neutral endopeptidase inhibitor (NEPi) compound in the preparation of a medicament for the treatment of male sexual dysfunction.

2. Use according to claim 1 for the treatment of ejaculatory disorders, desire disorders or male erectile dysfunction (MED).

3. Use according to claim 2 for the treatment of MED.

4. Use of a NEPi compound according to any one of claims 1 to 3 for the treatment of MED wherein the medicament is administered by mouth.

5. Use according to any one of the preceding claims wherein the NEPi has a selectivity for NEP over angiotensin converting enzyme (ACE) of greater than 100.

6. Use according to any of the preceding claims wherein the NEPi is a compound of formula I (or a pharmaceutically acceptable salt, solvate or prodrug thereof): 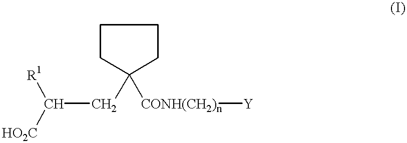
wherein
R¹ is C₁₋₆alkyl which may be substituted by one or more substituents, which may be the same or different, selected from the list: halo, hydroxy, C₁₋₆alkoxy, C₂₋₆ hydroxyalkoxy, C₁₋₆alkoxy(C₁₋₆alkoxy), C₃₋₇cycloalkyl, C₃₋₇cycloalkenyl, aryl, aryloxy, (C₁₋₄alkoxy)aryloxy, heterocyclyl, heterocyclyloxy, —NR²R³, —NR⁴COR⁵, —NR⁴SO₂R⁵, —CONR²R³, —S(O)ₚR⁶, —COR⁷ and −CO₂(C₁₋₄alkyl); or R¹ is C₃₋₇cycloalkyl, aryl or heterocyclyl, each of which may be substituted by one or more substituents from said list, which substituents may be the same or different, which list further includes C₁₋₆alkyl; or R¹ is C₁₋₆alkoxy, —NR²R³ or —NR⁴SO₂R⁵;
wherein
R² and R³ are each independently H, C₁₋₄alkyl, C₃₋₇cycloalkyl (optionally substituted by hydroxy or C₁₋₄alkoxy), aryl, (C₁₋₄alkyl)aryl, C₁₋₆alkoxyaryl or heterocyclyl; or R² and R³ together with the nitrogen to which they are attached form a pyrrolidinyl, piperidino, morpholino, piperazinyl or N-(C₁₋₄ alkyl)piperazinyl group;
R⁴ is H or C₁₋₄alkyl;
R⁵ is C₁₋₄alkyl, CF₃, aryl, (C₁₋₄alkyl)aryl, (C₁₋₄alkoxy)aryl, heterocyclyl, C₁₋₄alkoxy or —NR²R³ wherein R² and R³ are as previously defined;
R⁶ is C₁₋₄alkyl, aryl, heterocyclyl or NR²R³ wherein R² and R³ are as previously defined; and
R⁷ is C₁₋₄alkyl, C₃₋₇cycloalkyl, aryl or heterocyclyl; n is 0, 1 or 2; p is 0, 1, 2 or 3;
the —(CH₂)ₙ— linkage is optionally substituted by C₁₋₄alkyl, C₁₋₄alkyl substituted with one or more fluoro groups or phenyl, C₁₋₄alkoxy, hydroxy, hydroxy(C₁ ₃alkyl), C₃₋₇cycloalkyl, aryl or heterocyclyl;
Y is the group 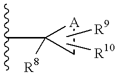
wherein A is —(CH₂)_{q}— where q is 1, 2, 3 or 4 to complete a 3 to 7 membered carbocyclic ring which may be saturated or unsaturated; R⁸ is H, C₁₋₆alkyl, —CH₂OH, phenyl, phenyl(C₁₋₄alkyl) or CONR¹¹R¹²; R⁹ and R¹⁰ are each independently H, —CH₂OH, —C(O)NR¹¹R¹², C₁₋₆alkyl, phenyl (optionally substituted by C₁₋₄alkyl, halo or C₁₋₄alkoxy or phenyl(C₁₋₄alkyl) wherein the phenyl group is optionally substituted by C₁₋₄alkyl, halo or C₁₋₄alkoxy, or R⁹ and R¹⁰ together form a dioxolane; R¹¹and R¹² which may be the same or different are H, C₁₋₄alkyl, R¹³ or S(O)ᵣR¹³, where r is 0, 1 or 2 and R¹³ is phenyl optionally substituted by C₁₋₄alkyl or phenylC₁₋₄alkyl wherein the phenyl is optionally substituted by C₁₋₄alkyl; or
Y is the group, —C(O) NR¹¹R¹² wherein R¹¹ and R¹² are as previously defined except that R¹¹ and R¹² are not both H; or
Y is the group, 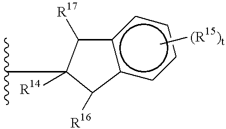
wherein R¹⁴ is H, CH₂OH, or C(O)NR¹¹R¹² wherein R¹¹ and R¹² are as previously defined; when present R¹⁵, which may be the same or different to any other R¹⁵, is OH, C₁₋₄alkyl, C₁₋₄alkoxy, halo or CF₃; t is 0, 1, 2, 3 or 4; and R¹⁶ and R¹⁷ are independently H or C₁₋₄alkyl; or
Y is the group 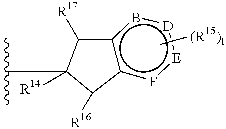
wherein one or two of B, D, E or F is a nitrogen, the others being carbon; and R¹⁴ to R¹⁷ and t are as previously defined; or
Y is an optionally substituted 5-7 membered heterocyclic ring, which may be saturated, unsaturated or aromatic and contains a nitrogen, oxygen or sulphur and optionally one, two or three further nitrogen atoms in the ring and which may be optionally benzofused and optionally substituted by:
C₁₋₆alkoxy; hydroxy; oxo; amino; mono or di-(C₁₋₄alkyl)amino;
C₁₋₄alkanoylamino; or
C₁₋₆alkyl which may be substituted by one or more substituents, which may be the same or different, selected from the list: C₁₋₆alkoxy, C₁₋₆haloalkoxy, C₁₋₆alkylthio, halogen, C₃₋₇cycloalkyl, heterocyclyl or phenyl; or
C₃₋₇cycloalkyl, aryl or heterocyclyl, each of which may be substituted by one or more substituents, which may be the same or different, selected from the list: C₁₋₆alkyl, C₁₋₆alkoxy, C₁₋₆haloalkoxy, C₁₋₆alkylthio, halogen, C₃₋₇cycloalkyl, heterocyclyl or phenyl;
wherein when there is an oxo substitution on the heterocyclic ring, the ring only contains one or two nitrogen atoms and the oxo substitution is adjacent a nitrogen atom in the ring; or Y is —NR¹⁸S(O)ᵤR¹⁹, wherein R¹⁸ is H or C₁₋₄alkyl; R¹⁹ is aryl, arylC₁₋₄alkyl or heterocyclyl (preferably pyridyl); and u is 0, 1, 2 or 3.

7. Use of a NEPi according to claim 6 wherein the NEPi is selected from the group consisting of:
2-[(1-{[(1-benzyl-6-oxo-1,6-dihydro-3-pyridinyl)amino]carbonyl}cyclopentyl)-methyl]-4-methoxybutanoic acid;
2-{[1-({[3-(2-oxo-1-pyrrolidinyl)propyl]amino}carbonylcyclopentyl]-methyl}-4-phenylbutanoic acid;
(+)-2-{[1-({[2-(hydroxymethyl)-2,3-dihydro-1H-inden-2-yl]amino}carbonyl)cyclopentyl]methyl}-4-phenylbutanoic acid;
2-[(1-{[(5-methyl-1,3,4-thiadiazol-2-yl)amino]carbonyl}cyclopentyl)methyl]-4-phenylbutanoic acid;
cis-3-(2-methoxyethoxy)-2-[(1-{[(4-{[(phenylsulfonyl)amino]carbonyl}cyclohexyl)-amino]carbonyl}cyclopentyl)methyl]propanoic acid;
(+)-2-{[1-({[2-(hydroxymethyl)-2,3-dihydro-1H-inden-2-yl]amino}carbonyl)cyclopentyl]-methyl}pentanoic acid;
(2R)-2-[(1-{[(5-ethyl-1,3,4-thiadiazol-2-yl)amino]carbonyl}cyclopentyl) methyl]pentanoic acid or (−)-2-[(1-{[(5-ethyl-1,3,4-thiadiazol-2-yl)amino]carbonyl}cyclopentyl)methyl]pentanoic acid;
(2S)-2-[(1-{[(5-ethyl-1,3,4-thiadiazol-2-yl)amino]carbonyl}cyclopentyl)-methyl]pentanoic acid or (+)-2-[(1-{[(5-ethyl-1,3,4-thiadiazol-2-yl)amino]carbonyl}cyclopentyl)-methyl]pentanoic acid; and
(S)-2-{[l -({[2-(hydroxymethyl)-2,3-dihydro-1H-inden-2-yl]amino}carbonyl)-cyclopentyl]methyl}-4-methoxybutanoic acid.

8. Use of pharmaceutical combination comprising a combination of a NEPi according to any preceding claim and;
one or more naturally occurring or synthetic prostaglandins or esters thereof; and/or
one or more α-adrenergic receptor antagonist compounds; and/or
one or more NO-donor (NO-agonist) compounds; and/or
one or more potassium channel openers or modulators; and/or
one or more dopaminergic agents; and/or
one or more vasodilator agents; and/or
one or more thromboxane A2 agonists; and/or
one or more ergot alkaloids; and/or
one or more compounds which modulate the action of natruretic factors in particular atrial naturetic factor; and/or
one or more angiotensin receptor antagonists; and/or
one or more substrates for NO-synthase; and/or
one or more calcium channel blockers; and/or
one or more antagonists of endothelin receptors and inhibitors or endothelin-converting enzyme; and/or
one or more cholesterol lowering agents such as statins and fibrates; and/or
one or more antiplatelet and antithrombotic agents; and/or
one or more insulin sensitising agents; and/or
one or more acetylcholinesterase inhibitors; and/or
one or more estrogen receptor modulators, estrogen agonists or estrogen antagonists; and/or
one or more of a PDE inhibitor, more particularly a PDE 2, 4, 5, 7 or 8 inhibitor; and/or
one or more of an NPY (neuropeptide Y) inhibitor, more particularly NPY1 or NPY5 inhibitor; and/or
one or more of vasoactive intestinal protein (VIP), VIP mimetic, VIP analogue; of a VIP receptor agonist or a VIP fragment, or a α-adrenoceptor antagonist with VIP combination; and/or
one or more of a melanocortin receptor agonist or modulator or melanocortin ehancer; and/or
one or more of a serotonin receptor agonist, antagonist or modulator; and/or
one or more of a testosterone replacement agent, dihydrotestosterone or a testosterone implant; and/or
one or more of estrogen, estrogen and medroxyprogesterone or medroxyprogesterone acetate (MPA) (i.e. as a combination); and/or
one or more of a modulator of transporters for noradrenaline, dopamine and/or serotonin; and/or
one or more of a purinergic receptor agonist and/or modulator; and/or
one or more of a neurokinin (NK) receptor antagonist; and/or
one or more of an opioid receptor agonist, antagonist or modulator, preferably agonists for the ORL-1 receptor; and/or
one or more of an agonist or modulator for oxytocin/vasopressin receptors,; and/or
one or more modulators of cannabinoid receptors.

9. Use according to claim 8 wherein the combination is that of a NEPi and a PDE5i for the treatment of male sexual dysfunction.

10. Use of a combination according to claim 9 which is for the treatment of MED.

11. Use of a combination according to claims 8 to 10 which is adapted for administration by mouth.

12. Use according to claims 9 to 11 wherein the PDE5i is selected from the group consisting of:
5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine;
5-(2-ethoxy-5-morpholinoacetylphenyl)-1-methyl-3-n-propyl-1,6-dihydro-7H-pyrazolo[4,3-d]pyrimidin-7-one;
3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one;
3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one;
(+)-3-ethyl-5-[5-(4-ethylpiperazin-1-ylsulphonyl)-2-(2-methoxy-1(R)-methylethoxy)pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1 R)-2-methoxy-1-methylethyl]oxy)pyridin-3-yl}-2-methyl-2,6-dihydro-7H-pyrazolo[4,3-d] pyrimidin-7-one;
5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine;
5-[2-iso-Butoxy-5-(4-ethylpiperazin-1-ylsu Iphonyl)pyridin-3-yl]-3-ethyl-2-(1-methylpiperidin-4-yl)-2,6-dihydro-7H-pyrazolo[4,3-d]pyrimidin-7-one;
5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one;
5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one;
5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one;
(6R, 12aR)-2,3,6,7,12,12a-hexahydro-2-methyl-6-(3,4-methylenedioxyphenyl) -pyrazino[2′,1′:6,1]pyrido[3,4-b]indole-1,4-dione (IC-351);
2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil) also known as 1-[[3-(3,4-dihydro-5-methyl-4-oxo-7-propylimidazo[5, 1-f]-as-triazin-2-yl)-4-ethoxyphenyl]sulphonyl]-4-ethylpiperazine; and
the compound of example 11 of published international application WO93/07124 (EISAI).

13. Use according to claim 12 wherein the PDE5i is sildenafil.

14. A pharmaceutical composition comprising a NEPi and a PDE5i for the treatment of MED.

15. A kit comprising a first component and a second component adapted for the treatment of MED wherein the first component comprises a NEPi as defined in any of claims 1 to 7 and wherein the second component comprises a PDE5i as defined in any of claims 9 to 13.

16. The use of a pharmaceutical combination adapted for administering by mouth in the preparation of a medicament for the treatment of male sexual dysfunction, said combination comprising an inhibitor of neutral endopeptidase (NEP) having an IC₅₀ against NEP of less than 100 nM and a selectivity for NEP over angiotensin converting enzyme of greater than 100, and an inhibitor of phosphodiesterase type 5 enzyme (PDE5) having an IC₅₀ against PDE5 of less than 100 nM and a selectivity for PDE5 over PDE3 of greater than 100.

17. A method for the treatment of male sexual dysfunction comprising administering to the patient an effective amount of a neutral endopeptidase inhibitor.

18. A method for the treatment of male sexual dysfunction comprising administering to the patient an effective amount of a neutral endopeptidase inhibitor and a phosphodiesterase type 5 inhibitor (PDE5).
